(19) United States
(12) Patent Application Publication
(10) Pub. No.: US 2011/0105544 A1
(43) Pub. Date: May 5, 2011

(54) CARBOXYLIC DERIVATIVES FOR USE IN THE TREATMENT OF CANCER

(76) Inventors: Juan Aymami Bofarull, Barcelona (ES); Franck Camille Nicolas Chevalier, Barcelona (ES); Montserrat Soler Lopez, Barcelona (ES); Maria Teresa Luque Garrofe, Barcelona (ES); Marc Martinell Pedemonte, Barcelona (ES)

(21) Appl. No.: 12/735,051

(22) PCT Filed: Dec. 18, 2008
(86) PCT No.: PCT/EP2008/067952

§ 371 Date: Jan. 3, 2011

(30) Foreign Application Priority Data
Dec. 21, 2007 (EP) 07150330.4

(51) Int. Cl. A61K 31/216; C07C 69/612; C07C 63/04; C07C 63/33; C07C 317/44; C07C 229/42; C07D 333/34; C07D 213/55; A61K 31/192; A61K 31/195; A61K 31/196; A61K 31/381; A61K 31/4402; A61P 35/00

(52) U.S. Cl. 514/277; 560/103; 562/493; 560/8; 562/405; 562/430; 560/12; 562/490; 562/457; 549/65; 546/342; 514/532; 514/570; 514/562; 514/533; 514/569; 514/563; 514/445

(57) ABSTRACT

The invention provides novel compounds of formula (I), wherein: R₁ is a radical derived from one of the known ring systems; R² is a phenyl radical optionally substituted; Xₙ represents a birradical selected from the group consisting of: —(CH₂)₁₋₄—, (C₂-C₄)-alkenyl, (C₂-C₄)alkynyl, —S—(CH₂)₁₋₃—#, and —(CH₂)₁₋₃—O—#; wherein the symbol # indicates the position at which Xₙ is attached to R₁; Yₙ is a birradical selected from the group consisting of: —(CH₂)₂₋₄—, —S—(CH₂)₁₋₃#, and —O—(CH₂)₁₋₃—#; wherein the symbol # indicates the position at which Yₙ is attached to R₂; and R³ is a radical selected from the group consisting of: —OR⁴. The compounds of formula (I) are useful in the treatment of cancer.

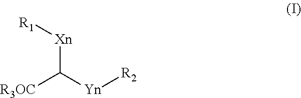

The invention relates to compounds of general formula (I), or pharmaceutically acceptable salts, which possess anti-proliferative activity and are therefore useful in methods of treatment of cancer.

### BACKGROUND ART

Cancer is a class of diseases or disorders characterized by uncontrolled division of cells and the ability of these cells to invade other tissues, either by direct growth into adjacent tissue through invasion or by implantation into distant sites by metastasis (in which cancer cells are transported through the blood or lymphatic system).

There are a series of types of cancer and the severity of symptoms depends on the site and character of the malignancy and the presence or absence of metastasis. Most cancers can be treated and some cured, depending on the specific type, location, and stage. The current therapies include surgery, chemotherapy, immunotherapy, hormone therapy, radiation therapy, and other treatment methods such as e.g. bone marrow transplantation, photodynamic therapy, and gene therapy.

The unregulated growth that characterizes cancer is caused primarily by damage to DNA, resulting in mutations of genes that encode proteins controlling cell division, apoptosis, and angiogenesis. Mutations are in general caused upon chemical or physical agents termed carcinogens, by close exposure to radioactive materials, or by certain viruses such as Human Papilloma Virus (HPV) or Epstein-Barr Virus (EBV), amongst others. Mutations occur spontaneously, or are hereditary and passed down generations as a result of germ line mutations.

Many forms of cancer are associated with exposure to environmental factors such as tobacco smoke, radiation, alcohol, and tumour-associated viruses. While some of these can be avoided, there is no known way to entirely avoid the disease.

Hence, cancer is still one of the leading causes of death in developed countries. In some Western countries, cancer is overtaking cardiovascular disease as the leading cause of death in spite of improved surgery and radiochemotherapy. In the last decades great efforts have been made to understand the molecular basis of cancer and to develop new therapies. As cancer has often a great impact on life quality of the patients and may lead to life threatening, there is still a need for developing new therapeutic agents with improved properties.

### SUMMARY OF THE INVENTION

The present inventors have found that the compounds of formula (I) has anti-proliferative activity. Owing to this anti-proliferative activity the compounds of the present invention are useful in the treatment of cancer.

Surprisingly, the inventors have found that the compounds of general formula (I) show a good anti-proliferative activity, being efficient in the treatment of cancer.

Thus, in a first aspect the present invention relates to a compound of general formula (I), or a pharmaceutical acceptable salt thereof, or a solvate thereof including a hydrate, or any stereoisomer or mixture of stereoisomers:

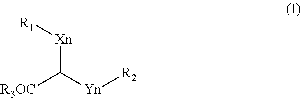

wherein:
R₁ is a radical derived from one of the known ring systems selected from the group consisting of:
one aromatic ring having 5-6 carbon atoms, being optionally one of said carbon atoms replaced by one N, O, or S atom; and
a two fused ring system, wherein
   one of the rings is aromatic and the other is aromatic or partially insaturated,
   each ring has 5-6 carbon atoms, being optionally 1-3 of said carbon atoms replaced by N, O, or S;
      wherein each ring, forming the known ring system, is optionally substituted by at least one radical selected from the group consisting of: (C₁-C₈)alkyl, —OH, halogen, (C₁-C₈)alkoxy, —CN, (C₁-C₈)fluoralkyl, (C₁-C₈)fluoroalkoxy, —CH₂—R₅, —R₁₀, -Qₙ-OR₅, -Qₙ-NR₄C(O)R₅, -Qₙ-C(O)NR₄R₆, -Qₙ-NH₂, -Qₙ-NR₄R₆, -Qₙ-S—R₆, -Qₙ-S(O₂)—R₆, -Qₙ-NR₄S(O₂)R₆, -Qₙ-S(O₂)NR₄R₆, -Qₙ-NR₄—CO, —NR₄R₅, -Qₙ-NR₄—CO—OR₅, and -Qₙ-O—CO—NR₄R₅;
where Qₙ is —(CH₂)ₙ—, being n=0, 1, 2 or 3;
   R₂ is a phenyl radical optionally substituted by at least one radical selected from the group consisting of: halogen, —OH, —Pₙ—OR₅, —NR₄C(O)R₆, —C(O)NR₄R₆, —NH₂, —NR₄R₅, —R₁₀, —R₆, —CN, (C₁-C₄)fluoralkyl, (C₁-C₄)fluoroalkoxy, (C₁-C₄)alkoxy, and (C₁-C₄)alkyl;
where Pₙ is —(CH₂)ₙ— being n=0 or 1
   Xₙ represents a birradical selected from the group consisting of: —(CH₂)₁₄—, (C₂-C₄)-alkenyl, (C₂-C₄)alkynyl, —S—(CH₂)₁₋₃—#, and —(CH₂)₁₋₃—O—#; wherein the symbol # indicates the position at which Xₙ is attached to R₁;
   Yₙ is a birradical selected from the group consisting of: —(CH₂)₂₋₄—, —S—(CH₂)₁₋₃#, and —O—(CH₂)₁₋₃—#; wherein the symbol # indicates the position at which Yₙ is attached to R₂;
   R₃ is a radical selected from the group consisting of: —OR₄, —O—CR₄R₄—O—C(O)—R₄;
   R₄ is a radical selected from: hydrogen and (C₁-C₄)alkyl optionally substituted by at least one radical selected from the group consisting of: —NH₂ and —OH;
   R₅ is a radical selected from: hydrogen, and -Lₙ-R₇, where
Lₙ is —(CH₂)ₙ— with n=0, 1, 2, 3 or 4; and
R₇ is a known ring systems with 1 ring or 2 fused rings, each one of the rings forming said ring system being partially unsaturated or aromatic, have 5-6 members, each member being independently selected from C, N, O, S, CH, CH₂, and NH; and being each ring forming said ring system optionally substituted by at least one radical selected from the group consisting of: (C₁-C₄)-alkyl, —CN, (C₁-C₄)fluoralkyl, (C₁-C₄)fluoroalkoxy, halogen, (C₁-C₄)alkoxy, —NH₂, —OH, dialkyl(C₁-C₄)amino, and a known aromatic ring of 5-6 members independently selected from N, O, S, CH, and NH which is linked to R₇ via a (C₁-C₄)alkyl birradical;
   R₆ is a radical selected from the group consisting of: (C₁-C₄)alkyl optionally substituted by at least one radical selected from the group consisting of: halogen, cyano, amino, and an aromatic known ring having 5-6 members independently selected from N, O, S, CH, and NH;
   R₁₀ is an aromatic known ring having 5-6 members independently selected from N, O, S, CH, and NH;
   with the proviso that when R₁ is phenyl:
   R₂ is a phenyl radical substituted by at least one radical selected from the group consisting of: (C₁-C₄)alkoxy, (C₁-C₄)alkyl, —NHC(O)CH₃, halogen, —O—CH₂—R₈, —OH, —NH₂, —OR₁₁, —R₈, —NHR₁₁, and —NH—CH₂-phenyl;
   R₈ is a aromatic known ring having 5-6 members independently selected from N, O, S, CH, and NH;
   R₁₁ is a phenyl ring optionally substituted with —F, —CF₃, —OCH₃ and —CN;
   R₃ is selected from the group consisting of: —OH, (C₁-C₄)alkoxy and —O—OH₂—O—C(O)—CH₃;
   Xₙ is selected from the group consisting of: —(OH₂)₃—; and
   Yₙ is selected from the group consisting of: —(CH₂)₂;
   and with the proviso that R₁ and R₂ are not simultaneously a phenyl substituted by one —OMe radical.

It is remarkable that the aromatic nature of both R₁ and R₂ enhances the activity of the compounds of the present invention as it is shown below. For this reason, it is important that R₁ has aromatic nature (in addition to R₂ which is phenyl optionally substituted), specially when R₁ is a 2-fused ring system: it is necessary that at least one of the rings forming the system is aromatic in order to achieve the therapeutic effect. In fact, the inventors of the present invention believe that from the activity data obtained with the compounds of the present invention other useful compounds could be obtained which differed from those provided in the present application in that R₂ is an aromatic ring having 5-6 carbon atoms (other than phenyl), being optionally one of said carbon atoms replaced by one N, O, or S atom; or a two fused ring system, wherein one of the rings is aromatic and the other is aromatic or partially insaturated, each ring has 5-6 carbon atoms, and being optionally 1-3 of said carbon atoms replaced by N, O, or S.

In a second aspect the present invention relates to a pharmaceutical composition comprising a therapeutically effective amount of the compound of formula (I) as defined in the first aspect of the invention, together with the appropriate amounts of pharmaceutical excipients or carriers.

In a third aspect the present invention relates to a compound of general formula (I) as defined in the first aspect of the invention, or a compound selected from the group consisting of:
2-Methyl-4-phenylbutanoic acid;
2-Ethyl-4-phenylbutanoic acid;
2-(2-Phenylethyl)pentanoic acid;
2-Benzyl-4-phenylbutanoic acid;
5-Phenyl-2-(2-phenylethyl)pentanoic acid;
Methyl 2-methyl-4-phenylbutanoate;
Methyl 2-ethyl-4-phenylbutanoate;
(Acetyloxy)methyl 2-benzyl-4-phenylbutanoate;
(Acetyloxy)methyl 5-phenyl-2-(2-phenylethyl)pentanoate; Sodium 2-(Benzylthio)-5-phenylpentanoate;
2-(Benzyloxy)-5-phenylpentanoic acid;
5-Phenyl-2-propylpentanoic acid;
(4E)-5-Phenyl-2-(2-phenylethyl)pent-4-enoic acid; and
6-Phenyl-2-(2-phenylethyl)hexanoic acid;
   for use as a medicament.

In a fourth aspect the present invention relates to a compound of general formula (I) as defined in the first aspect of the invention, or a compound selected from the group consisting of:
2-Methyl-4-phenylbutanoic acid;
2-Ethyl-4-phenylbutanoic acid;
2-(2-Phenylethyl)pentanoic acid;
2-Benzyl-4-phenylbutanoic acid;
5-Phenyl-2-(2-phenylethyl)pentanoic acid;
Methyl 2-methyl-4-phenylbutanoate;
Methyl 2-ethyl-4-phenylbutanoate;
(Acetyloxy)methyl 2-benzyl-4-phenylbutanoate;
(Acetyloxy)methyl 5-phenyl-2-(2-phenylethyl)pentanoate;
Sodium 2-(Benzylthio)-5-phenylpentanoate;
2-(Benzyloxy)-5-phenylpentanoic acid;
5-Phenyl-2-propylpentanoic acid;
(4E)-5-Phenyl-2-(2-phenylethyl)pent-4-enoic acid; and
6-Phenyl-2-(2-phenylethyl)hexanoic acid;
   for use in the treatment of cancer. This aspect can be also formulated as the use of a compound as defined in the fourth aspect of the invention for the manufacture of a medicament for the treatment of cancer.

In a further aspect, the present invention provides a compound as defined according to the first aspect of the invention for use as a medicine.

In a still further aspect, the present invention provides a compound as defined according to the first aspect of the invention, for use in the treatment of cancer. This aspect can be also formulated as the use of a compound as defined in the first aspect of the invention for the manufacture of a medicament for the treatment of cancer.

Another aspect of the present invention is to provide a method for the treatment of cancer which comprises administering to a mammal, preferably a human, a therapeutically effective amount of the compound, as defined in the first or fourth aspect of the invention, together with one or more pharmaceutically acceptable carriers, excipients, diluents or adjuvants.

Throughout the description and claims the word “comprise” and variations of the word, such as “comprising”, is not intended to exclude other technical features, additives, components, or steps. Additional objects, advantages and features of the invention will become apparent to those skilled in the art upon examination of the description or may be learned by practice of the invention. The following examples are provided by way of illustration, and are not intended to be limiting of the present invention.

### DETAILED DESCRIPTION OF THE INVENTION

In the present invention, the term “fluoralkyl” means a hydrocarbon chain wherein one or more hydrogen atoms are replaced by one or more fluorine atoms.

In the same way, the term “fluoroalkoxy” means an alcoxy wherein one or more hydrogen atoms are replaced by one or more fluorine atoms.

Illustrative non-limitative examples of known ring systems consisting of one ring are those derived from: cyclopropenyl, cyclobutenyl, cyclopentenyl, phenyl, aziridinyl, oxirenyl, thiiranyl, azetidinyl, oxetanyl, pyrrolyl, furanyl, and thiophenyl.

Illustrative non-limitative examples of known ring systems consisting of two rings totally fused, are those derived from benzofuran, isobenzofuran, indole, isoindole, indolizine, indoline, isoindoline, benzofurazan, benzothiofuran, heterocyclic chromene, isochromene, chroman, isochroman, quinoline, isoquinoline, and quinolizine.

As used herein, the symbols —C(O)—, —S(O)— and —S(O₂)— means, respectively:

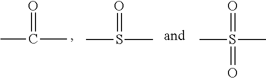

In one embodiment of the first aspect of the invention, the compound of formula (I) according to the first aspect of the invention is one wherein:

R₁ is a radical derived from one of the known ring systems selected from the group consisting of:
one aromatic ring having 5-6 carbon atoms, being optionally one of said carbon atoms replaced by one N, O or S; and
a two fused ring system, wherein
   one of the rings is aromatic and the other is aromatic or partially insaturated,
each ring has 5-6 carbon atoms, being optionally 1-3 carbon atoms replaced by N, O, or S;
   being each ring, forming said ring system, optionally substituted by at least one radical selected from the group consisting of: (C₁-C₈)alkyl, —OH, halogen, (C₁-C₈)alkoxy, —CN, —(C₁-C₄)fluoralkyl, —(C₁-C₄)fluoroalkoxy, —CH₂—R₅, —R₁₀, -Qₙ-OR₅, -Qₙ-NR₄C(O)R₅, -Qₙ-C(O)NR₄R₅, -Qₙ-NH₂, -Qₙ-NR₄R₅, -Qₙ-NR₄S(O₂)R₅, -Qₙ-S(O₂)NR₄R₅ and -Qₙ-NR₄—CO—OR₅;
   where Qₙ is —(CH₂)ₙ—, being n=0 or 1;
   R₄ is a radical selected from: hydrogen and (C₁-C₄)alkyl;
   R₅ is a radical selected from: hydrogen, and -Lₙ-R₇ where
Lₙ is —(CH₂)ₙ— with n=0 or 1; and
R₇ is an aromatic ring with 5-6 members, each member being independently selected from N, O, S, CH, and NH; and being optionally substituted by at least one radical selected from the group consisting of: —(C₁-C₄)-alkyl, —CN, —(C₁-C₄)fluoralkyl, —(C₁-C₄)fluoroalkoxy, halogen, —(C₁-C₄)alkoxy, —NH₂, —OH and dialkyl(Cᵣ C₄)amino.

In another embodiment of the first aspect of the invention, the compound of general formula (I) is one wherein:

R₁ is a radical derived from naphtyl; phenyl and thiophene,
being the phenyl and thiophene radical optionally substituted by at least one radical selected from the group consisting of: (C₁-C₄)alkyl, —OH, halogen, (C₁-C₄)alkoxy, —CN, —OF₃, —CHF₂, —OCF₃, —OCHF₂, —NH₂, —R₁₀, —NR₄C(O)R₅, —C(O)NR₄R₅, —NR₄S(O₂)R₅, and —S(O₂)NR₄R₅;
   R₂ is a phenyl radical optionally substituted by at least one radical selected from the group consisting of: halogen; —OH, —OR₅, —NHC(O)R₆, —C(O)NHR₆, —NH₂, —NHR₅, —R₁₀, —R₆, —CN, —CF₃, —CHF₂, —OCF₃, —OCHF₂, (C₁-C₄)alkoxy and (C₁-C₄)alkyl;
   Xₙ represents a birradical selected from the group consisting of: —(CH₂)₂₋₄—, (C₂-C₄)-alkenyl, (C₂-C₄)alkynyl, and —S—(CH₂)₁₋₃—#, wherein the symbol # indicates the position at which Xₙ is attached to R₁;
   Yₙ is a birradical selected from the group consisting of: —(CH₂)₂₋₃—, —S—(CH₂)₁₋₂#, and —O—(CH₂)₁₋₂-#; wherein the symbol # indicates the position at which Yₙ is attached to R₂;
   R₅ is a radical selected from: hydrogen and -Lₙ-R₇,
where Lₙ is —(CH₂)ₙ— being n=0 or 1; and
R₇ is an aromatic known ring system with 5-6 members, each member being independently selected from N, O, S, CH, and NH; and being optionally substituted by at least one radical selected from the group consisting of: (C₁-C₄)-alkyl, —CN, —CF₃, —CHF₂, —OCF₃, —OCHF₂, halogen, (C₁-C₄)alkoxy, —NH₂, —OH and dialkyl(C₁-C₄)amino.

In a more preferred embodiment of the first of the invention,

R₁ is a radical derived from naphtyl; phenyl and thiophene,
being the phenyl and thiophene radical optionally substituted by at least one radical selected from the group consisting of: —NR₄C(O)R₅, and —S(O₂)NR₄R₅;
   R₂ is a phenyl radical optionally substituted by at least one radical selected from the group consisting of: —OH, —OR₅, —NHR₅, —R₁₀, and (C₁-C₄)alkyl; and
   R₅ is hydrogen or a phenyl radical optionally substituted by at least one radical selected from the group consisting of: (C₁-C₄)-alkyl, —CN, (Oᵣ C₄)alkoxy, —CH₂-phenyl, —CH₂-pyridine, and dialkyl(C₁-C₄)amino.

In still another preferred embodiment of the first aspect of the invention,

R₁ is selected from the group consisting:

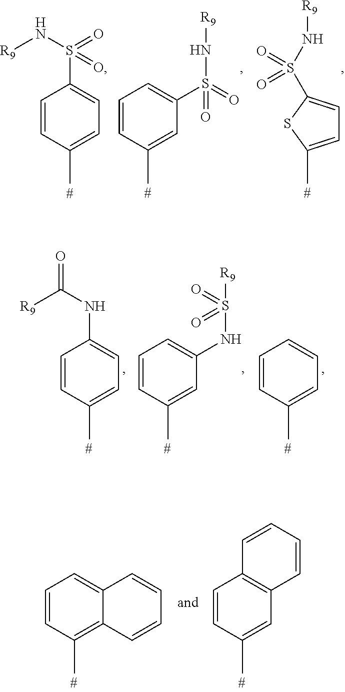

wherein the symbol # indicates the position at which R₁ is attached to Xₙ;
wherein R₉ is —CH₃, —Rₓ or —CH₂—Rₓ;

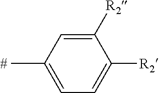

### R₂ is

wherein
the symbol # indicates the position at which R₂ is attached to Yₙ;
R″₂ is selected from the group consisting of: hydrogen, —OCH₃, —OH; and
R′₂ is selected from the group consisting of: hydrogen, —NH₂, (C₁-C₄)alkyl, —OH, —OCH₃, —CN, halogen, —NH—CO—CH₃, —O—R₅, —O—CH₂-phenyl, —O—CH₂-pyridine, —NH—CH₂-phenyl, and an aromatic known ring having 5-6 members selected from CH, N, NH, O and S;
R₅ is phenyl optionally substituted by one radical selected from: —CN, —F, —OCH₃, —CF₃;
   Rₓ is phenyl optionally substituted by one radical selected from the group consisting of: (C₁-C₄)alkyl, (C₁-C₄)alkoxy, and dialkyl(C₁-C₄)amino;
   Xₙ represents a birradical selected from the group consisting of: —(CH₂)₂₋₄—, C₃-alkenyl, —C₃-alkynyl, and —S—(CH₂)₂—#; wherein the symbol # indicates the position at which Xₙ is attached to R₁;
   Yₙ is a birradical selected from the group consisting of: —(CH₂)₂₋₃—, —SCH₂—#, and —OCH₂-#; wherein the symbol # indicates the position at which Xₙ is attached to R₂; and
   R₃ is hydroxyl, methoxy, ethoxy or —O—CH₂—O—C(O)—CH₃.

In another embodiment of the first aspect of the invention, the compound of general formula (I) is one where:

R₁ is selected from the group consisting of:

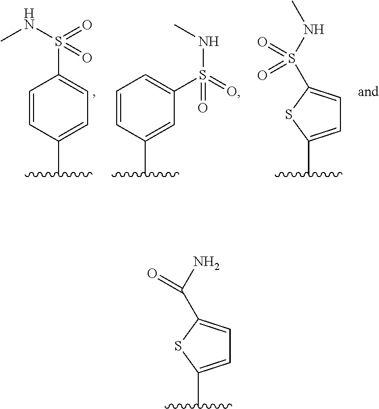

wherein the symbol [symbol] indicates the position at which R₁ is attached to Xₙ;
R₂ is selected from the group consisting of:

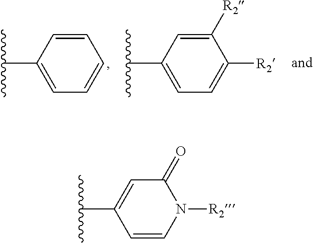

wherein R′₂ and R″₂ are selected from the group consisting of: hydrogen, —NH₂, —OH, —OCH₃, —Cl, —Br, —CONH₂, and phenyl; and the symbol [symbol] indicates the position at which R₂ is attached to Yₙ.
Xₙ is selected from the group consisting of: —(CH₂)—, —(CH₂)₂—, —(CH₂)₃—,

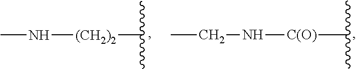

wherein the symbol [symbol] indicates the position at which Xₙ is attached to R₁;
Yₙ is selected from the group consisting of:

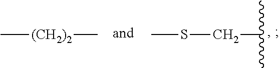

wherein the symbol [symbol] indicates the position at which Yₙ is attached to R₂; and
R₃ is hydroxyl, methoxy, ethoxy —O—CH₂—O—C(O)—CH₃, or

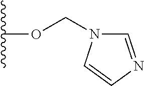

wherein the symbol [symbol] indicates the position wherein the group C═O is attached to R₃.

In another embodiment of the first aspect of the invention,

R₁ is phenyl;
R₂ is a phenyl radical substituted by at least one radical selected from the group consisting of: (C₁-C₄)alkoxy, (C₁-C₄)alkyl, —NHC(O)CH₃, halogen, —O—CH₂—R₈, —OH, —NH₂, —OR₁₁, —R₈, —NHR₁₁, and —NH—CH₂-phenyl;
R₈ is a aromatic known ring having 5-6 members independently selected from N, O, S, CH, and NH;
R₁₁ is a phenyl ring optionally substituted with —F, —CF₃, —OCH₃ and —CN;
R₃ is selected from the group consisting of: —OH, (C₁-C₄)alkoxy and —O—CH₂—O—C(O)—CH₃;
Xₙ is selected from the group consisting of: —(CH₂)₃—; and
Yₙ is selected from the group consisting of: —(CH₂)₂.

In another embodiment of the first aspect of the invention,

R₁ is phenyl substituted by at least one radical selected from —S(O₂)NR₄R₅, —NR₄S(O₂)R₅ and —NR₄C(O)R₅,
R₂ is phenyl optionally substituted by one (C₁-C₄)alkyl radical;
R₃ is selected from the group consisting of: —OH, methoxy, and —O—CH₂—O—C(O)—CH₃;
R₄ is hydrogen; and
R₅ is -Lₙ-R₇, where
Lₙ is —(CH₂)ₙ— being n=0 or 1; and
R₇ is phenyl optionally substituted by at least one radical selected from the group consisting of: (C₁-C₄)-alkyl, (C₁-C₄)alkoxy, and dialkyl(C₁-C₄)amino;

### Xₙ is

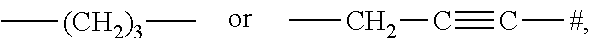

wherein the symbol # indicates the position at which Xₙ is attached to R₁; and

### Yₙ is —(CH₂)₂—.

In another embodiment of the first aspect of the invention,

R₁ is naphtyl;
R₂ is phenyl optionally substituted by one radical selected from the group consisting of: (C₁-C₄)alkyl radical, thiophene and pyridine;

### R₃ is —OH;

### Xₙ is —(CH₂)₃—; and

### Yₙ is —(CH₂)₂—.

In another embodiment of the first aspect of the invention,

R₁ is thiophene substituted by one —S(O₂)NR₄R₅ radical;
R₂ is phenyl;

### R₃ is —OH;

R₄ is hydrogen;
R₅ is a (C₁-C₄)alkyl radical;

### Xₙ is —(CH₂)₃—; and

### Yₙ is —(CH₂)₂—.

Preferably the compound of general formula (I) according to the first aspect of the invention is selected from the group consisting of:
2-{4-[(Methylamino)sulfonyl]benzyl}-4-phenylbutanoic acid;
5-(4-[(Methylamino)sulfonyl]phenyl)-2-(2-phenylethyl)pentanoic acid;
(Acetyloxy)methyl 5-(3-{[(3,4-dimethoxyphenyl)amino]sulfonyl}phenyl)-2-(2-phenylethyl)pentanoate;
(Acetyloxy)methyl 5-(3-[(4-methylanilinosulfonyl]phenyl)-2-(2-phenylethyl)pentanoate;
(Acetyloxy)methyl 5-(3-[(methylamino)sulfonyl]phenyl)-2-(2-phenylethyl)pentanoate;
Sodium 5-phenyl-2-(2-phenylethyl)pentanoate;
5-(2-Naphthyl)-2-(2-phenylethyl)pentanoic acid;
5-(1-Naphthyl)-2-(2-phenylethyl)pentanoic acid;
5-[3-{[4-(Dimethylamino)benzoyl]amino}phenyl]-2-(2-phenylethyl)pentanoic acid;
5-[3-{[4-(Dimethylamino)benzoyl]amino}phenyl]-2-(2-phenylethyl)pentanoic acid hydrochloride;
5-(3′-{[(4-Methylphenyl)sulfonyl]amino}phenyl)-2-(2-phenylethyl)pentanoic acid;
5-{5-[(Methylamino)sulfonyl]thien-2-yl}-2-(2-phenylethyl)pentanoic acid;
5-(3-[(Benzylamino)sulfonyl]phenyl)-2-(2-phenylethyl)pentanoic acid;
5-Phenyl-2-(2-pyridin-2-ylethyl)pentanoic acid;
2-[2-(3-methoxyphenyl)ethyl]-5-phenylpentanoic acid;
2-[2-(3-Hydroxyphenyl)ethyl]-5-phenylpentanoic acid;
2-{2-[4-(Acetylamino)phenyl]ethyl}-5-phenylpentanoic acid;
2-[2-(4-Aminophenyl)ethyl]-5-phenylpentanoic acid;
2-[2-(4-(Benzylamino)phenyl)ethyl]-5-phenylpentanoic acid;
5-(3-[(4-Methylanilinosulfonyl]phenyl)-2-(2-phenylethyl)pentanoic acid;
Sodium 5-(3-[(4-methylanilinosulfonyl]phenyl)-2-(2-phenylethyl)pentanoate;
5-[3-(4-Methylanilinosulfonyl)phenyl]-2-(2-phenylethyl)pent-4-ynoic acid;
5-(3-[(Anilinosulfonyl]phenyl)-2-(2-phenylethyl)pentanoic acid;
5-(4-[Anilinosulfonyl]phenyl)-2-(2-phenylethyl)pentanoic acid;
5-(3-[(Methylamino)sulfonyl]phenyl)-2-(2-phenylethyl)pentanoic acid;
Methyl 5-(3-[(methylamino)sulfonyl]phenyl)-2-(2-phenylethyl)pentanoate;
5-(3-{[(3,4-dimethoxyphenyl)amino]sulfonyl}phenyl)-2-(2-phenylethyl) pentanoic acid;
5-[3-(4-methylanilinosulfonyl)phenyl]-2-(3-phenylpropyl)pentanoic acid;
2-[2-(4-Ethylphenyl)ethyl]-5-phenylpentanoic acid;
5-[4-(Anilinosulfonyl)phenyl]-2-[2-(4-ethylphenyl)ethyl]pentanoic acid;
Methyl 5-[4-(anilinosulfonyl)phenyl]-2-[2-(4-ethylphenyl)ethyl]pentanoate;
2-[2-(4-Ethylphenyl)ethyl]-6-phenylhexanoic acid;
5-(3-{[4-(Dimethylamino)benzoyl]amino}phenyl)-2-[2-(4-ethylphenyl)ethyl]pentanoic acid;
5-(1-Naphthyl)-2-[2-(4-ethylphenyl)ethyl]-pentanoic acid;
2-[2-(4-hydroxyphenyl)ethyl]-5-phenylpentanoic acid;
2-[2-(4-Benzyloxyphenyl)ethyl]-5-phenylpentanoic acid;
(Acetyloxy)methyl 2-[2-(4-benzyloxyphenyl)ethyl]-5-phenylpentanoate;
2-[2-(4-Methoxyphenyl)ethyl]-5-phenylpentanoic acid;
2-{2-[4-(Pyridin-2-ylmethoxy)phenyl]ethyl}-5-phenylpentanoic acid;
2-{2-[4-(Pyridin-4-ylmethoxy)phenyl]ethyl}-5-phenylpentanoic acid;
2-{2-[4-(2-Cyanophenoxy)phenyl]ethyl}-5-phenylpentanoic acid;
2-{2-[4-(3-cyanophenoxy)phenyl]ethyl}-5-phenylpentanoic acid;
2-{2-[4-(4-cyanophenoxy)phenyl]ethyl}-5-phenylpentanoic acid;
2-{2-[4′-(2-Furyl)phenyl]ethyl}-5-phenylpentanoic acid;
2-{2-[4′-(3-Furyl)phenyl]ethyl}-5-phenylpentanoic acid;
2-{2-[4′-(3-Thienyl)phenyl]ethyl}-5-phenylpentanoic acid;
2-{2-[(4′-Pyridinyl)phenyl-4-yl]ethyl}-5-phenylpentanoic acid;
2-{2-[(4′-Pyridinyl)phenyl-4-yl]ethyl}-5-phenylpentanoic acid hydrochloride;
2-[2-(4-Pyridin-3′-ylphenyl)ethyl]-5-phenylpentanoic acid;
5-Phenyl-2-[2-(4′-thien-2-ylphenyl)ethyl]pentanoic acid;
2-[2-(1,1′-Biphenyl-4-yl)ethyl]-5-phenylpentanoic acid;
5-(1-Naphthyl)-2-[2-(4′-thien-2-ylphenyl)ethyl]-pentanoic acid;
5-(1-Naphthyl)-2-[2-(4′-pyridin-3-ylphenyl)ethyl]-pentanoic acid;
2-{2-[4-(Pyridin-3-ylmethoxy)phenyl]ethyl}-5-phenylpentanoic acid;
2-{2-[4-(4-Fluorophenoxy)phenyl]ethyl}-5-phenylpentanoic acid;
2-{2-[4-Phenoxyphenyl]ethyl}-5-phenylpentanoic acid;
2-{2-[4-(4-Trifluoromethylphenoxy)phenyl]ethyl}-5-phenylpentanoic acid;
2-[2-(4-Bromophenyl)ethyl]-5-phenylpentanoic acid;
2-{2-[4-(4-Methoxyphenoxy)phenyl]ethyl}-5-phenylpentanoic acid;
2-[2-(2,3-Dihydro-1H-indol-3-yl]ethyl]-5-phenylpentanoic acid;
5-(1-Naphthyl)-2-[2-(4′-pyridin-3-ylphenyl)ethyl]-pentanoic acid; and
2-[2-(4-Anilinophenyl)ethyl]-5-phenylpentanoic acid.

The compounds of the present invention are obtained carrying out the steps summarized in the following scheme 1:

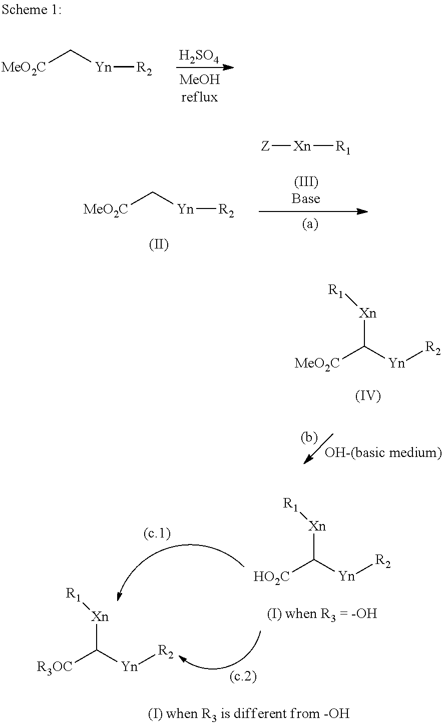

Step (a) corresponds to the alkylation of a compound of general formula (II) with a compound of general formula (III), wherein Z represents halogen. This reaction is carried out in the presence of a suitable base, for instance LDA, although other bases can be used as it is well-known for those skilled in the art.

When it is used as electrophile of formula (III) an alkenyl or alkenyl halide, the resulting product of the previous step can be subjected to hydrogenation.

Step (b) corresponds to the hydrolysis of the product resulting from the alkylation of step (a) or from the hydrogenation. This step is carried out in a basic medium, such as an alkaline or alkaline earth metal hydroxide.

When the compound of general formula (I) is one wherein R₃ is different from —OH (for instance an alkoxy or an amine), an additional step, (c), is carried out. This step consists of:
c.1) Alkylation in presence of base (such as triethylamine); or
c.2) Esterification in the presence of coupling reagents.

Suitable bases and coupling agents are well-known in the state of the art.

When the compound of formula (I) is one wherein R₂ is phenyl substituted by R₁₀ (i.e. a known ring system as defined above), it can be obtained following the routes summarizes in the following Scheme 2:

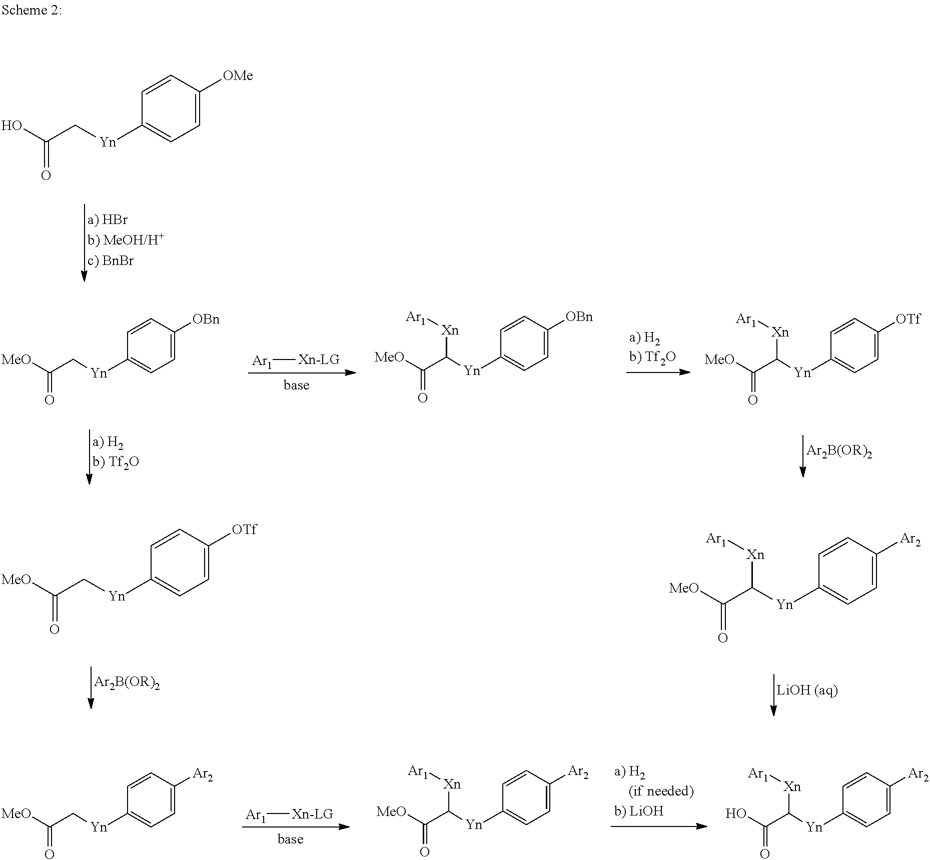

wherein:
Ar₁=corresponds to R1;
Ar₂=is an aromatic known ring having 5-6 members independently selected from N, O, S, CH, and NH
Ar₂B(OR)₂=boronic acid based reagent for the introduction of Ar₂; and
LG=is a leaving group such as halogen
and being Xₙ and Yₙ as defined above.

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal by implant of a depot, for example, subcutaneously or intramuscularly. The active compound of the present invention can be administered in the form of an acid or in the form of a pharmaceutically acceptable prodrug. “A pharmaceutically acceptable prodrug” is a compound that may be converted under physiological conditions or by solvolysis to the specified compound or to a pharmaceutically acceptable salt of such compound.

The compounds according to the present invention have the ability of binding to the histone deacetylase (HDAC), inhibiting its activity. Histone deacetylases are a family of enzymes that can deacetylate acetyl lysines. HDACs are primarily involved in regulation of chromatin structure and gene expression via their ability to modulate histone acetylation, although they also play roles in other important cellular functions like microtubule deacetylation. As it is well-known in the state of the art, HDAC inhibitors have shown activity in numerous disease models including, but not limited to, many cancers such as colon cancer, lymphoma, leukemias, lung cancer, breast cancer, prostate cancer; neurogenerative disease like Spinal Muscular Atrophy, Huntington Disease, Alzheimer's disease, immune disorder including graft vs. host disorders, Multiple Sclerosis, renal disease, Nervous system conditions, diabetes, malaria, HIV, and stimulation of stem cells/reprogramming of somatic cells to induce pluripotent stem cells. Therefore, the compounds of the present invention not only are useful for the treatment of cancer but also for the treatment of a pathology associated with an increase of HDAC activity and/or HDAC protein levels.

In one embodiment of the present invention, it is provided a compound of formula (I) as defined in the first aspect of the invention for the treatment of neurodegenerative diseases. This aspect can be formulated as the use of a compound of formula (I) for the manufacture of a medicament for the treatment of a neurodegenerative disease. Preferably, the neurodegenerative disease is selected from Alzheimer's disease, Parkinson's disease, Huntington disease, Lewy Body dementia, and Spinal Muscular Atrophy.

The invention further provides a method for the treatment of a neurodegenerative disease which comprises administering to a mammal, preferably a human, a therapeutically effective amount of the compound, as defined in the first or fourth aspect of the invention, together with one or more pharmaceutically acceptable carriers, excipients, diluents or adjuvants.

The pharmaceutical composition (e.g. formulation) may comprise a therapeutically effective amount of the compound of formula (I), as defined above, together with one or more pharmaceutically acceptable excipients or carriers such as adjuvants, diluents, fillers, buffers, stabilizers, preservatives, lubricants.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The term “therapeutically-effective amount,” as used herein, pertains to that amount of an active compound, or a material, composition or dosage form comprising an active compound, which is effective for producing some desired therapeutic effect.

In one embodiment of the third aspect of the invention, the compounds can be used as an anti-cancer agent.

The term “anti-cancer agent” as used herein, pertains to a compound which treats a cancer (i.e., a compound which is useful in the treatment of a cancer). The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a turnout from its origin), the inhibition of invasion (the spread of turnout cells into neighboring normal structures), or the promotion of apoptosis (programmed cell death).

In one embodiment of the fourth aspect of the invention, the cancer is selected from breast cancer and colon cancer.

The subject may be a eukaryote, an animal, a vertebrate animal, a mammal, a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutang, gibbon), or a human.

### EXAMPLES

Molecule names were generated using IsisDraw version 2.4. For molecules larger than 50 atoms, the molecule was fragmented to generate the name. In the case of a conflict between a name and a drawing of the structure, the drawing is controlling.

### Synthesis of Intermediates

R₁=Phenyl. Methyl 2-{2-(4-{[(trifluoromethyl)sulfonyl]oxy}phenyl)ethyl}-5-phenylpentanoate (Intermediate A).

### a) 4-(4-Hydroxyphenyl)butanoic acid

A suspension of 4-(4-methoxyphenyl)butanoic acid (2.0 g, 9.603 mmol) in HBr (20 mL, 48% aqueous solution) was refluxed for 2 h. The reaction mixture was allowed to reach room temperature (hereinafter abbreviated as “r.t.”), poured into H₂O (150 mL) and extracted with EtOAc (200 mL). The organic layer was dried over Na₂SO₄ (anhydrous), filtered and concentrated, to give 1.81 g of 4-(4-hydroxyphenyl)butanoic acid (white solid). The crude residue was submitted to next step without purification.

### b) Methyl 4-(4-hydroxyphenyl)butanoate

H₂SO₄ (2 mL, 37.32 mmol) was added to a solution of 4-(4-hydroxyphenyl)butanoic acid (9.603 mmol) in MeOH (40 mL). The reaction mixture was refluxed for 1 h, allowed to reach r.t., and poured into H₂O (150 mL). It was extracted with CH₂Cl₂ (200 mL). The organic layer was dried over Na₂SO₄ (anhydrous), filtered and concentrated, to give 1.59 g of crude methyl 4-(4-hydroxyphenyl)butanoate (colourless oil, yield: 85%). The compound was submitted to next step without further purification.

¹H NMR (CDCl₃, 250 MHz) δ ppm: 7.02 (d, J=7.9 Hz, 2H), 6.75 (d, J=7.9 Hz, 2H), 5.47 (bs, 1H), 3.67 (s, 3H), 2.57 (t, J=7.7 Hz, 2H), 2.32 (t, J=7.7 Hz, 2H), 1.92 (m, 2H).

### c) Methyl 4-[4-(benzyloxy)phenyl]butanoate

BnBr (2.5 mL, 21.018 mmol) was added to a suspension of K₂CO₃ (3.0 g, 21.706 mmol) and methyl 4-(4-hydroxyphenyl)butanoate (2.10 g, 10.812 mmol) in CH₃CN (100 mL). The reaction mixture was stirred at r.t. overnight (18 h). It was poured into H₂O (200 mL) and extracted with EtOAc (150 mL). The organic layer was dried over Na₂SO₄ (anhydrous), filtered and concentrated. The crude residue was flash chromatographed on SiO₂ (0→5% EtOAc/hexanes), to afford 3.05 g of methyl 4-[4-(benzyloxy)phenyl]butanoate (colourless oil, yield: 99%).

¹H NMR (CDCl₃, 250 MHz) δ ppm: 7.38 (m, 5H), 7.10 (d, J=8.5 Hz, 2H), 6.90 (d, J=8.5 Hz, 2H), 5.05 (s, 2H), 3.66 (s, 3H), 2.59 (m, 2H), 2.31 (m, 2H), 1.92 (m, 2H).

### d) Methyl (4E)-2-[2-(4-benzyloxyphenyl)ethyl]-5-phenylpent-4-enoate

A solution of methyl 4-[4-(benzyloxy)phenyl]butanoate (3.0 g, 10.550 mmol) in THF (5 mL) was added to a −78° C. cooled solution of LDA (13 mL, 1 M THF solution, 13 mmol) in THF (30 mL). The reaction mixture was stirred at low temperature for 2 min, and a solution of [(1E)-3-bromoprop-1-enyl]benzene (3.30 g, 16.744 mmol) in THF (5 mL) was added. The reaction was allowed to reach r.t. overnight (18 h). It was poured into H₂O (150 mL), taken up to pH=2 with HCl and extracted with EtOAc (150 mL). The organic layer was dried over Na₂SO₄ (anhydrous), filtered and concentrated. The crude residue was flash chromatographed on SiO₂ (2→4% EtOAc/hexanes), to give 3.51 g of methyl (4E)-2-[2-(4-benzyloxyphenyl)ethyl]-5-phenylpent-4-enoate (colourless oil, yield: 83%).

¹H NMR (CDCl₃, 250 MHz) δ ppm: 7.43-7.11 (m, 11H), 7.04 (d, J=8.5 Hz, 2H), 6.83 (d, J=8.5 Hz, 2H), 4.99 (s, 2H), 3.63 (s, 3H), 2.56-2.23 (m, 5H), 1.89 (m, 1H), 1.75 (m, 1H).

### e) Methyl 2-[2-(4-hydroxyphenyl)ethyl]-5-phenylpentanoate

Methyl (4E)-2-[2-(4-benzyloxyphenyl)ethyl]-5-phenylpent-4-enoate (3.45 g, 8.613 mmol) was added to a suspension of Pd/C (900 mg, 10% Pd/C, 0.845 mmol) in MeOH (60 mL). The reaction mixture was stirred under H₂ atmosphere (balloon) for 8 h. It was filtered through Celite (eluted with EtOAc) and solvent was concentrated off. The crude residue was flash chromatographed on SiO₂ (20% EtOAc/hexanes), to furnish 2.34 g of methyl 2-[2-(4-hydroxyphenyl)ethyl]-5-phenylpentanoate (white solid, yield: 87%).

¹H NMR (CDCl₃, 250 MHz) δ ppm: 7.28-7.07 (m, 5H), 6.96 (d, J=8.5 Hz, 2H), 6.70 (d, J=8.5 Hz, 2H), 5.00 (bs, 2H), 3.64 (s, 3H), 2.59-2.32 (m, 5H), 1.88 (m, 1H), 1.75-1.43 (m, 5H).

### f) Methyl 2-{2-(4-{[(trifluoromethyl)sulfonyl]oxy}phenyl)ethyl}-5-phenylpentanoate (Intermediate A)

Trifluoromethanesulfonic anhydride (2.60 g, 9.21 mmol) was added to a −18° C. cooled solution of methyl 2-[2-(4-hydroxyphenyl)ethyl]-5-phenylpentanoate (2.30 g, 7.361 mmol) and DIPEA (2.6 mL, 15.187 mmol) in CH₂Cl₂ (45 mL). The reaction mixture was allowed to react at low temperature for 10 min, poured into H₂O (150 mL), taken up to pH=3 with HCl and extracted with CH₂Cl₂ (120 mL).

The crude residue was purified by flash chromatography on SiO₂ (2 6% EtOAc/hexanes), to give 3.04 g of methyl 2-{2-(4-{[(trifluoromethyl)sulfonyl]oxy}phenyl)ethyl}-5-phenylpentanoate (colourless oil, yield: 93%).

¹H NMR (CDCl₃, 250 MHz) δ ppm: 7.37-7.17 (m, 9H), 3.73 (s, 3H), 2.63 (m, 4H), 2.45 (m, 1H), 1.99 (m, 1H), 1.86-1.48 (m, 5H).

R₁=Naphthyl. Methyl 2-[2-(4-{[(trifluoromethyl)sulfonyl]oxy}phenyl)ethyl]-5-(1-naphthyl)pentanoate (Intermediate B).

### a) Methyl 2-[2-(4-benzyloxyphenyl)ethyl]-5-(1-naphthyl)pent-4-ynoate

A solution of methyl 4-[4-(benzyloxy)phenyl]butanoate (1.5 g, 5.27 mmol) in THF (5 mL) was added to a −78° C. cooled solution of LDA (6 mL, 1 M THF solution, 6 mmol) in THF (30 mL). The reaction mixture was stirred at low temperature for 3 min, and a solution of 1-(3-bromoprop-1-ynyl)naphthalene (1.68 g, 6.86 mmol) in THF (5 mL) was added. The reaction was allowed to reach r.t. and stirred for 6 h. It was poured into H₂O (100 mL), taken up to pH=2 with HCl and extracted with EtOAc (2×100 mL). The organic layer was dried over Na₂SO₄ (anhydrous), filtered and concentrated. The crude residue was flash chromatographed on SiO₂ (5→10% EtOAc/hexanes), to furnish 950 mg of methyl 2-[2-(4-benzyloxyphenyl)ethyl]-5-(1-naphthyl)pent-4-ynoate (colourless oil, yield: 40%).

¹H NMR (CDCl₃, 250 MHz) δ ppm: 8.29 (m, 1H), 7.80 (m, 3H), 7.62-7.48 (m, 3H), 7.47-7.32 (m, 5H), 7.14 (d, J=8.8 Hz, 2H), 6.91 (d, J=8.8 Hz, 2H), 5.04 (s, 2H), 3.75 (s, 3H), 2.82 (m, 3H), 2.67 (m, 2H), 2.13 (m, 2H).

### b) Methyl 2-[2-(4-hydroxyphenyl)ethyl]-5-(1-naphthyl)pentanoate

Methyl 2-[2-(4-benzyloxyphenyl)ethyl]-5-(1-naphthyl)pent-4-ynoate (940 mg, 2.095 mmol) was added to a suspension of Pd/C (220 mg, 10% Pd/C, 0.210 mmol) in MeOH (20 mL). The reaction mixture was stirred under H₂ atmosphere (balloon) for 2 h. It was filtered through Celite (eluted with EtOAc) and solvent was concentrated off. The crude residue was flash chromatographed on SiO₂ (20→30% EtOAc/hexanes), to furnish 579 mg of methyl 2-[2-(4-hydroxyphenyl)ethyl]-5-(1-naphthyl)pentanoate (colourless oil, yield: 76%).

¹H NMR (CDCl₃, 250 MHz) δ ppm: 7.99 (m, 1H), 7.84 (m, 1H), 7.71 (d, J=7.7 Hz, 1H), 7.48 (m, 2H), 7.39 (m, 1H), 7.27 (m, 1H), 7.01 (d, J=7.4 Hz, 2H), 6.74 (d, J=7.4 Hz, 2H), 5.10 (bs, 1H), 3.67 (s, 3H), 3.04 (m, 2H), 2.49 (m, 3H), 1.93 (m, 1H), 1.73 (m, 5H).

### c) Methyl 2-[2-(4-{[(trifluoromethyl)sulfonyl]oxy}phenyl)ethyl]-5-(1-naphthyl)pentanoate (Intermediate B)

Trifluoromethanesulfonic anhydride (530 mg, 1.89 mmol) was added to a −18° C. cooled solution of methyl 2-[2-(4-hydroxyphenyl)ethyl]-5-(1-naphthyl) pentanoate (570 mg, 1.57 mmol) and DIPEA (430 mg, 3.37 mmol) in CH₂Cl₂ (30 mL). The reaction mixture was allowed to react at low temperature for 15 min, poured into H₂O (150 mL), taken up to pH=3 with HCl and extracted with CH₂Cl₂ (100 mL). The organic layer was dried over Na₂SO₄ (anhydrous), filtered and concentrated. The crude residue was purified by flash chromatography on SiO₂ (2→10% EtOAc/hexanes), to give 645 mg of methyl 2-[2-(4-{[(trifluoromethyl)sulfonyl]oxy}phenyl)ethyl]-5-(1-naphthyl)pentanoate (colourless oil, yield: 83%).

¹H NMR (CDCl₃, 250 MHz) δ ppm: 7.99 (m, 1H), 7.85 (m, 1H), 7.71 (d, J=7.7 Hz, 1H), 7.48 (m, 2H), 7.38 (t, J=7.8 Hz, 1H), 7.28 (m, 1H), 7.18 (m, 4H), 3.66 (s, 3H), 3.05 (m, 2H), 2.59 (m, 2H), 2.44 (m, 1H), 1.96 (m, 1H), 1.74 (m, 5H).

### Example 1

### Preparation of methyl 4-phenylbutanoate

H₂SO₄ (7.16 ml, 73.08 mmol) was added to a solution of 4-phenylbutanoic acid (40.0 g, 243.60 mmol) in MeOH (300 ml). The reaction mixture was stirred at room temperature for 1 h, poured into H₂O (500 ml) and extracted with CH₂Cl₂ (2×400 ml). The organic layer was washed with NaHCO₃ (300 ml, saturated aqueous solution), dried over Na₂SO₄ (anhydrous), filtered and concentrated. The crude residue was flash chromatographed on SiO₂ (10→20% EtOAc/hexanes), to furnish methyl 4-phenylbutanoate (41.95 g, colourless oil, yield: 96%).

¹H NMR (CDCl₃, 250 MHz) δ ppm: 7.28 (m, 2H), 7.20 (m, 3H), 3.68 (s, 3H), 2.67 (t, J=7.4 Hz, 2H), 2.35 (t, J=7.4 Hz, 2H), 1.99 (m, 2H).

### Example 2

### methyl 2-methyl-4-phenylbutanoate (Method A: ester alkylation)

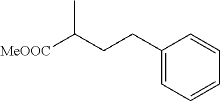

A solution of methyl 4-phenylbutanoate (500 mg, 2.80 mmol) in THF (5 ml) was dropwise added to a −78° C. cooled solution of freshly prepared LDA (1 M in THF, 3.08 ml, 3.08 mmol) in THF (10 ml). The reaction mixture was stirred at low temperature for 10 min, and MeI (0.23 ml, 3.64 mmol) was added. The reaction was allowed to stir at low temperature until full conversion was achieved (30 min, checked by TLC analysis).

The mixture was poured into H₂O (50 ml) and extracted with EtOAc (100 ml). The organic layer was dried over Na₂SO₄ (anhydrous), filtered and concentrated. The crude residue was flash chromatographed on SiO₂ (10% EtOAc/hexanes), to afford methyl 2-methyl-4-phenylbutanoate (440 mg, colourless oil, yield: 82%).

¹H NMR (CDCl₃, 250 MHz) δ ppm: 7.28 (m, 2H), 7.18 (m, 3H), 3.69 (2, 3H), 2.61 (t, J=8.2 Hz, 2H), 2.49 (m, 1H), 2.10-1.94 (m, 2H), 1.81-1.62 (m, 2H), 1.20 (d, J=7.1 Hz, 3H).

### Example 3

### 2-Methyl-4-phenylbutanoic acid (Method B: ester hydrolysis)

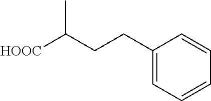

LiOH (5.72 ml, 2M solution in H₂O, 11.44 mmol) was added to a solution of methyl-4-phenylbutanoate (440 mg, 2.28 mmol) in a mixture of THF (10 ml) and MeOH (10 ml). The reaction mixture was warmed up to reflux, and stirred until no unreacted ester was detected by TLC analysis (30 min). The reaction mixture was allowed to reach room temperature and poured into H₂O (100 ml). It was acidified with HCl (10% aqueous solution) until pH 2-3, and extracted with EtOAc (100 ml). The organic layer was dried over Na₂SO₄ (anhydrous), filtered and concentrated. The crude residue was purified by flash chromatography on SiO₂ (10° 50% EtOAc/hexanes) to furnish 2-methyl-4-phenylbutanoic acid (225 mg, colourless oil, yield: 55%).

¹H NMR (CDCl₃, 250 MHz) δ ppm: 7.29 (m, 2H), 7.21 (m, 3H), 2.68 (t, J=8.2 Hz, 2H), 2.52 (m, 1H), 2.15-1.98 (m, 1H), 1.83-1.68 (m, 1H), 1.26 (d, J=6.8 Hz, 3H).

### Example 4

### Methyl 2-ethyl-4-phenylbutanoate

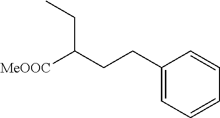

This compound was prepared following the same procedure than the one followed in Example 2, using EH as alkylating reagent instead of MeI. Flash chromatography purification afforded the title compound as a colourless oil (yield: 49%).

¹H NMR (CDCl₃, 250 MHz) δ ppm: 7.23 (m, 5H), 3.69 (2, 3H), 2.60 (m, 2H), 2.32 (m, 1H), 1.92 (m, 1H), 1.84-1.47 (m, 4H), 0.89 (t, J=7.4 Hz, 3H).

### Example 5

### 2-Ethyl-4-phenylbutanoic acid

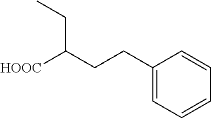

This compound was prepared following the same procedure than the one followed in Example 3, using as starting ester the methyl 2-ethyl-4-phenylbutanoate, to furnish the title compound as a colourless oil after flash chromatography purification (yield: 37%).

¹H NMR (CDCl₃, 250 MHz) δ ppm: 7.19 (m, 2H), 7.11 (m, 3H), 2.68-2.48 (m, 2H), 2.30 (m, 1H), 1.90 (m, 1H), 1.79-1.45 (m, 2H), 1.19 (m, 1H), 0.89 (t, J=7.4 Hz, 3H).

### Example 6

### Methyl 2-(2-phenylethyl)pentanoate

This compound was prepared following the same procedure than the one followed in Example 2, using PrI as alkylating reagent instead of MeI. Flash chromatography purification afforded the title compound as a colourless oil (yield: 45%).

¹H NMR (CDCl₃, 250 MHz) δ ppm: 7.35 (m, 2H), 7.25 (m, 3H), 3.76 (s, 3H), 2.67 (m, 2H), 2.49 (m, 1H), 2.03 (m, 1H), 1.92-1.61 (m, 2H), 1.56 (m, 1H), 1.36 (m, 2H), 0.96 (t, J=7.1 Hz, 3H).

### Example 7

### 2-(2-Phenylethyl)pentanoic acid

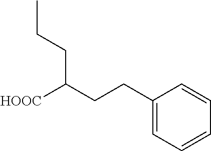

This compound was prepared following the same procedure than the one followed in Example 3, using as starting ester the methyl 2-(2-phenylethyl)pentanoate, to furnish the compound as a colourless oil after flash chromatography purification (yield: 43%).

¹H NMR (CDCl₃, 250 MHz) δ ppm: 7.31-7.15 (m, 5H), 2.65 (m, 2H), 2.43 (m, 1H), 1.97 (m, 1H), 1.86-1.58 (m, 2H), 1.55-1.23 (m, 3H), 0.91 (t, J=7.1 Hz, 3H).

### Example 8

### Methyl 2-benzyl-4-phenylbutanoate

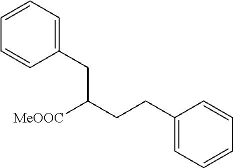

This compound was prepared following the same procedure than the one followed in Example 2, using BnBr as alkylating reagent instead of MeI. Flash chromatography purification afforded the compound as a colourless oil (yield: 82%).

¹H NMR (CDCl₃, 250 MHz) δ ppm: 7.32-7.07 (m, 10H), 3.61 (s, 3H), 2.97 (m, 1H), 2.82-2.53 (m, 4H), 1.98 (m, 1H), 1.81 (m, 1H).

### Example 9

### 2-Benzyl-4-phenylbutanoic acid

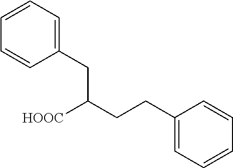

This compound was prepared following the same procedure than the one described in Example 3, using as starting ester the methyl 2-benzyl-4-phenylbutanoate to furnish the compound as a colourless oil after flash chromatography purification (yield: 56%).

¹H NMR (CDCl₃, 250 MHz) δ ppm: 7.61-7.36 (m, 10H), 3.30 (m, 1H), 3.13-2.79 (m, 4H), 2.27 (m, 1H), 2.09 (m, 1H).

### Example 10

### Methyl 5-phenyl-2-(2-phenylethyl)pentanoate

This compound was prepared following the same procedure than the one followed in Example 2, using PhCH₂CH₂CH₂Br as alkylating reagent instead of MeI. Flash chromatography purification afforded the compound as a colourless oil (yield: 20%).

¹H NMR (CDCl₃, 250 MHz) δ ppm: 7.35-7.10 (m, 10H), 3.67 (s, 3H), 2.57 (m, 3H), 2.24 (m, 2H), 1.93-1.81 (m, 4H), 1.19 (m, 2H).

### Example 11

### 5-Phenyl-2-(2-phenylethyl)pentanoic acid

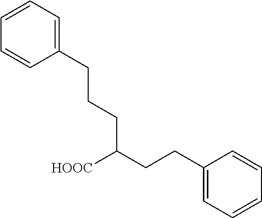

This compound was prepared following the same procedure than the one followed in Example 3, using as starting ester the methyl 5-phenyl-2-(2-phenylethyl)pentanoate, to furnish the compound as a colourless oil after flash chromatography purification (yield: 13%).

¹H NMR (CDCl₃, 250 MHz) δ ppm: 7.52-7.27 (m, 10H), 2.78 (m, 4H), 2.60 (m, 1H), 2.15 (m, 1H), 2.04-1.66 (m, 5H).

### Example 12

### 2-{4-[(methylamino)sulfonyl]benzyl}-4-phenylbutanoic acid

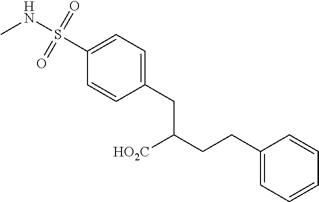

### a) Synthesis of N,4-dimethylbenzenesulfonamide

MeNH₂ (8 ml, 8 M solution in EtOH, 64 mmol) was added to a solution of 4-methylbenzenesulfonyl chloride (4.72 g, 24.757 mmol) in THF (100 ml). The reaction mixture was stirred at room temperature for 5 min, poured into H₂O (400 ml) and extracted with CH₂Cl₂ (500 ml). The organic layer was dried over Na₂SO₄ (anhydrous), filtered and concentrated, to furnish N,4-dimethylbenzenesulfon-amide, that was submitted to next step without further purification (4.27 g, white solid, yield: 93%).

¹H NMR (CDCl₃, 250 MHz) δ ppm: 7.74 (d, J=8.2 Hz, 2H), 7.31 (d, J=8.2 Hz, 2H), 4.66 (c, J=5.5 Hz, 1H), 2.63 (d, J=5.5. Hz, 3H), 2.42 (s, 3H).

### b) Synthesis of tert-butyl methyl[(4-methylphenyl)sulfonyl]carbamate

Boc₂O (2.82 g, 12.955 mmol) was added to a solution of N,4-dimethyl benzenesulfonamide (2.0 g, 10.796 mmol), DMAP (197 mg, 1.619 mmol) and DIPEA (5.5 ml, 32.38 mmol) in CH₃CN (80 ml). The reaction mixture was stirred at room temperature for 30 min, poured into H₂O (200 ml) and extracted with EtOAc (200 ml). The organic layer was dried over Na₂SO₄ (anhydrous), filtered and concentrated. The crude residue was flash chromatographed on SiO₂ (10→20% EtOAc/hexanes), to afford tert-butyl methyl[(4-methylphenyl)sulfonyl]carbamate (3.07 g, white solid, yield: 99%).

¹H NMR (CDCl₃, 250 MHz) δ ppm: 7.77 (d, J=8.3 Hz, 2H), 7.30 (d, J=8.3 Hz, 2H), 3.34 (s, 3H), 2.44 (s, 3H), 1.34 (s, 9H).

### c) Synthesis of tert-butyl[4-(bromomethyl)phenyl]sulfonyl(methyl)carbamate

NBS (1.03 g, 5.786 mmol) was added to a solution of tert-butyl[4-(bromomethyl)phenyl]sulfonyl(methyl)carbamate (1.50 g, 5.256 mmol) in CCl₄ (40 ml). The reaction mixture was warmed up to reflux, and allowed to react for 6 h. Solvent was concentrated off, and the crude residue was flash chromatographed on SiO₂ (10→30% EtOAc/hexanes), to afford tert-butyl[4-(bromomethyl)phenyl]sulfonyl(methyl)carbamate (2.0 g, pale yellow-coloured oil, yield: 100%. Purity: 60%). Mixture of starting material and product, which was submitted to next step without further purification.

¹H NMR (CDCl₃, 250 MHz) δ ppm: 7.86 (d, J=8.5 Hz, 2H), 7.53 (d, J=8.5 Hz, 2H), 4.50 (s, 2H), 3.35 (s, 3H), 1.35 (s, 9H).

### d) Synthesis of 2-{4-[tert-butyl(methylamino)sulfonyl]benzyl}-4-phenylbutanoic acid

The compound was prepared following the same procedure than the one of Example 2, using tert-butyl[4-(bromomethyl)phenyl]sulfonyl(methyl) carbamate as alkylating reagent instead of MeI. Flash chromatography purification afforded the compound as a colourless oil (yield: 36%).

¹H NMR (CDCl₃, 250 MHz) δ ppm: 7.78 (d, J=8.5 Hz, 2H), 7.30-7.10 (m, 7H), 3.60 (s, 3H), 3.34 (s, 3H), 3.04 (m, 1H), 2.91-2.83 (m, 4H), 1.98 (m, 1H), 1.82 (m, 1H), 1.32 (s, 9H).

### e) Obtaining of the title compound 2-{4-[(methylamino)sulfonyl]benzyl}-4-phenylbutanoic acid

Starting from the product resulting from step (d), it was followed the same process than the one followed in Example 3, to furnish the compound as a white solid after flash chromatography purification (yield: 62%).

¹H NMR (CDCl₃, 250 MHz) ppm: 7.74 (d, J=8.2 Hz, 2H), 7.34-7.10 (m, 7H), 5.14 (bs, 1H), 3.09-2.83 (m, 2H), 2.74-2.48 (m, 6H), 2.03 (m, 1H), 1.82 (m, 1H).

### Example 13

### 5-(4-[(Methylamino)sulfonyl]phenyl)-2-(2-phenylethyl)pentanoic acid

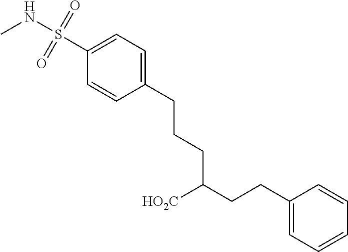

### a) Synthesis of 4-Bromo-N-methylbenzenesulfonamide

MeNH₂ (7.5 ml, 8 M solution in EtOH, 60 mmol) was added to a solution of 4-bromobenzenesulfonyl chloride (5.0 g, 19.568 mmol) in THF (120 ml). The reaction mixture was stirred at room temperature for 5 min, poured into NH₄Cl (saturated aqueous solution, 300 ml) and extracted with EtOAc (500 ml). The organic layer was dried over Na₂SO₄ (anhydrous), filtered and concentrated, to furnish 4-bromo-N-methylbenzenesulfonamide, which was submitted to next step without further purification (4.60 g, white solid, yield: 94%).

¹H NMR (CDCl₃, 250 MHz) δ ppm: 7.70 (m, 4H), 4.49 (bs, 1H), 2.67 (d, J=5.2 Hz, 3H).

### b) Synthesis of tert-butyl (4-bromophenyl)sulfonyl(methyl)carbamate

Boc₂O (4.711 g, 21.589 mmol) was added to a solution of 4-bromo-N-methylbenzenesulfonamide (4.5 g, 17.99 mmol), DMAP (329 mg, 2.698 mmol) and DIPEA (9.25 ml, 53.97 mmol) in CH₃CN (100 ml). The reaction mixture was stirred at room temperature for 30 min, poured into H₂O (200 ml) and extracted with EtOAc (200 ml). The organic layer was dried over Na₂SO₄ (anhydrous), filtered and concentrated. The crude residue was flash chromatographed on SiO₂ (10→20% EtOAc/hexanes), to afford tert-butyl (4-bromophenyl)sulfonyl(methyl)carbamate (6.18 g, white solid, yield: 98%).

¹H NMR (CDCl₃, 250 MHz) δ ppm: 7.77 (d, J=9.0 Hz, 2H), 7.66 (d, J=9.0 Hz, 2H), 3.34 (s, 3H), 1.37 (s, 9H).

### c) Synthesis of tert-butyl[4-(3-hydroxyprop-1-ynyl)phenyl]sulfonyl(methyl) carbamate. (Method C: Sonogashira coupling with prop-2-yn-1-ol)

A solution of prop-2-yn-1-ol (949 mg, 16.939 mmol) in THF (6 ml) was dropwise added to a refluxing suspension of tert-butyl (4-bromophenyl) sulfonyl(methyl)carbamate (6.18 g, 17.645 mmol), CuI (152 mg, 0.794 mmol) and PdCl₂(PPh₃)₂ (557 mg, 0.794 mmol) in a mixture of Et₃N (49 ml) and THF (100 ml). The reaction mixture was refluxed for 3 h, and then allowed to reach room temperature Solvent was concentrated off, and the crude residue was flash chromatographed on SiO₂ (0→10% EtOAc/hexanes), to afford tert-butyl [4-(3-hydroxyprop-1-ynyl)phenyl]sulfonyl(methyl)carbamate (4.48 g, pale yellow-coloured oil, yield: 78%).

¹H NMR (CDCl₃, 250 MHz) δ ppm: 7.82 (d, J=8.8 Hz, 2H), 7.53 (d, J=8.8 Hz, 2H), 4.53 (s, 2H), 3.35 (s, 3H), 1.99 (bs, 1H), 1.35 (s, 9H).

### d) Synthesis of tert-butyl[4-(3-bromoprop-1-ynyl)phenyl]sulfonyl(methyl) carbamate. (Method D: alkynol bromination)

CBr₄ (1.172 g, 3.534 mmol) was added to a −18° C. cooled solution of tert-butyl [4-(3-hydroxyprop-1-ynyl)phenyl]sulfonyl(methyl)carbamate (1.0 g, 3.073 mmol) and PPh₃ (967 mg, 3.687 mmol) in CH₂Cl₂ (40 ml). The reaction mixture was allowed to react at low temperature for 30 min, poured into H₂O (200 ml), and extracted with CH₂Cl₂ (200 ml). The organic layer was dried over Na₂SO₄ (anhydrous), filtered and concentrated. The crude residue was flash chromatographed on SiO₂ (50% EtOAc/hexanes), to afford tert-butyl[4-(3-bromoprop-1-ynyl)phenyl]sulfonyl(methyl)carbamate (1.03 g, pale coloured solid, yield: 86%).

¹H NMR (CDCl₃, 250 MHz) δ ppm: 7.85 (d, J=8.5 Hz), 7.55 (d, J=8.5 Hz), 4.16 (s, 2H), 3.34 (s, 3H), 1.33 (s, 9H).

### e) Synthesis of methyl 5-{4-[tert-butyl(methylamino)sulfonyl]phenyl}-2-(2-phenylethyl)pent-4-ynoate

The resulting product of step (d) was submitted to the procedure described in Example 2, using tert-butyl[4-(3-bromoprop-1-ynyl)phenyl]sulfonyl(methyl)carbamate as alkylating reagent instead of MeI. Flash chromatography purification afforded the compound as a pale yellow-coloured solid (yield: 26%).

¹H NMR (CDCl₃, 250 MHz) δ ppm: 7.79 (d, J=8.5 Hz, 2H), 7.47 (d, J=8.5 Hz, 2H), 7.34-7.16 (m, 5H), 3.75 (s, 3H), 3.35 (s, 3H), 2.70 (m, 5H), 2.08 (m, 2H), 1.36 (s, 9H).

### f) Synthesis of 5-(4-[tert-butyl(methylamino)sulfonyl]phenyl)-2-(2-phenylethyl) pentanoic acid. (Method E: Hydrogenation)

A suspension of methyl 5-{4-[tert-butyl(methylamino)sulfonyl]phenyl}-2-(2-phenylethyl)pent-4-ynoate (211 mg, 0.434 mmol) and Pd/C (69 mg, 10% palladium on activated carbon, 0.065 mmol) in MeOH (15 ml) was stirred under H₂ atmosphere (balloon) for 1 h. It was filtered through Celite, washed with MeOH, and concentrated, to furnish methyl 5-{4-[tert-butyl(methylamino) sulfonyl]phenyl}-2-(2-phenylethyl)pent-4-ynoate (195 mg, pale yellow-coloured oil, yield: 91%). The crude residue was submitted to next step without purification.

¹H NMR (CDCl₃, 250 MHz) δ ppm: 7.63 (d, J=8.6 Hz, 2H), 7.14 (m, 5H), 7.00 (d, J=8.6 Hz, 2H), 3.53 (s, 3H), 3.19 (s, 3H), 2.58-2.11 (m, 5H), 2.02-1.65 (m, 2H), 1.60-1.35 (m, 4H), 1.20 (s, 9H).

### g) Obtaining of the title compound 5-(4-[(Methylamino)sulfonyl]phenyl)-2-(2-phenylethyl)pentanoic acid

Starting from the product resulting in step (f), it was followed the same procedure than the one described in Example 3, to furnish the compound as a colourless oil after flash chromatography purification (yield: 37%).

¹H NMR (CDCl₃, 250 MHz) δ ppm: 7.70 (d, J=8.2 Hz, 2H), 7.22 (m, 4H), 7.12 (m, 3H), 4.71/bs, 1H), 2.58 (s, 3H), 2.68-2.49 (m, 4H), 2.36 (m, 1H), 1.93 (m, 1H), 1.78-1.41 (m, 5H).

### Example 14

### 6-Phenyl-2-(2-phenylethyl)hexanoic acid

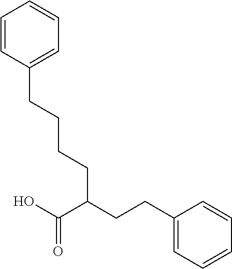

### a) Methyl 6-phenyl-2-(2-phenylethyl)hexanoate

The compound was synthesized from methyl-4-phenylbutanoate and (4-iodobutyl)benzene following the experimental procedure detailed in Method A. It was purified by flash chromatography on SiO₂ (0→4% EtOAc/hexanes) to furnish a colourless oil (yield: 88%).

¹H NMR (CDCl₃, 250 MHz) δ ppm: 7.36-7.09 (m, 10H), 3.67 (s, 3H), 2.58 (m, 4H), 2.39 (m, 1H), 2.00-1.43 (m, 6H), 1.39 (m, 2H).

### b) 6-Phenyl-2-(2-phenylethyl)hexanoic acid

The compound was synthesized from methyl 6-phenyl-2-(2-phenylethyl)hexanoate following the experimental procedure detailed in Method B. The crude residue was purified by flash chromatography on SiO₂ (5→25% EtOAc/hexanes) to furnish a colourless oil (yield: 70%).

¹H NMR (CDCl₃, 250 MHz) δ ppm: 7.29-7.08 (m, 10H), 2.58 (m, 4H), 2.41 (m, 1H), 1.96 (m, 1H), 1.83-1.46 (m, 5H), 1.35 (m, 2H). EI MS: m/z=295 (M−1).

### Example 15

### (Acetyloxy)methyl 2-benzyl-4-phenylbutanoate

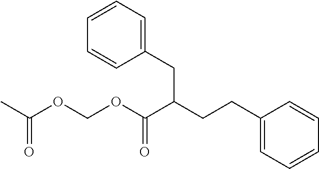

Bromomethyl acetate (0.125 mL, 1.274 mmol) was added to a solution of 2-benzyl-4-phenylbutanoic acid (Example 9) (300 mg, 1.179 mmol) and DIPEA (0.25 mL, 1.46 mmol) in CH₃CN (15 mL). The reaction mixture was stirred at r.t. for 30 min, poured into H₂O (100 mL) and extracted with EtOAc (100 mL). The organic layer was washed with HCl (100 mL, 1% aqueous solution), NaHCO₃ (100 mL, saturated aqueous solution), dried over Na₂SO₄ (anhydrous), filtered and concentrated. The crude residue was flash chromatographed on SiO₂ (5→20% EtOAc/hexanes), to afford 200 mg of (acetyloxy)methyl 2-benzyl-4-phenylbutanoate (colourless oil, yield: 52%).

¹H NMR (CDCl₃, 250 MHz) δ ppm: 7.29 (m, 6H), 7.20 (m, 4H), 5.49 (m, 2H), 3.02 (m, 1H), 2.83 (m, 2H), 2.66 (m, 2H), 1.86 (s, 3H), 1.84-1.56 (m, 2H).

EI MS: m/z=327 (M+1), 344 (M+18).

### Example 16

### (Acetyloxy)methyl 5-phenyl-2-(2-phenylethyl)pentanoate

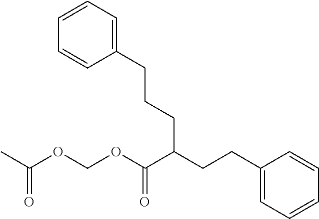

Bromomethyl acetate (0.2 mL, 2.039 mmol) was added to a solution of 5-phenyl-2-(2-phenylethyl)pentanoic acid (Example 11) (514 mg, 1.82 mmol) and DIPEA (0.39 mL, 2.275 mmol) in CH₃CN (25 mL). The reaction mixture was stirred at r.t. for 1 h and then it was poured into H₂O (40 mL) and extracted with EtOAc (2×50 mL). The organic layer was dried over Na₂SO₄ (anhydrous), filtered and concentrated. The crude residue was flash chromatographed on SiO₂ (5→10% EtOAc/hexanes), to afford 224 mg of (acetyloxy)methyl 5-phenyl-2-(2-phenylethyl)pentanoate (yellow coloured oil, yield: 35%).

¹H NMR (CDCl₃, 250 MHz) δ ppm: 7.32-7.24 (m, 4H), 7.23-7.11 (m, 6H), 5.77 (s, 2H), 2.68-2.47 (m, 6H), 2.07 (s, 3H), 1.85-1.52 (m, 5H).

EI MS: m/z=355 (M+1).

### Example 17

### (Acetyloxy)methyl 5-(3-{[(3,4-dimethoxy phenyl)amino]sulfonyl}phenyl)-2-(2-phenylethyl)pentanoate

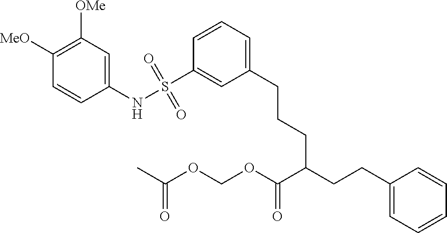

Bromomethyl acetate (0.1 mL, 1.019 mmol) was added to a solution of 5-(3-{[(3,4-dimethoxyphenyl)amino]sulfonyl}phenyl)-2-(2-phenylethyl)pentanoic acid (Example 43) (370 mg, 0.743 mmol) and DIPEA (0.2 mL, 1.168 mmol) in CH₃CN (25 mL). The reaction mixture was stirred at r.t. for 4 h and solvent was concentrated off. The crude residue was flash chromatographed on SiO₂ (10 40% EtOAc/hexanes), to afford 374 mg of (acetyloxy)methyl 5-(3-{[(3,4-dimethoxyphenyl)amino]sulfonyl}phenyl)-2-(2-phenylethyl)pentanoate (colourless oil, yield: 88%).

¹H NMR (CDCl₃, 250 MHz) δ ppm: 7.59 (m, 2H), 7.32 (m, 5H), 7.21 (m, 3H), 6.82-6.58 (m, 3H), 5.81 (m, 2H), 3.83 (s, 3H), 3.79 (s, 3H), 2.63 (m, 4H), 2.45 (m, 1H), 2.15 (s, 3H), 2.00 (m, 1H), 1.84-1.46 (m, 5H).

EI MS: m/z=570 (M+1), 587 (M+18).

### Example 18

### (Acetyloxy)methyl 5-(3-[(4-methylanilino sulfonyl]phenyl)-2-(2-phenylethyl)pentanoate

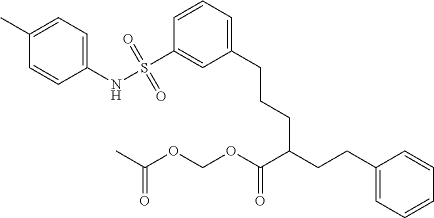

Bromomethyl acetate (116 mg, 0.758 mmol) was added to a solution of 5-(3-[(4-methylanilinosulfonyl]phenyl)-2-(2-phenylethyl)pentanoic acid (Example 36) (250 mg, 0.554 mmol) and DIPEA (0.15 mL, 0.869 mmol) in CH₃CN (20 mL). The reaction mixture was stirred at r.t. for 4 h and solvent was concentrated off. The crude residue was flash chromatographed on SiO₂ (5→20% EtOAc/hexanes), to afford 122 mg of (acetyloxy)methyl 5-(3-[(4-methylanilino sulfonyl]phenyl)-2-(2-phenylethyl)pentanoate (colourless oil, yield: 42%).

¹H NMR (CDCl₃, 250 MHz) δ ppm: 7.57-7.47 (m, 2H), 7.34-7.24 (m, 5H), 7.16 (t, J=7.1 Hz, 2H), 6.98 (m, 4H), 6.68 (s, 1H), 5.77 (dd, J=7.9 Hz, 5.5 Hz, 2H), 2.67-2.30 (m, 5H), 2.25 (s, 3H), 2.11 (s, 3H), 1.63-1.47 (m, 6H).

EI MS: m/z=524 (M+1), 541 (M+18).

### Example 19

### (Acetyloxy)methyl 5-(3-[(methylamino)sulfonyl]phenyl)-2-(2-phenylethyl)pentanoate

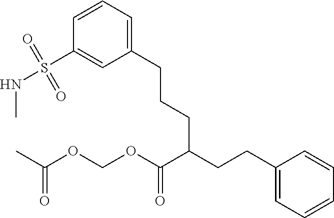

Bromomethyl acetate (0.09 mL, 0.917 mmol) was added to a solution of 5-(3-[(methylamino)sulfonyl]phenyl)-2-(2-phenylethyl)pentanoic acid (Example 41) (300 mg, 0.789 mmol) and DIPEA (0.2 mL, 1.168 mmol) in CH₃CN (20 mL). The reaction mixture was stirred at r.t. for 30 min, it was poured into H₂O (100 mL) and extracted with EtOAc (100 mL). The organic layer was washed with HCl (60 mL, 2% aqueous solution), NaHCO₃ (60 mL, saturated aqueous solution), dried over Na₂SO₄ (anhydrous), filtered and concentrated. The crude residue was flash chromatographed on SiO₂ (20→100% EtOAc/hexanes), to afford 120 mg of (acetyloxy)methyl 5-(3-[(methylamino)sulfonyl]phenyl)-2-(2-phenylethyl)pentanoate (colourless oil, yield: 34%).

¹H NMR (CDCl₃, 250 MHz) δ ppm: 7.70 (m, 2H), 7.52-7.26 (m, 4H), 7.25-7.16 (m, 3H), 5.85 (dd, J=5.5 Hz, 2.5 Hz, 2H), 4.78 (m, 1H), 2.90 (d, J=5.5 Hz, 3H), 2.67-2.41 (m, 5H), 2.19 (s, 3H), 2.01 (m, 1H), 1.89-1.55 (m, 5H).

EI MS: m/z=448 (M+1), 465 (M+18).

### Example 20

### Sodium 5-phenyl-2-(2-phenylethyl)pentanoate

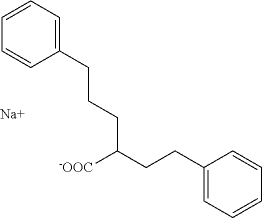

NaH (25 mg, 60% mineral oil suspension, 0.64 mmol) was added to a solution of 5-phenyl-2-(2-phenylethyl)pentanoic acid (Example 11) (200 mg, 0.708 mmol) in THF (10 mL). The reaction mixture was stirred at r.t. for 15 min, and solvent was concentrated off. The crude residue was slurred with Et₂O (2×5 mL) and hexanes (5 mL), to give 95 mg of sodium 5-phenyl-2-(2-phenylethyl) pentanoate (white solid, yield: 45%).

¹H NMR (MeOD, 250 MHz) δ ppm: 7.38-7.17 (m, 10H), 2.70 (m, 4H), 2.42 (m, 1H), 1.95 (m, 1H), 1.74 (m, 4H), 1.55 (m, 1H).

EI MS: m/z=281 (M−18).

### Example 21

### (4E)-5-Phenyl-2-(2-phenylethyl)pent-4-enoic acid

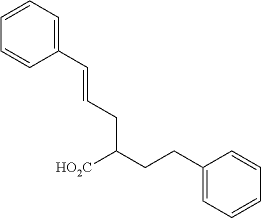

### a) Methyl (4E)-5-phenyl-2-(2-phenylethyl)pent-4-enoate

The compound was prepared from methyl 4-phenylbutanoate following the general procedure A, by using [(1E)-3-bromoprop-1-enyl]benzene as alkylating reagent. The crude residue was flash chromatographed on SiO₂ (0→2% EtOAc/hexanes), to afford the compound as a yellow-coloured oil (yield: 41%).

¹H NMR (CDCl₃, 250 MHz) δ ppm: 7.36-7.14 (m, 10H), 6.41 (d, J=15.6 Hz, 1H), 6.11 (m, 1H), 3.69 (s, 3H), 2.74-2.28 (m, 5H), 2.11-1.75 (m, 2H).

### b) (4E)-5-Phenyl-2-(2-phenylethyl)pent-4-enoic acid

The compound was synthesized from methyl (4E)-5-phenyl-2-(2-phenylethyl)pent-4-enoate following the experimental procedure detailed in Method B. It was purified by flash chromatography on SiO₂ (5 10% EtOAc/hexanes) to furnish a white solid (yield: 79%).

¹H NMR (CDCl₃, 250 MHz) ppm: 7.39-7.12 (m, 10H), 6.44 (d, J=15.6 Hz, 1H), 6.14 (m, 1H), 2.80-2.29 (m, 5H), 2.10-1.78 (m, 2H).

### Example 22

### 5-(2-Naphthyl)-2-(2-phenylethyl)pentanoic acid

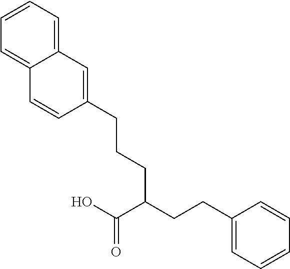

### a) 3-(2-Naphthyl)prop-2-yn-1-ol

The compound was synthesized from 2-bromonaphthalene following the experimental procedure detailed in Method C. It was purified by flash chromatography on SiO₂ (10→40% EtOAc/hexanes) to furnish a brown coloured solid (yield: 98%).

¹H NMR (CDCl₃, 250 MHz) δ ppm: 7.97 (s, 1H), 7.80 (m, 3H), 7.50 (m, 3H), 4.56 (s, 2H), 2.15 (bs, 1H).

### b) 2-(3-Bromoprop-1-ynyl)naphthalene

The compound was synthesized from 3-(2-naphthyl)prop-2-yn-1-ol following the experimental procedure detailed in Method D. It was purified by flash chromatography on SiO₂ (0→10% EtOAc/hexanes) to furnish a brown coloured solid (yield: 96%).

¹H NMR (CDCl₃, 250 MHz) δ ppm: 7.99 (s, 1H), 7.80 (m, 3H), 7.50 (m, 3H), 4.23 (s, 2H).

### c) Methyl 5-(2-naphthyl)-2-(2-phenylethyl)pent-4-ynoate

The compound was synthesized from 2-(3-bromoprop-1-ynyl)naphthalene and methyl 4-phenylbutanoate following the experimental procedure detailed in Method A. It was purified by flash chromatography on SiO₂ (10→15% EtOAc/hexanes) to furnish an orange coloured solid (yield: 76%).

¹H NMR (CDCl₃, 250 MHz) δ ppm: 7.91 (s, 1H), 7.79 (m, 3H), 7.47 (m, 3H), 7.37-7.17 (m, 5H), 3.77 (s, 3H), 2.88-2.61 (m, 5H), 2.14 (m, 2H).

### d) Methyl 2-(2-phenylethyl)-5-(2-naphthyl)pentanoate

The compound was synthesized from methyl 5-(2-naphthyl)-2-(2-phenylethyl)pent-4-ynoate following the experimental procedure detailed in Method E. It was purified by flash chromatography on SiO₂ (10→15% EtOAc/hexanes) to furnish a colourless oil (yield: 80%).

¹H NMR (CDCl₃, 250 MHz) δ ppm: 7.80 (m, 3H), 7.61 (s, 1H), 7.45 (m, 2H), 7.30 (m, 3H), 7.19 (m, 3H), 3.71 (s, 3H), 2.79 (t, J=7.4 Hz, 2H), 2.66-2.34 (m, 3H), 1.97 (m, 1H), 1.86-1.53 (m, 5H).

### e) 5-(2-Naphthyl)-2-(2-phenylethyl)pentanoic acid

The compound was synthesized from methyl 2-(2-phenylethyl)-5-(2-naphthyl)pentanoate following the experimental procedure detailed in Method B. It was purified by flash chromatography on SiO₂ (10→15% EtOAc/hexanes) to furnish a white solid (yield: 35%).

¹H NMR (CDCl₃, 250 MHz) δ ppm: 7.78 (m, 3H), 7.60 (s, 1H), 7.44 (m, 2H), 7.28 (m, 3H), 7.19 (m, 3H), 2.84-2.39 (m, 5H), 2.01 (m, 1H), 1.87-1.54 (m, 5H).

EI MS: m/z=350 (M+18).

### Example 23

### 5-(1-Naphthyl)-2-(2-phenylethyl)pentanoic acid

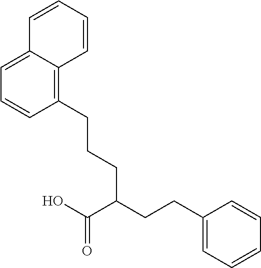

### a) 3-(1-Naphthyl)prop-2-yn-1-ol

The compound was synthesized from 1-bromonaphthalene following the experimental procedure detailed in Method C. It was purified by flash chromatography on SiO₂ (10→30% EtOAc/hexanes) to furnish a yellow coloured oil (yield: 55%).

¹H NMR (CDCl₃, 250 MHz) δ ppm: 8.33 (d, J=6.8 Hz, 1H), 7.85 (m, 3H), 7.68 (d, J=6.8 Hz, 1H), 7.54 (m, 3H), 7.42 (m, 1H), 4.66 (s, 2H), 2.04 (bs, 1H).

### b) 1-(3-Bromoprop-1-ynyl)naphthalene

The compound was synthesized from 3-(1-naphthyl)prop-2-yn-1-ol following the experimental procedure detailed in Method D. It was purified by flash chromatography on SiO₂ (0→10% EtOAc/hexanes) to furnish a yellow coloured oil (yield: 82%).

¹H NMR (CDCl₃, 250 MHz) δ ppm: 8.31 (dd, J=1.1 Hz, 8.2 Hz, 1H), 7.85 (dd, J=0.8 Hz, 7.1 Hz, 2H), 7.68 (dd, J=1.1 Hz, 7.1 Hz, 1H), 7.63-7.49 (m, 2H), 7.43 (dd, J=7.1 Hz, 8.2 Hz, 1H), 4.34 (s, 2H).

### c) Methyl 5-(1-naphthyl)-2-(2-phenylethyl)pent-4-ynoate

The compound was synthesized from 1-(3-bromoprop-1-ynyl)naphthalene and methyl 4-phenylbutanoate following the experimental procedure detailed in Method A. It was purified by flash chromatography on SiO₂ (5→10% EtOAc/hexanes) to furnish a yellow coloured oil (yield: 60%).

¹H NMR (CDCl₃, 250 MHz) δ ppm: 8.34 (m, 1H), 7.87 (m, 2H), 7.69-7.53 (m, 3H), 7.45 (t, J=7.1 Hz, 1H), 7.37-7.21 (m, 5H), 3.81 (s, 3H), 2.94-2.54 (m, 5H), 2.21 (m, 2H).

### d) Methyl 5-(1-naphthyl)-2-(2-phenylethyl)pentanoate

The compound was synthesized from methyl 5-(1-naphthyl)-2-(2-phenylethyl)pent-4-ynoate following the experimental procedure detailed in Method E. It was purified by flash chromatography on SiO₂ (5→10% EtOAc/hexanes) to furnish a yellow coloured oil (yield: 79%).

¹H NMR (CDCl₃, 250 MHz) δ ppm: 8.05 (m, 1H), 7.89 (m, 1H), 7.75 (m, 1H), 7.53 (m, 2H), 7.45 (m, 1H), 7.36-7.14 (m, 6H), 3.72 (s, 3H), 3.10 (t, J=6.8 Hz, 2H), 2.70-2.46 (m, 3H), 2.10-1.68 (m, 6H).

### e) 5-(1-Naphthyl)-2-(2-phenylethyl)pentanoic acid

The compound was synthesized from methyl 5-(1-naphthyl)-2-(2-phenylethyl)pentanoate, following the experimental procedure detailed in Method B. It was purified by flash chromatography on SiO₂ (10 15% EtOAc/hexanes) to yield a white solid (yield: 35%).

¹H NMR (CDCl₃, 250 MHz) δ ppm: 8.05 (m, 1H), 7.90 (m, 1H), 7.76 (m, 1H), 7.59-7.38 (m, 3H), 7.36-7.15 (m, 6H), 3.12 (t, J=7.4 Hz, 2H), 2.81-2.45 (m, 3H), 2.12-1.64 (m, 6H).

EI MS: m/z=331 (M−1).

### Example 24

### 5-[3-{[4-(dimethylamino)benzoyl]amino}phenyl]-2-(2-phenylethyl)pentanoic acid hydrochloride

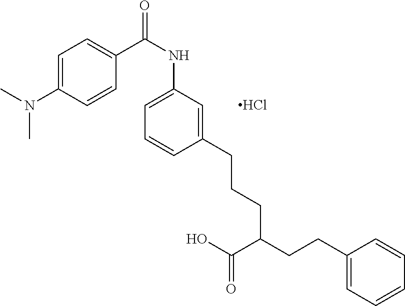

### a) N,N-Dibenzyl-N-(3-bromophenyl)amine

BnBr (7 mL, 58.524 mmol) was added to a suspension of K₂CO₃ (7 g, 50.647 mmol) and 3-bromoaniline (3.0 g, 17.438 mmol) in CH₃CN (100 mL). The reaction mixture was warmed up to reflux and stirred for 4 h. It was allowed to reach r.t. poured into H₂O (300 mL) and extracted with EtOAc (300 mL). The organic layer was dried over Na₂SO₄ (anhydrous), filtered and concentrated.

The crude residue was flash chromatographed on SiO₂ (0→5% EtOAc/hexanes) to furnish 6.0 g of N,N-Dibenzyl-N-(3-bromophenyl)amine (colourless oil, yield: 97%).

EI MS: m/z=353 (M+1).

### b) 3-[3-(Dibenzylamino)phenyl]prop-2-yn-1-ol

The compound was synthesized from N,N-Dibenzyl-N-(3-bromophenyl)amine following the experimental procedure detailed in Method C. It was purified by flash chromatography on SiO₂ (10→20% EtOAc/hexanes) to yield a colourless oil (yield: 67%).

¹H NMR (CDCl₃, 250 MHz) δ ppm: 7.88-7.65 (m, 11H), 7.58 (m, 1H), 7.30 (m, 2H), 7.18 (dd, J=8.2 Hz, 2.4 Hz, 1H), 5.13 (s, 4H), 4.92 (s, 2H).

### c) N,N-Dibenzyl-N-[3-(3-bromoprop-1-ynyl)phenyl]amine

The compound was synthesized from 3-[3-(dibenzylamino)phenyl]prop-2-yn-1-ol following the experimental procedure detailed in Method D. It was purified by flash chromatography on SiO₂ (10→20% EtOAc/hexanes) to yield a yellow-coloured oil (yield: 87%).

¹H NMR (CDCl₃, 250 MHz) δ ppm: 7.86-7.65 (m, 11H), 7.58 (t, J=7.9 Hz, 1H), 7.33-7.18 (m, 3H), 5.11 (s, 4H), 4.60 (s, 2H).

### d) Methyl 5-(3-N,N-dibenzylaminophenyl)-2-(2-phenylethyl)pent-4-ynoate

The compound was synthesized from methyl 4-phenylbutanoate and N,N-dibenzyl-N-[3-(3-bromoprop-1-ynyl)phenyl]amine following the experimental procedure detailed in Method A. It was purified by flash chromatography on SiO₂ (5% EtOAc/hexanes) to yield a yellow-coloured oil (yield: 65%).

¹H NMR (CDCl₃, 250 MHz) δ ppm: 7.41-7.12 (m, 15H), 7.06 (t, J=8.2 Hz, 1H), 6.79-6.61 (m, 3H), 4.62 (s, 4H), 3.64 (s, 3H), 2.64 (m, 5H), 1.89 (m, 1H).

### e) Methyl 5-(3-aminophenyl)-2-(2-phenylethyl)pentanoate

The compound was synthesized from methyl 5-(3-N,N-dibenzylaminophenyl)-2-(2-phenylethyl)pent-4-ynoate following the experimental procedure detailed in Method E. It was purified by flash chromatography on SiO₂ (20→60% EtOAc/hexanes) to yield an orange-coloured oil (yield: 40%).

¹H NMR (CDCl₃, 250 MHz) δ ppm: 7.32-7.12 (m, 7H), 7.06 (t, J=7.6 Hz, 1H), 6.53 (m, 3H), 3.68 (s, 3H), 2.63-2.35 (m, 5H), 2.03-1.43 (m, 6H).

### f) Methyl 5-[3-{[4-(dimethylamino)benzoyl]amino}phenyl]-2-(2-phenylethyl)pentanoate

4-(Dimethylamino)benzoyl chloride (320 mg, 1.742 mmol) was added to a solution of methyl 5-(3-aminophenyl)-2-(2-phenylethyl)pentanoate (450 mg, 1.444 mmol), DIPEA (0.8 mL, 4.673 mmol) and DMAP (30 mg, 0.246 mmol) in CH₂Cl₂ (30 mL). The reaction mixture was stirred at r.t. for 1 h, poured into H₂O (200 mL), and extracted with CH₂Cl₂ (100 mL). The organic layer was dried over Na₂SO₄ (anhydrous), filtered and concentrated. The crude residue was flash chromatographed on SiO₂ (10→40% EtOAc/hexanes) to furnish 642 mg of methyl 5-[3-{[4-(dimethylamino)benzoyl]amino}phenyl]-2-(2-phenylethyl)pentanoate (orange-coloured oil, yield: 97%).

¹H NMR (CDCl₃, 250 MHz) δ ppm: 7.78 (d, J=9.0 Hz, 2H), 7.67 (bs, 1H), 7.44 (m, 2H), 7.31-7.12 (m, 5H), 6.89 (m, 1H), 6.71 (d, J=9.0 Hz, 2H), 3.68 (s, 3H), 3.05 (s, 6H), 2.64-2.35 (m, 5H), 2.02-1.46 (m, 6H).

### g) 5-[3-{[4-(Dimethylamino)benzoyl]amino}phenyl]-2-(2-phenylethyl)pentanoic acid

The compound was synthesized from methyl 5-[3-{[4-(dimethylamino)benzoyl]amino}phenyl]-2-(2-phenylethyl)pentanoate following the experimental procedure detailed in Method B. It was purified by flash chromatography on SiO₂ (10→50% EtOAc/hexanes) to yield a colourless oil (yield: 95%).

¹H NMR (CDCl₃, 250 MHz) δ ppm: 7.78 (m, 3H), 7.44 (m, 2H), 7.30-7.12 (m, 5H), 6.89 (m, 1H), 6.69 (d, J=9.0 Hz, 2H), 3.03 (s, 6H), 2.70-2.35 (m, 5H), 2.02-1.59 (m, 6H).

EI MS: m/z=445 (M+1).

### h) 5-[3-{[4-(dimethylamino)benzoyl]amino}phenyl]-2-(2-phenylethyl)pentanoic acid hydrochloride

A suspension of 5-[3-{[4-(Dimethylamino)benzoyl]amino}phenyl]-2-(2-phenylethyl)pentanoic acid (1.373 mmol) in HCl.Et₂O (2 mL, 2 M solution, 4 mmol) was stirred at r.t. for 18 h. Solvent was concentrated off, and the crude residue was slurred with Et₂O (2×15 mL), to give 5-[3-{[4-(dimethylamino)benzoyl]amino}phenyl]-2-(2-phenylethyl)pentanoic acid hydrochloride (white solid, yield: 48%).

¹H NMR (MeOD, 250 MHz) δ ppm: 8.14 (d, J=9.0 Hz, 2H), 7.69 (d, J=9.0 Hz, 2H), 7.28 (m, 3H), 7.18 (m, 3H), 7.01 (d, J=7.6 Hz, 1H), 2.63 (m, 4H), 2.41 (m, 1H), 1.93 (m, 1H), 1.83-1.50 (m, 5H).

EI MS: m/z=445 (M+1-C₁₋).

### Example 25

### 5-(3′-{[(4-Methylphenyl)sulfonyl]amino}phenyl)-2-(2-phenylethyl)pentanoic acid

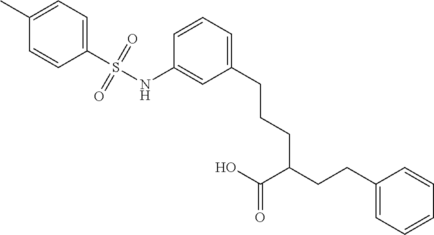

### a) Methyl 5-(3′-{[(4-methylphenyl)sulfonyl]amino}phenyl)-2-(2-phenylethyl)pentanoate

p-TsCl (223 mg, 1.168 mmol) was added to a solution of methyl 5-(3-aminophenyl)-2-(2-phenylethyl)pentanoate (280 mg, 0.899 mmol), DMAP (27 mg, 0.22 mmol) and Et₃N (0.25 mL, 1.79 mmol) in THF (30 mL). The reaction was stirred at r.t. overnight. It was poured into H₂O (80 mL) and extracted with EtOAc (100 mL). The organic layer was dried over Na₂SO₄ (anhydrous), filtered and concentrated. The crude residue was flash chromatographed on SiO₂ (0→5% EtOAc/hexanes) to furnish 270 mg of methyl 5-(3′-{[(4-methylphenyl)sulfonyl]amino}phenyl)-2-(2-phenylethyl)pentanoate (yellow-coloured oil, yield: 65%).

¹H NMR (CDCl₃, 250 MHz) δ ppm: 7.80 (d, J=7.7 Hz, 2H), 7.35-7.12 (m, 11H), 6.84 (m, 1H), 6.76 (bs, 1H), 3.68 (s, 3H), 2.50 (m, 4H), 2.45 (s, 3H), 2.37 (m, 1H), 1.95 (m, 1H), 1.80-1.39 (m, 5H).

### b) 5-(3′-{[(4-Methylphenyl)sulfonyl]amino}phenyl)-2-(2-phenylethyl)pentanoic acid

The compound was synthesized from methyl 5-(3′-{[(4-methylphenyl)sulfonyl]amino}phenyl)-2-(2-phenylethyl)pentanoate following the experimental procedure detailed in Method B. It was purified by flash chromatography on SiO₂ (10→40% EtOAc/hexanes) to yield a colourless oil (yield: 18%).

¹H NMR (CDCl₃, 250 MHz) δ ppm: 7.64 (d, J=8.5 Hz, 2H), 7.33-7.04 (m, 8H), 6.85 (m, 3H), 6.75 (bs, 1H), 2.69-2.37 (m, 5H), 2.35 (s, 3H), 1.98 (m, 1H), 1.81-1.40 (m, 5H).

EI MS: m/z=452 (M+1), 469 (M+18).

### Example 26

### 5-{5-[(Methylamino)sulfonyl]thien-2-yl}-2-(2-phenylethyl)pentanoic acid

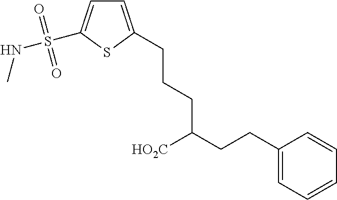

### a) N-methylthiophene-2-sulfonamide

MeNH₂ (10.3 mL, 8 M solution in EtOH, 82.12 mmol) was added to a solution of 2-thiophenesulfonyl chloride (5.0 g, 27.37 mmol) in THF (70 mL). The reaction mixture was stirred at r.t. for 2 h, poured into H₂O (100 mL) and extracted with CH₂Cl₂ (2×100 mL). The organic layer was dried over Na₂SO₄ (anhydrous), filtered and concentrated, to give 5.05 g of N-methylthiophene-2-sulfonamide, that were submitted to next step without purification (brown-coloured solid, yield: quantitative).

¹H NMR (CDCl₃, 250 MHz) δ ppm: 7.60 (m, 2H), 7.09 (dd, J=3.7 Hz, 4.0 Hz, 1H), 4.88 (bs, 1H), 2.71 (s, 3H).

### b) 5-Bromo-N-methylthiophene-2-sulfonamide

Br₂ (3 mL, 51.46 mmol) was added to a solution of N-methylthiophene-2-sulfonamide (4.56 g, 25.73 mmol) in CHCl₃ (70 mL). The reaction mixture was refluxed for 7 h, allowed to reach r.t., poured into H₂O (100 mL) and extracted with CH₂Cl₂ (100 mL). The organic layer was dried over Na₂SO₄ (anhydrous), filtered and concentrated. The crude residue was flash chromatographed on SiO₂ (15→30% EtOAc/hexanes) to furnish 1.23 g of 5-bromo-N-methylthiophene-2-sulfonamide (off-white solid, yield: 19%).

¹H NMR (CDCl₃, 250 MHz) δ ppm: 7.61 (m, 1H), 7.10 (dd, J=3.8 Hz, 4.5 Hz, 1H), 4.79 (bs, 1H), 2.73 (d, J=5.2 Hz, 3H).

### c) tert-Butyl (5-bromothien-2-yl)sulfonyl(methyl)carbamate

Boc₂O (1.17 g, 5.36 mmol) was added to a solution of 5-bromo-N-methylthiophene-2-sulfonamide (1.23 g, 4.78 mmol), DMAP (55 mg, 0.45 mmol) and DIPEA (2.3 mL, 13.67 mmol) in CH₃CN (50 mL). The reaction mixture was stirred at r.t. for 1 h, poured into H₂O (50 mL) and extracted with CH₂Cl₂ (100 mL). The organic layer was dried over Na₂SO₄ (anhydrous), filtered and concentrated. The crude residue was flash chromatographed on SiO₂ (15% EtOAc/hexanes) to furnish 1.61 g of tert-butyl (5-bromothien-2-yl)sulfonyl(methyl)carbamate (orange-coloured solid, yield: 95%).

¹H NMR (CDCl₃, 250 MHz) δ ppm: 7.49 (d, J=3.9 Hz, 1H), 7.07 (d, J=3.9 Hz, 1H), 3.28 (s, 3H), 1.48 (s, 9H).

### d) tert-Butyl[5-(3-hydroxyprop-1-ynyl)thien-2-yl]sulfonyl(methyl)carbamate

The compound was prepared from tert-butyl (5-bromothien-2-yl)sulfonyl(methyl)carbamate following the general procedure C. Flash chromatography purification on SiO₂ (20→30% EtOAc/hexanes) afforded the desired product as a yellow-coloured solid (yield: 86%).

¹H NMR (CDCl₃, 250 MHz) δ ppm: 7.57 (d, J=3.8 Hz, 1H), 7.11 (d, J=3.8 Hz, 1H), 4.52 (s, 2H), 3.29 (s, 3H), 1.83 (bs, 1H), 1.47 (s, 9H).

### e) tert-Butyl[5-(3-bromoprop-1-ynyl)thien-2-yl]sulfonyl(methyl)carbamate

The compound was prepared from tert-Butyl[5-(3-hydroxyprop-1-ynyl)thien-2-yl]sulfonyl(methyl)carbamate following the general procedure D. Flash chromatography purification on SiO₂ (15→20% EtOAc/hexanes) afforded the desired product as a white solid (yield: 92%).

¹H NMR (CDCl₃, 250 MHz) δ ppm: 7.58 (d, J=3.9 Hz, 1H), 7.16 (d, J=3.9 Hz, 1H), 4.16 (s, 2H), 3.29 (s, 3H), 1.47 (s, 9H).

### f) Methyl 5-{5-[tertbutoxycarbonylmethylamino)sulfonyl]thien-2-yl}-2-(2-phenylethyl)pent-4-ynoate

The compound was prepared from methyl 4-phenylbutanoate and tert-butyl [5-(3-bromoprop-1-ynyl)thien-2-yl]sulfonyl(methyl)carbamate following the general procedure A. Flash chromatography purification on SiO₂ (10→20% EtOAc/hexanes) afforded the desired product as a yellow-coloured oil (yield: 45%).

¹H NMR (CDCl₃, 250 MHz) δ ppm: 7.55 (d, J=3.8 Hz, 1H), 7.37-7.12 (m, 5H), 7.02 (d, J=3.8 Hz, 1H), 3.73 (s, 3H), 3.29 (s, 3H), 2.72 (m, 5H), 1.99 (m, 2H), 1.46 (s, 9H).

### g) Methyl 5-{5-[tertbutoxycarbonylmethylamino)sulfonyl]thien-2-yl}-2-(2-phenylethyl)pentanoate

The compound was prepared from methyl 5-{5-[tertbutoxycarbonylmethyl amino)sulfonyl]thien-2-yl}-2-(2-phenylethyl)pent-4-ynoate following the general procedure E. The crude residue was submitted to next step without purification (colourless oil, yield: 96%).

EI MS: m/z=496 (M+1).

### h) 5-{5-[(Methylamino)sulfonyl]thien-2-yl}-2-(2-phenylethyl)pentanoic acid

The compound was synthesized from methyl 5-{5-[tertbutoxycarbonylmethyl amino)sulfonyl]thien-2-yl}-2-(2-phenylethyl)pentanoate following the experimental procedure detailed in Method B. It was purified by flash chromatography on SiO₂ (5→10% MeOH/CH₂Cl₂) to give a yellow-coloured solid (yield: 37%).

¹H NMR (CDCl₃, 250 MHz) δ ppm: 7.42 (d, J=3.6 Hz, 1H), 7.29 (m, 2H), 7.18 (m, 3H), 6.75 (d, J=3.6 Hz, 1H), 4.75 (bs, 1H), 2.83 (m, 2H), 2.70 (bs, 3H), 2.63 (m, 1H), 2.42 (m, 2H), 1.99 (m, 1H), 1.85-1.54 (m, 5H).

EI MS: m/z=382 (M+1), 380 (M−1).

### Example 27

### 5-(3-[(Benzylamino)sulfonyl]phenyl)-2-(2-phenylethyl)pentanoic acid

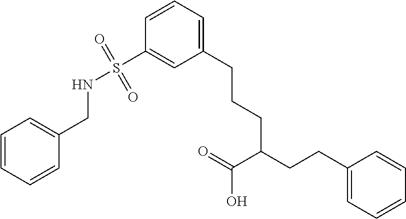

### a) N-Benzyl-3-bromobenzenesulfonamide

Benzylamine (2.6 mL, 23.48 mmol) was added to a solution of 3-bromobenzenesulfonyl chloride (2.0 g, 7.83 mmol) in THF (30 mL). The reaction mixture was stirred at r.t. for 3 h, poured into H₂O (50 mL) and extracted with CH₂Cl₂ (120 mL). The organic layer was dried over Na₂SO₄ (anhydrous), filtered and concentrated. The crude residue was flash chromatographed on SiO₂ (10→50% EtOAc/hexanes) to furnish 2.31 g of N-benzyl-3-bromobenzenesulfonamide (white solid, yield: 90%).

¹H NMR (CDCl₃, 250 MHz) δ ppm: 7.76 (m, 1H), 7.60-7.46 (m, 2H), 7.22-6.94 (m, 6H), 4.82 (t, J=5.5. Hz, 1H), 3.97 (d, J=6.0 Hz, 2H).

### b) tert-Butylbenzyl[(3-bromophenyl)sulfonyl]carbamate

Boc₂O (2.00 g, 9.20 mmol) was added to a solution of N-benzyl-3-bromobenzenesulfonamide (2.31 g, 7.08 mmol), DMAP (87 mg, 0.71 mmol) and DIPEA (3.6 mL, 21.24 mmol) in CH₃CN (30 mL). The reaction mixture was stirred at r.t. for 40 min, poured into H₂O (80 mL) and extracted with EtOAc (100 mL). The organic layer was dried over Na₂SO₄ (anhydrous), filtered and concentrated. The crude residue was flash chromatographed on SiO₂ (20→30% EtOAc/hexanes) to furnish 2.93 g of tert-butylbenzyl[(3-bromophenyl)sulfonyl]carbamate (off-white solid, yield: 97%).

¹H NMR (CDCl₃, 250 MHz) δ ppm: 7.83 (m, 1H), 7.77 (m, 1H), 7.66 (m, 1H), 7.58-7.34 (m, 6H), 5.15 (s, 2H), 1.46 (s, 9H).

### c) tert-Butylbenzyl[3-(3-hydroxyprop-1-ynyl)phenyl]sulfonylcarbamate

The compound was synthesized from tert-butylbenzyl[(3-bromophenyl)sulfonyl]carbamate following the experimental procedure detailed in Method C. It was purified by flash chromatography on SiO₂ (10→50% EtOAc/hexanes) to furnish a yellow coloured oil (yield: 72%).

¹H NMR (CDCl₃, 250 MHz) δ ppm: 7.71 (m, 1H), 7.58 (m, 2H), 7.37 (m, 6H), 5.05 (s, 2H), 4.50 (s, 2H), 1.92 (bs, 1H), 1.33 (s, 9H).

### d) tert-Butylbenzyl{[3-(3-bromoprop-1-ynyl)phenyl]sulfonyl}carbamate

The compound was synthesized from tert-butylbenzyl[3-(3-hydroxyprop-1-ynyl)phenyl]sulfonylcarbamate following the experimental procedure detailed in Method D. It was purified by flash chromatography on SiO₂ (10→20% EtOAc/hexanes) to furnish a colourless oil (yield: 85%).

¹H NMR (CDCl₃, 250 MHz) δ ppm: 7.65 (m, 1H), 7.59 (m, 2H), 7.37 (m, 6H), 5.05 (s, 2H), 4.13 (s, 2H), 1.35 (s, 9H).

### e) Methyl 5-(3-{[(tert-butyl benzyl)sulfonyl]carbamate}phenyl)-2-(2-phenylethyl)pent-4-ynoate

The compound was synthesized from tert-butylbenzyl{[3-(3-bromoprop-1-ynyl)phenyl]sulfonyl}carbamate and methyl 4-phenylbutanoate following the experimental procedure detailed in Method A. It was purified by flash chromatography on SiO₂ (5→20% EtOAc/hexanes) to furnish a colourless oil (yield: 60%).

¹H NMR (CDCl₃, 250 MHz) δ ppm: 7.68 (m, 1H), 7.51 (m, 2H), 7.41-7.23 (m, 8H), 7.19 (m, 3H), 5.03 (s, 2H), 3.74 (s, 3H), 2.66 (m, 5H), 2.62 (m, 2H), 1.31 (s, 9H).

### f) Methyl 5-(3-{[(tert-butyl benzyl)sulfonyl]carbamate}phenyl)-2-(2-phenylethyl)pentanoate

The compound was synthesized from methyl 5-(3-{[(tert-butyl benzyl)sulfonyl]carbamate}phenyl)-2-(2-phenylethyl)pent-4-ynoate following the experimental procedure detailed in Method E. It was submitted to next step without purification (colourless oil, 88%).

¹H NMR (CDCl₃, 250 MHz) δ ppm: 7.58 (m, 1H), 7.47 (m, 2H), 7.45-7.29 (m, 8H), 7.22 (m, 3H), 5.11 (s, 2H), 3.74/s, 3H), 2.74-2.36 (m, 5H), 2.00 (m, 1H), 1.85-1.44 (m, 5H), 1.33 (s, 9H).

### g) 5-(3-[(Benzylamino)sulfonyl]phenyl)-2-(2-phenylethyl)pentanoic acid

The compound was synthesized from methyl 5-(3-{[(tert-butyl benzyl)sulfonyl]carbamate}phenyl)-2-(2-phenylethyl)pentanoate following the experimental procedure detailed in Method B. It was purified by flash chromatography on SiO₂ (25→35% EtOAc/hexanes) to yield a colourless oil (yield: 62%).

¹H NMR (CDCl₃, 250 MHz) δ ppm: 7.71 (m, 2H), 7.49-7.35 (m, 2H), 7.34-7.17 (m, 10H), 4.97 (t, J=6.0 Hz, 1H), 4.17 (d, J=6.0 Hz, 2H), 2.69 (m, 4H), 2.47 (m, 1H), 2.02 (m, 1H), 1.88-1.55 (m, 5H).

EI MS: m/z=452 (M+1).

### Example 28

### 5-Phenyl-2-(2-pyridin-2-ylethyl)pentanoic acid

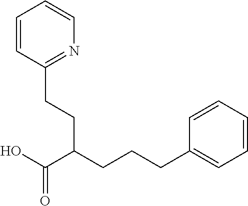

### a) Ethyl 4-pyridin-2-ylbutanoate

4-Ethoxy-4-oxobutylzinc bromide (3.3 mL, 0.5M solution in THF, 1.65 mmol) was added to a solution of 2-bromopyridine (200 mg, 1.265 mmol) and (Ph₃P)₄Pd (105 mg, 0.09 mmol) in THF (15 mL). The reaction mixture was stirred at r.t. for 3 h and poured into H₂O (50 mL). It was taken up to pH=2 with HCl (10% aqueous solution) and extracted with EtOAc (100 mL). The organic layer was dried over Na₂SO₄ (anhydrous), filtered and concentrated. The crude residue was flash chromatographed on SiO₂ (10→40% EtOAc/hexanes) to furnish 180 mg of ethyl 4-pyridin-2-ylbutanoate (yellow oil, yield: 74%).

¹H NMR (CDCl₃, 250 MHz) δ ppm: 8.50 (d, J=4.1 Hz, 1H), 7.57 (dt, J=7.6 Hz, 2.3 Hz, 1H), 7.10 (m, 2H), 4.10 (q, J=7.0 Hz, 2H), 2.81 (t, J=8.2 Hz, 2H), 2.34 (t, J=7.6 Hz, 2H), 2.06 (m, 2H), 1.23 (t, J=7.0 Hz, 3H).

### b) Ethyl 5-phenyl-2-(2-pyridin-2-ylethyl)pentanoate

The compound was synthesized from ethyl 4-pyridin-2-ylbutanoate and PhCH₂CH₂CH₂I as alkylating agent, following the experimental procedure detailed in Method A. It was purified by flash chromatography on SiO₂ (10→20% EtOAc/hexanes) to furnish a yellow coloured oil (yield: 40%).

¹H NMR (CDCl₃, 250 MHz) δ ppm: 8.52 (d, J=4.1 Hz, 1H), 7.58 (dt, J=7.6 Hz, 2.3 Hz, 1H), 7.1 (m, 3H), 7.16 (m, 4H), 4.14 (q, J=7.0 Hz, 2H), 2.76 (m, 2H), 2.59 (t, J=7.6 Hz, 2H), 2.41 (m, 1H), 2.02 (m, 2H), 1.68 (m, 4H), 1.26 (t, J=7.0 Hz, 3H).

### c) 5-Phenyl-2-(2-pyridin-2-ylethyl)pentanoic acid

The compound was synthesized from ethyl 5-phenyl-2-(2-pyridin-2-ylethyl)pentanoate following the experimental procedure detailed in Method B. It was purified by flash chromatography on SiO₂ (10→70% EtOAc/hexanes), and slurred with Et₂O to furnish a white solid (yield: 31%).

¹H NMR (CDCl₃, 250 MHz) δ ppm: 8.54 (d, J=4.1 Hz, 1H), 7.74 (t, J=7.6 Hz, 1H), 7.37-7.07 (m, 7H), 2.96 (t, J=8.2 Hz, 2H), 2.59 (q, J=8.2 Hz, 2H), 2.41 (m, 1H), 2.05 (m, 1H), 1.94-1.49 (m, 4H), 1.41 (m, 1H).

EI MS: m/z=284 (M+1).

### Example 29

### 2-[2-(3-methoxyphenyl)ethyl]-5-phenylpentanoic acid

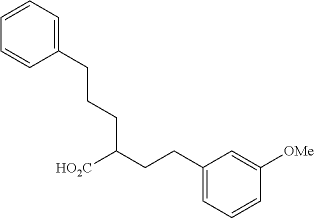

### a) Ethyl 4-(3-methoxyphenyl)butanoate

4-Ethoxy-4-oxobutylzinc bromide (13 mL, 0.5M solution in THF, 6.5 mmol) was added to a solution of 1-bromo-3-methoxybenzene (1.0 g, 5.35 mmol) and (Ph₃P)₄Pd (742 mg, 0.642 mmol) in THF (25 mL). The reaction mixture was refluxed for 2 h, allowed to reach r.t. and poured into H₂O (80 mL). It was taken up to pH=2 with HCl (10% aqueous solution) and extracted with EtOAc (90 mL). The organic layer was dried over Na₂SO₄ (anhydrous), filtered and concentrated. The crude residue was flash chromatographed on SiO₂ (0→4% EtOAc/hexanes) to furnish 677 mg of ethyl 4-(3-methoxyphenyl)butanoate (coloureless oil, yield: 57%).

EI MS: m/z=223 (M+1).

### b) Ethyl 2-[2-(3-methoxyphenyl)ethyl]-5-phenylpentanoate

The compound was synthesized from ethyl 4-(3-methoxyphenyl)butanoate and PhCH₂CH₂CH₂I, following the experimental procedure detailed in Method A. It was purified by flash chromatography on SiO₂ (0→5% EtOAc/hexanes) to furnish a yellow coloured oil (yield: 64%).

¹H NMR (CDCl₃, 250 MHz) δ ppm: 7.30 (m, 2H), 7.16 (m, 4H), 6.72 (m, 3H), 4.16 (c, J=7.1 Hz, 2H), 3.81 (s, 3H), 2.57 (m, 4H), 2.37 (m, 1H), 1.91 (m, 1H), 1.85-1.43 (m, 6H), 1.27 (t, J=7.1 Hz, 3H).

### c) 2-[2-(3-methoxyphenyl)ethyl]-5-phenylpentanoic acid

The compound was synthesized from ethyl 2-[2-(3-methoxyphenyl)ethyl]-5-phenylpentanoate following the experimental procedure detailed in Method B. It was purified by flash chromatography on SiO₂ (15→20% EtOAc/hexanes) to furnish a colourless oil (yield: 57%).

¹H NMR (CDCl₃, 250 MHz) δ ppm: 7.29-7.12 (m, 6H), 6.74 (m, 3H), 3.79 (s, 3H), 2.61 (m, 4H), 2.44 (m, 1H), 1.94 (m, 1H), 1.88-1.53 (m, 5H).

EI MS: m/z=311 (M−1).

### Example 30

### 2-[2-(3-Hydroxyphenyl)ethyl]-5-phenylpentanoic acid

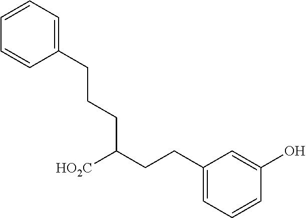

BBr₃ (2.5 mL, 1 M solution in CH₂Cl₂, 2.5 mmol) was added to a −78° C. cooled solution of 2-[2-(3-hydroxyphenyl)ethyl]-5-phenylpentanoic acid (261 mg, 0.83 mmol) in CH₂Cl₂ (15 mL). The reaction mixture was stirred at low temperature for 30 min, and allowed to reach r.t. It was stirred overnight (18 h), poured into H₂O (15 mL) and extracted with CH₂Cl₂ (50 mL). The organic layer was dried over Na₂SO₄ (anhydrous), filtered and concentrated. The crude residue was flash chromatographed on SiO₂ (15→30% EtOAc/hexanes) to furnish 85 mg of 2-[2-(3-hydroxyphenyl)ethyl]-5-phenylpentanoic acid (off-white solid, yield: 34%).

¹H NMR (CDCl₃, 250 MHz) δ ppm: 7.96 (d, J=8.5 Hz, 1H), 7.31-7.10 (m, 5H), 6.77 (m, 1H), 6.67 (m, 1H), 6.56 (bs, 1H), 2.90 (m, 2H), 2.63 (m, 2H), 2.45 (m, 1H), 2.17 (m, 1H), 2.03-1.49 (m, 5H).

EI MS: m/z=281 (M−17).

### Example 31

### 2-{2-[4-(Acetylamino)phenyl]ethyl}-5-phenylpentanoic acid

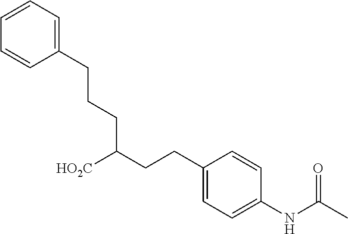

### a) Methyl 4-(4-nitrophenyl)butanoate

H₂SO₄ (0.16 mL, 2.868 mmol) was added to a solution of 4-(4-nitrophenyl)butanoic acid (2.0 g, 9.56 mmol) in MeOH (20 mL). The reaction mixture was stirred at r.t. for 16 h, poured into H₂O (50 mL) and extracted with CH₂Cl₂ (2×50 mL). The organic layer was washed with NaHCO₃ (300 mL, saturated aqueous solution), dried over Na₂SO₄ (anhydrous), filtered and concentrated. The crude residue was flash chromatographed on SiO₂ (10 EtOAc/hexanes), to furnish methyl 4-(4-nitrophenyl)butanoate (2.00 g, colourless oil, yield: 94%).

¹H NMR (CDCl₃, 250 MHz) δ ppm: 8.14 (d, J=8.8 Hz, 2H), 7.33 (d, J=8.8 Hz, 2H), 3.67 (s, 3H), 2.76 (t, J=7.9 Hz, 2H), 2.33 (t, J=7.6 Hz, 2H), 1.98 (q, J=7.7 Hz, 2H).

### b) Methyl 4-(4-aminophenyl)butanoate

A suspension of methyl 4-(4-nitrophenyl)butanoate (3.05 g, 13.67 mmol) and Pd/C (1.0 g, 10% Pd on activated C, 1.09 mmol) in MeOH (40 mL) was stirred under H₂ atmosphere (balloon) for 3 h. The reaction mixture was filtered through Celite (washing with EtOAc). Solvent was concentrated off, to furnish 2.33 g of methyl 4-(4-aminophenyl)butanoate (brown solid, yield: 88%). It was submitted to next step without purification.

¹H NMR (CDCl₃, 250 MHz) δ ppm: 6.96 (d, J=8.5 Hz, 2H), 6.63 (d, J=8.2 Hz, 2H), 3.66 (s, 3H), 2.55 (t, J=7.6 Hz, 2H), 2.31 (t, J=7.7 Hz, 2H), 1.90 (q, J=7.6 Hz, 2H).

### c) Methyl 4-[4-(N,N-dibenzylamino)phenyl]butanoate

BnBr (4.33 mL, 36.17 mmol) was added to a suspension of K₂CO₃ (5.0 g, 36.18 mmol) and methyl 4-(4-aminophenyl)butanoate (2.33 g, 12.056 mmol) in CH₃CN (60 mL). The reaction mixture was warmed up to reflux and allowed to react for 12 h. It was poured into H₂O (100 mL) and extracted with EtOAc (2×100 mL). The organic layer was dried over Na₂SO₄ (anhydrous), filtered and concentrated. The crude residue was flash chromatographed on SiO₂ (5 10% EtOAc/hexanes) to furnish 3.61 g of methyl 4-[4-(N,N-dibenzylamino)phenyl]butanoate (brown solid, yield: 80%).

¹H NMR (CDCl₃, 250 MHz) δ ppm: 7.25 (m, 10H), 6.94 (d, J=8.5 Hz, 2H), 6.62 (d, J=8.5 Hz, 2H), 4.59 (2, 4H), 3.61 (s, 3H), 2.49 (t, J=7.4 Hz, 2H), 2.28 (t, J=7.4 Hz, 2H), 1.86 (q, J=7.6 Hz, 2H).

### d) Methyl 2-{2-[4-(N,N-dibenzylamino)phenyl]ethyl}-5-phenylpentanoate

The compound was synthesized from methyl 4-[4-(N,N-dibenzylamino)phenyl]butanoate and PhCH₂CH₂CH₂I as alkylating agent, following the experimental procedure detailed in Method A. It was purified by flash chromatography on SiO₂ (5% EtOAc/hexanes) to furnish a yellow coloured oil (yield: 34%).

¹H NMR (CDCl₃, 250 MHz) δ ppm: 7.07 (m, 15H), 6.83 (d, J=8.5 Hz, 2H), 6.53 (d, J=8.5 Hz, 2H), 4.50 (s, 4H), 3.54 (s, 3H), 2.52-2.20 (m, 5H), 1.61-1.39 (m, 6H).

### e) Methyl 2-[2-(4-aminophenyl)ethyl]-5-phenylpentanoate

A suspension of methyl 2-{2-[4-(N,N-dibenzylamino)phenyl]ethyl}-5-phenylpentanoate (1.60 g, 3.254 mmol) and Pd/C (380 mg, 10% Pd on activated C, 0.36 mmol) in MeOH (30 mL) was stirred under H₂ atmosphere (balloon) for 4 h. The reaction mixture was filtered through Celite (washing with EtOAc). Solvent was concentrated off, to furnish 0.927 g of methyl 2-[2-(4-aminophenyl)ethyl]-5-phenylpentanoate (red oil, yield: 92%). It was submitted to next step without purification.

¹H NMR (CDCl₃, 250 MHz) δ ppm: 7.12 (m, 2H), 6.95 (m, 3H), 6.75 (d, J=8.3 Hz, 2H), 6.43 (d, J=8.3 Hz, 2H), 3.47 (s, 3H), 2.43-2.12 (m, 5H), 1.79-1.25 (m, 6H).

### f) Methyl 2-[2-(4-(acetylamino)phenyl)ethyl]-5-phenylpentanoate

Ac₂O (0.37 mL, 3.86 mmol) was added to a solution of methyl 2-[2-(4-aminophenyl)ethyl]-5-phenylpentanoate (926 mg, 2.97 mmol) and Et₃N (1.25 mL, 8.95 mmol) in CH₂Cl₂ (30 mL). The reaction mixture was stirred at r.t. for 4 h, poured into H₂O (100 mL), taken up to pH=2 and extracted with EtOAc (2×150 mL). The organic layer was dried over Na₂SO₄ (anhydrous), filtered and concentrated. The crude residue was flash chromatographed on SiO₂ (10→40% EtOAc/hexanes) to furnish 925 mg of methyl 2-[2-(4-(acetylamino)phenyl)ethyl]-5-phenylpentanoate (red coloured oil, yield: 88%).

¹H NMR (CDCl₃, 250 MHz) δ ppm: 7.32 (m, 2H), 7.24-6.98 (m, 7H), 3.61 (s, 3H), 2.58-2.17 (m, 5H), 2.09 (s, 3H), 1.93-1.34 (m, 6H).

### g) 2-{2-[4-(Acetylamino)phenyl]ethyl}-5-phenylpentanoic acid

The compound was synthesized from methyl 2-[2-(4-(acetylamino)phenyl)ethyl]-5-phenylpentanoate following the experimental procedure detailed in Method B. It was purified by flash chromatography on SiO₂ (10→50% EtOAc/hexanes) to furnish a yellow coloured oil (yield: 35%).

¹H NMR (CDCl₃, 250 MHz) δ ppm: 7.25 (m, 2H), 7.13 (m, 2H), 7.00 (m, 6H), 2.59 (m, 4H), 2.40 (m, 1H), 2.16 (s, 3H), 1.80 (m, 1H), 1.52 (m, 5H).

EI MS: m/z=340 (M+1), 357 (M+18).

### Example 32

### 2-[2-(4-Aminophenyl)ethyl]-5-phenylpentanoic acid

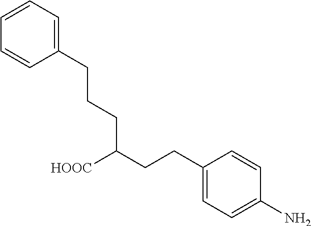

The compound was synthesized from methyl 2-[2-(4-aminophenyl)ethyl]-5-phenylpentanoate following the experimental procedure detailed in Method B. The crude residue was slurred with hexanes to furnish a white solid (yield: 63%).

¹H NMR (CDCl₃, 250 MHz) δ ppm: 7.33-7.09 (m, 5H), 6.92 (d, J=8.2, 2H). 6.70 (d, J=8.2, 2H), 2.64-2.19 (m, 5H), 1.83 (m, 1H), 1.90-1.45 (m, 5H).

EI MS: m/z=298 (M+1).

### Example 33

### 2-[2-(4-(Benzylamino)phenyl)ethyl]-5-phenylpentanoic acid

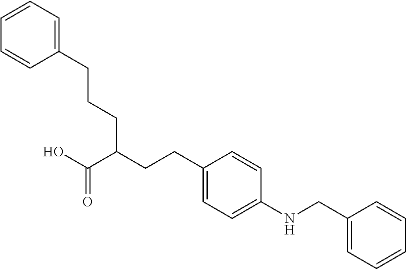

### a) Methyl 2-[2-(4-(benzylamino)phenyl)ethyl]-5-phenylpentanoate

PhCHO (390 mg, 3.71 mmol) was added to a solution of methyl 2-[2-(4-aminophenyl)ethyl]-5-phenylpentanoate (1.10 g, 3.532 mmol) in ClCH₂CH₂Cl (50 mL). The reaction mixture was stirred at r.t. for 15 min, and NaBH(OAc)₃ (1.50 g, 7.06 mmol) was added in portions. The mixture was allowed to react for 45 min, and poured into H₂O (100 mL). It was extracted with CH₂Cl₂, and the organic layer was dried over Na₂SO₄ (anhydrous), filtered and concentrated. The crude residue was flash chromatographed on SiO₂ (0→10% EtOAc/hexanes) to furnish 833 mg of methyl 2-[2-(4-(benzylamino)phenyl)ethyl]-5-phenylpentanoate (colourless oil, yield: 59%).

¹H NMR (CDCl₃, 250 MHz) δ ppm: 7.41-7.10 (m, 10H), 6.95 (d, J=8.0 Hz, 2H), 6.56 (d, J=8.0 Hz, 2H), 4.30 (s, 2H), 3.94 (bs, 1H), 3.67 (s, 3H), 2.58 (m, 2H), 2.42 (m, 3H), 1.86 (m, 1H), 1.59 (m, 6H).

### b) 2-[2-(4-(Benzylamino)phenyl)ethyl]-5-phenylpentanoic acid

The compound was synthesized from methyl 2-[2-(4-(benzylamino)phenyl)ethyl]-5-phenylpentanoate following the experimental procedure detailed in Method B. It was purified by flash chromatography on SiO₂ (5 20% EtOAc/hexanes) to furnish a yellow-coloured oil (yield: 72%).

¹H NMR (CDCl₃, 250 MHz) δ ppm: 7.51-7.19 (m, 10H), 7.06 (d, J=8.2 Hz, 2H), 6.66 (d, J=8.2 Hz, 2H), 4.37 (s, 2H), 2.75-2.43 (m, 5H), 2.05 (m, 1H), 1.88-1.58 (m, 5H).

EI MS: m/z=388 (M+1).

### Example 34

### 5-Phenyl-2-(3-phenylpropyl)pentanoic acid

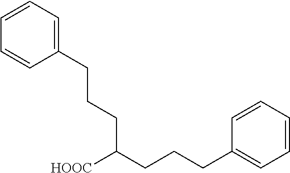

### a) Methyl 5-phenylpentanoate

H₂SO₄ (1.7 mL, 31.89 mmol) was added to a solution of 5-phenylpentanoic acid (15.0 g, 84.161 mmol) in MeOH (80 mL). The reaction mixture was stirred at r.t. for 3 h, poured into H₂O (400 mL) and extracted with CH₂Cl₂ (2×300 mL). The organic layer was washed with NaHCO₃ (300 mL, saturated aqueous solution), dried over Na₂SO₄ (anhydrous), filtered and concentrated, to furnish methyl 5-phenylpentanoate (16.30 g, colourless oil, yield: 100%). The crude residue was submitted to next step without further purification.

¹H NMR (CDCl₃, 250 MHz) δ ppm: 7.45 (m, 2H), 7.35 (m, 3H), 3.84 (s, 3H), 2.81 (t, J=7.2 Hz, 2H), 2.52 (t, J=7.2 Hz, 2H), 1.84 (m, 4H).

### b) Methyl 5-phenyl-2-(3-phenylpropyl)pentanoate

The compound was prepared from methyl 5-phenylpentanoate following the general procedure A, by using PhCH₂CH₂CH₂I as alkylating reagent. Flash chromatography purification afforded the compound as a colourless oil (yield: 16%).

¹H NMR (CDCl₃, 250 MHz) δ ppm: 7.25 (m, 10H), 7.17 (m, 5H), 3.67 (s, 3H), 2.60 (m, 3H), 2.32 (m, 2H), 1.71-1.51 (m, 8H).

### c) 5-Phenyl-2-(3-phenylpropyl)pentanoic acid

It was prepared from methyl 5-phenyl-2-(3-phenylpropyl)pentanoate following the general procedure B, to furnish the compound as a colourless oil after flash chromatography purification (yield: 15%).

¹H NMR (CDCl₃, 250 MHz) δ ppm: 7.31 (m, 5H), 7.22 (m, 5H), 2.65 (m, 3H), 2.45 (m, 1H), 1.80-1.51 (m, 9H).

EI MS: m/z=295 (M−1).

### Example 35

### 5-Phenyl-2-propylpentanoic acid

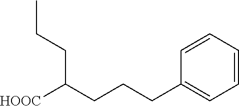

### a) Methyl 5-phenyl-2-propylpentanoate

The compound was prepared from methyl 5-phenylpentanoate following the general procedure A, by using Prl as alkylating reagent. Flash chromatography purification afforded the compound as a yellow coloured oil (yield: 55%).

¹H NMR (CDCl₃, 250 MHz) δ ppm: 7.31 (m, 2H), 7.20 (m, 3H), 3.72 (s, 3H), 2.65 (t, J=7.4 Hz, 2H), 2.42 (m, 1H), 1.73-1.55 (m, 4H), 1.50-1.26 (m, 4H), 0.94 (t, J=7.4 Hz, 3H).

### b) 5-Phenyl-2-propylpentanoic acid

It was prepared from methyl 5-phenyl-2-propylpentanoate following the general procedure B, to furnish the compound as a colourless oil after flash chromatography purification (yield: 62%).

¹H NMR (CDCl₃, 250 MHz) δ ppm: 7.29 (m, 2H), 7.20 (m, 3H), 2.66 (t, J=7.4 Hz, 2H), 2.42 (m, 1H), 1.77-1.55 (m, 4H), 1.52-1.27 (m, 4H), 0.93 (t, J=7.4 Hz, 3H).

EI MS: m/z=219 (M−1).

### Example 36

### 5-(3-[(4-Methylanilinosulfonyl]phenyl)-2-(2-phenylethyl)pentanoic acid

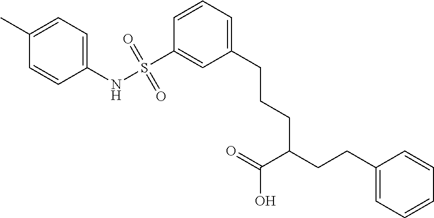

### a) 3-Bromo-N-(4-methylphenyl)benzenesulfonamide

p-Toluidine (1.68 g, 15.655 mmol) was added to a solution of 3-bromobenzenesulfonyl chloride (2.0 g, 7.827 mmol) and Et₃N (2.19 mL, 15.655 mmol) in THF (60 mL). The reaction mixture was allowed to react for 1 h and poured into H₂O (120 mL). It was taken up to pH=1 with HCl (15% aqueous solution) and extracted with EtOAc (2×100 mL). The organic layer was dried over Na₂SO₄ (anhydrous), filtered and concentrated, to furnish the product as a waxy solid. The crude residue was submitted to next step without purification.

¹H NMR (CDCl₃, 250 MHz) δ ppm: 7.91 (t, J=1.9 Hz, 1H), 7.65 (dd, J=6.3 Hz, 1.6 Hz, 2H), 7.30 (t, J=7.9 Hz, 1H), 7.02 (m, 4H), 6.83 (bs, 1H), 2.28 (s, 3H).

### b) tert-Butyl (3-bromophenyl)sulfonyl(4-methylphenyl)carbamate

Boc₂O (2.22 g, 10.175 mmol) was added to a solution of 3-bromo-N-(4-methylphenyl)benzenesulfonamide (7.827 mmol), DMAP (95 mg, 0.782 mmol) and DIPEA (4.0 mL, 23.48 mmol) in CH₃CN (60 mL). The reaction mixture was stirred at r.t. for 2 h, poured into H₂O (200 mL) and extracted with EtOAc (200 mL). The organic layer was dried over Na₂SO₄ (anhydrous), filtered and concentrated. The crude residue was flash chromatographed on SiO₂ (5 10% EtOAc/hexanes), to afford 2.90 g of tert-butyl (3-bromophenyl)sulfonyl(4-methylphenyl)carbamate (white solid, yield: 87%).

¹H NMR (CDCl₃, 250 MHz) δ ppm: 8.15 (t, J=1.9 Hz, 1H), 7.90 (m, 1H), 7.78 (m, 1H), 7.43 (t, J=7.9 Hz, 1H), 7.24 (d, J=7.4 Hz, 2H), 7.13 (d, J=8.5 Hz, 2H), 2.40 (s, 3H), 1.37 (s, 9H).

### c) tert-Butyl[3-(3-hydroxyprop-1-ynyl)phenyl]sulfonyl(4-methylphenyl) carbamate

The compound was synthesized from tert-butyl (3-bromophenyl)sulfonyl(4-methylphenyl)carbamate following the experimental procedure detailed in Method C. It was purified by flash chromatography on SiO₂ (10→40% EtOAc/hexanes) to furnish an orange coloured solid (yield: 89%).

¹H NMR (CDCl₃, 250 MHz) δ ppm: 8.05 (s, 1H), 7.92 (d, J=7.9 Hz, 1H), 7.66 (d, J=7.6 Hz, 1H), 7.48 (t, J=7.9 Hz, 1H), 7.24 (d, J=8.2 Hz, 2H), 7.15 (d, J=8.2 Hz, 2H), 4.47 (s, 2H), 2.91 (bs, 1H), 2.38 (s, 3H), 1.34 (s, 9H).

### d) tert-Butyl[3-(3-bromoprop-1-ynyl)phenyl]sulfonyl(4-methylphenyl)carbamate

The compound was synthesized from tert-butyl[3-(3-hydroxyprop-1-ynyl)phenyl]sulfonyl(phenyl)carbamate following the experimental procedure detailed in Method D. It was purified by flash chromatography on SiO₂ (5→20% EtOAc/hexanes) to furnish a white solid (yield: 85%).

¹H NMR (CDCl₃, 250 MHz) δ ppm: 8.07 (t, J=1.3 Hz, 1H), 7.93 (m, 1H), 7.69 (m, 1H), 7.51 (t, J=7.9 Hz, 1H), 7.25 (d, J=7.4 Hz, 2H), 7.13 (d, J=8.5 Hz, 2H), 4.15 (s, 2H), 2.40 (s, 3H), 1.36 (s, 9H).

### e) Methyl 5-[3-(tert-butoxycarbonyl-4-methylanilinosulfonyl)phenyl]-2-(2-phenylethyl)pent-4-ynoate

The compound was synthesized from tert-butyl[3-(3-bromoprop-1-ynyl)phenyl]sulfonyl(phenyl)carbamate and methyl 4-phenylbutanoate following the experimental procedure detailed in Method A. It was purified by flash chromatography on SiO₂ (5-30% EtOAc/hexanes) to furnish a white solid (yield: 72%).

¹H NMR (CDCl₃, 250 MHz) δ ppm: 7.99 (m, 1H), 7.88 (m, 1H), 7.61 (m, 1H), 7.46 (t, J=7.4 Hz, 1H), 7.35-7.09 (m, 9H), 3.74 (s, 3H), 2.83-2.57 (m, 5H), 2.39 (s, 3H), 2.25-1.94 (m, 2H), 1.34 (s, 9H).

### f) Methyl 5-[3-(tert-butoxycarbonyl-4-methylanilinosulfonyl)phenyl]-2-(2-phenylethyl)pentanoate

The compound was synthesized from methyl 5-[3-(tert-butoxycarbonyl anilinosulfonyl)phenyl]-2-(2-phenylethyl)pent-4-ynoate following the experimental procedure detailed in Method E to furnish a waxy solid (yield: 94%). The crude residue was submitted to next step without purification.

¹H NMR (CDCl₃, 250 MHz) δ ppm: 7.77 (m, 2H), 7.43 (m, 2H), 7.34-7.19 (m, 5H), 7.14 (t, J=7.6 Hz, 4H), 3.69 (s, 3H), 2.74-2.51 (m, 4H), 2.39 (s, 3H), 2.07-1.87 (m, 1H), 1.83-1.46 (m, 6H), 1.33 (s, 9H).

### g) 5-(3-[(4-Methylanilinosulfonyl]phenyl)-2-(2-phenylethyl)pentanoic acid

The compound was synthesized from methyl 5-[3-(tert-butoxycarbonyl anilinosulfonyl)phenyl]-2-(2-phenylethyl)pentanoate following the experimental procedure detailed in Method B. It was purified by flash chromatography on SiO₂ (10 30% EtOAc/hexanes) to yield a white solid (yield: 56%).

¹H NMR (CDCl₃, 250 MHz) δ ppm: 7.59-7.47 (m, 2H), 7.27 (m, 5H), 7.18 (t, J=8.2 Hz, 2H), 7.01 (d, J=8.5 Hz, 2H), 6.93 (d, J=8.5 Hz, 2H), 6.76 (bs, 1H), 2.79-2.52 (m, 4H), 2.51-2.34 (m, 1H), 2.24 (s, 3H), 2.11-1.87 (m, 1H), 1.85-1.42 (m, 5H).

EI MS: m/z=452 (M+1).

### Example 37

### Sodium 5-(3-[(4-methylanilinosulfonyl]phenyl)-2-(2-phenylethyl)pentanoate

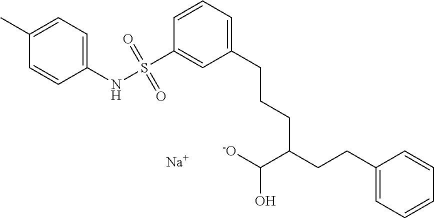

NaH (16 mg, 60% mineral oil suspension, 0.40 mmol) was added to a solution of 5-(3-[(4-methylanilinosulfonyl]phenyl)-2-(2-phenylethyl)pentanoic acid (Example 36) (180 mg, 0.398 mmol) in THF (10 mL). The reaction mixture was stirred at r.t. for 15 min, and solvent was concentrated off. The crude residue was slurred with Et₂O (5 mL) and hexanes (5 mL), to give 120 mg of sodium 5-(3-[(4-methylanilinosulfonyl]phenyl)-2-(2-phenylethyl)pentanoate (white solid, yield: 64%).

¹H NMR (MeOD, 250 MHz) δ ppm: 7.50 (m, 2H), 7.34 (m, 2H), 7.17 (m, 5H), 6.93 (m, 4H), 2.66 (m, 4H), 2.27 (m, 1H), 2.20 (s, 3H), 1.81 (m, 1H), 1.59 (m, 4H).

EI MS: m/z=452 (M−Na+1).

### Example 38

### 5-[3-(4-Methylanilinosulfonyl)phenyl]-2-(2-phenylethyl)pent-4-ynoic acid

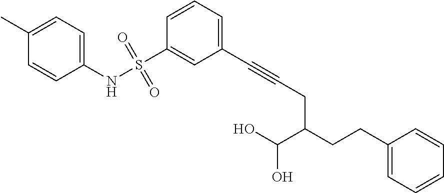

The compound was synthesized from methyl 5-[3-(tert-butoxycarbonyl-4-methylanilinosulfonyl)phenyl]-2-(2-phenylethyl)pent-4-ynoate following the experimental procedure detailed in Method B. It was purified by flash chromatography on SiO₂ (10→50% EtOAc/hexanes) to yield a colourless oil (yield: 40%).

¹H NMR (CDCl₃, 250 MHz) δ ppm: 7.82 (s, 1H), 7.58 (d, J=7.9, 1H), 7.48 (d, J=6.7 Hz, 1H), 7.35-7.15 (m, 6H), 7.04-6.88 (m, 5H), 2.78-2.69 (m, 5H), 2.26 (m, 3H), 2.17-2.01 (m, 2H).

EI MS: m/z=448 (M+1), 465 (M+18).

### Example 39

### 5-(3-[(Anilinosulfonyl]phenyl)-2-(2-phenylethyl)pentanoic acid

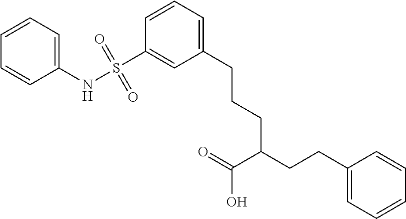

### a) 3-Bromo-N-phenylbenzenesulfonamide

Aniline (1.42 g, 15.655 mmol) was added to a solution of 3-bromobenzenesulfonyl chloride (2.0 g, 7.827 mmol) and Et₃N (2.19 mL, 15.655 mmol) in THF (60 mL). The reaction mixture was allowed to react for 1 h and poured into H₂O (120 mL). It was taken up to pH=1 with HCl (15% aqueous solution) and extracted with EtOAc (2×100 mL). The organic layer was dried over Na₂SO₄ (anhydrous), filtered and concentrated, to furnish the product as a waxy solid. The crude residue was submitted to next step without purification.

### b) tert-Butyl (3-bromophenyl)sulfonyl(phenyl)carbamate

Boc₂O (2.22 g, 10.175 mmol) was added to a solution of 3-bromo-N-phenylbenzenesulfonamide (7.827 mmol), DMAP (95 mg, 0.782 mmol) and DIPEA (4.0 mL, 23.48 mmol) in CH₃CN (60 mL). The reaction mixture was stirred at r.t. for 2 h, poured into H₂O (200 mL) and extracted with EtOAc (200 mL). The organic layer was dried over Na₂SO₄ (anhydrous), filtered and concentrated. The crude residue was flash chromatographed on SiO₂ (5→10% EtOAc/hexanes), to afford 2.55 g of tert-butyl (3-bromophenyl)sulfonyl(phenyl)carbamate (white solid, yield: 79%).

¹H NMR (CDCl₃, 250 MHz) δ ppm: 8.21 (t, J=1.6 Hz, 1H), 7.96 (m, 1H), 7.84 (m, 1H), 7.49 (m, 4H), 7.30 (m, 2H), 1.43 (s, 9H).

### c) tert-Butyl[3-(3-hydroxyprop-1-ynyl)phenyl]sulfonyl(phenyl)carbamate

The compound was synthesized from tert-butyl (3-bromophenyl)sulfonyl(phenyl)carbamate following the experimental procedure detailed in Method C. It was purified by flash chromatography on SiO₂ (10→50% EtOAc/hexanes) to furnish a yellow coloured oil (yield: 75%).

¹H NMR (CDCl₃, 250 MHz) δ ppm: 7.90 (m, 1H), 7.79 (m, 1H), 7.53 (m, 1H), 7.41-7.24 (m, 4H), 7.10 (m, 2H), 4.38 (s, 2H), 1.43 (bs, 1H), 1.19 (s, 9H).

### d) tert-Butyl[3-(3-bromoprop-1-ynyl)phenyl]sulfonyl(phenyl)carbamate

The compound was synthesized from tert-butyl[3-(3-hydroxyprop-1-ynyl)phenyl]sulfonyl(phenyl)carbamate following the experimental procedure detailed in Method D. It was purified by flash chromatography on SiO₂ (0→5% EtOAc/hexanes) to furnish a waxy solid (yield: 88%).

¹H NMR (CDCl₃, 250 MHz) δ ppm: 8.13 (s, 1H), 8.00 (m, 1H), 7.76 (m, 1H), 7.67-7.44 (m, 4H), 7.32 (m, 2H), 4.23 (s, 2H), 1.41 (s, 9H).

### e) Methyl 5-[3-(tert-butoxycarbonylanilinosulfonyl)phenyl]-2-(2-phenylethyl)pent-4-ynoate

The compound was synthesized from tert-butyl[3-(3-bromoprop-1-ynyl)phenyl]sulfonyl(phenyl)carbamate and methyl 4-phenylbutanoate following the experimental procedure detailed in Method A. It was purified by flash chromatography on SiO₂ (10→20% EtOAc/hexanes) to furnish a colourless oil (yield: 57%).

¹H NMR (CDCl₃, 250 MHz) δ ppm: 7.98 (m, 1H), 7.88 (m, 1H), 7.62 (m, 1H), 7.51-7.40 (m, 3H), 7.33-7.15 (m, 6H), 3.73 (s, 3H), 2.70 (m, 5H), 2.11 (m, 2H), 1.34 (s, 9H).

### f) Methyl 5-[3-(tert-butoxycarbonylanilinosulfonyl)phenyl]-2-(2-phenylethyl)pentanoate

The compound was synthesized from methyl 5-[3-(tert-butoxycarbonylanilinosulfonyl)phenyl]-2-(2-phenylethyl)pent-4-ynoate following the experimental procedure detailed in Method E to furnish a yellow oil (yield: 92%). The crude residue was submitted to next step without purification.

¹H NMR (CDCl₃, 250 MHz) δ ppm: 7.79 (m, 2H), 7.42 (m, 5H), 7.30-7.12 (m, 7H), 3.68 (s, 3H), 2.73-2.50 (m, 4H), 2.42 (m, 1H), 1.95 (m, 1H), 1.79-1.49 (m, 5H), 1.32 (s, 9H).

### g) 5-(3-[(Anilinosulfonyl]phenyl)-2-(2-phenylethyl)pentanoic acid

The compound was synthesized from methyl 5-[3-(tert-butoxycarbonylanilinosulfonyl)phenyl]-2-(2-phenylethyl)pentanoate following the experimental procedure detailed in Method B. It was purified by flash chromatography on SiO₂ (5→20% EtOAc/hexanes) to yield a yellow coloured oil (yield: 45%).

¹H NMR (CDCl₃, 250 MHz) δ ppm: 7.51 (m, 2H), 7.31-7.22 (m, 5H), 7.17 (m, 4H), 7.04 (m, 3H), 6.75 (bs, 1H), 2.58 (m, 4H), 2.37 (m, 1H), 1.96 (m, 1H), 1.81-1.41 (m, 5H).

EI MS: m/z=438 (M+1), 455 (M+18).

### Example 40

### 5-(4-[Anilinosulfonyl]phenyl)-2-(2-phenylethyl)pentanoic acid

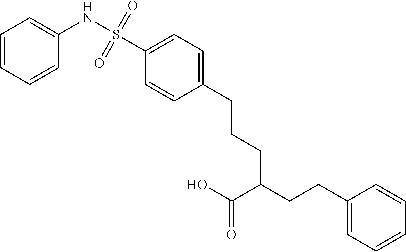

### a) 4-Bromo-N-phenylbenzenesulfonamide

Aniline (1.837 g, 19.725 mmol) was added to a solution of 4-bromobenzenesulfonyl chloride (2.5 g, 9.784 mmol) and DIPEA (3 mL, 17.524 mmol) in THF (50 mL). The reaction mixture was allowed to react for 30 min and poured into H₂O (120 mL). It was taken up to pH=1 with HCl (15% aqueous solution) and extracted with EtOAc (2×100 mL). The organic layer was dried over Na₂SO₄ (anhydrous), filtered and concentrated, to furnish the product as a yellow coloured oil. The crude residue was submitted to next step without purification.

### b) tert-Butyl (4-bromophenyl)sulfonyl(phenyl)carbamate

Boc₂O (2.80 g, 12.829 mmol) was added to a solution of 4-bromo-N-phenylbenzenesulfonamide (9.784 mmol), DMAP (150 mg, 1.227 mmol) and DIPEA (5.0 mL, 29.207 mmol) in CH₃CN (80 mL). The reaction mixture was stirred at r.t. for 2 h, poured into H₂O (200 mL) and extracted with EtOAc (200 mL). The organic layer was dried over Na₂SO₄ (anhydrous), filtered and concentrated. The crude residue was flash chromatographed on SiO₂ (5→10% EtOAc/hexanes), to afford 3.86 g of tert-butyl (4-bromophenyl)sulfonyl(phenyl)carbamate (white solid, yield: 79%).

EI MS: m/z=413 (M+1).

### c) tert-Butyl[4-(3-hydroxyprop-1-ynyl)phenyl]sulfonyl(phenyl)carbamate

The compound was synthesized from tert-butyl (4-bromophenyl)sulfonyl(phenyl)carbamate following the experimental procedure detailed in Method C. It was purified by flash chromatography on SiO₂ (20→80% EtOAc/hexanes) to furnish an orange coloured solid (yield: 73%).

¹H NMR (CDCl₃, 250 MHz) δ ppm: 7.99 (d, J=8.6 Hz, 2H), 7.64 (d, J=8.6 Hz, 2H), 7.48 (m, 3H), 7.30 (m, 2H), 4.58 (s, 2H), 1.39 (s, 9H).

### d) tert-Butyl[4-(3-bromoprop-1-ynyl)phenyl]sulfonyl(phenyl)carbamate

The compound was synthesized from tert-butyl[4-(3-hydroxyprop-1-ynyl)phenyl]sulfonyl(phenyl)carbamate following the experimental procedure detailed in Method D. It was purified by flash chromatography on SiO₂ (10→40% EtOAc/hexanes) to furnish a yellow coloured solid (yield: 99%).

¹H NMR (CDCl₃, 250 MHz) δ ppm: 7.81 (d, J=8.6 Hz, 2H), 7.47 (d, J=8.6 Hz, 2H), 7.28 (m, 3H), 7.09 (m, 2H), 4.04 (s, 2H), 1.21 (s, 9H).

### e) Methyl 5-[4-(tert-butoxycarbonylanilinosulfonyl)phenyl]-2-(2-phenylethyl)pent-4-ynoate

The compound was synthesized from tert-butyl[4-(3-bromoprop-1-ynyl)phenyl]sulfonyl(phenyl)carbamate and methyl 4-phenylbutanoate following the experimental procedure detailed in Method A. It was purified by flash chromatography on SiO₂ (5→15% EtOAc/hexanes) to furnish a yellow coloured oil (yield: 80%).

¹H NMR (CDCl₃, 250 MHz) δ ppm: 7.73 (d, J=8.3 Hz, 2H), 7.34 (d, J=8.3 Hz, 2H), 7.24 (m, 3H), 7.15-6.95 (m, 7H), 3.58 (s, 3H), 2.52 (m, 5H), 1.94 (m, 2H), 1.18 (s, 9H).

### f) Methyl 5-[4-(tert-butoxycarbonylanilinosulfonyl)phenyl]-2-(2-phenylethyl)pentanoate

The compound was synthesized from methyl 5-[4-(tert-butoxycarbonyl anilinosulfonyl)phenyl]-2-(2-phenylethyl)pent-4-ynoate following the experimental procedure detailed in Method E. It was purified by flash chromatography on SiO₂ (4→12% EtOAc/hexanes) to furnish a colourless oil (yield: 73%).

¹H NMR (CDCl₃, 250 MHz) δ ppm: 7.87 (d, J=8.3 Hz, 2H), 7.42 (m, 3H), 7.37-7.11 (m, 9H), 3.70 (s, 3H), 2.69 (m, 2H), 2.57 (m, 2H), 2.41 (m, 1H), 1.92 (m, 1H), 1.81-1.48 (m, 5H), 1.35 (s, 9H).

### g) 5-(4-[Anilinosulfonyl]phenyl)-2-(2-phenylethyl)pentanoic acid

The compound was synthesized from methyl 5-[4-(tert-butoxycarbonyl anilinosulfonyl)phenyl]-2-(2-phenylethyl)pentanoate following the experimental procedure detailed in Method B. It was purified by flash chromatography on SiO₂ (10→50% EtOAc/hexanes) to yield a white solid (yield: 53%).

¹H NMR (CDCl₃, 250 MHz) δ ppm: 7.93 (d, J=8.5 Hz, 2H), 7.68 (bs, 1H), 7.56-7.24 (m, 12H), 2.83 (m, 4H), 2.65 (m, 1H), 2.24 (m, 1H), 2.09-1.69 (m, 5H).

EI MS: m/z=438 (M+1), 455 (M+18).

### Example 41

### 5-(3-[(Methylamino)sulfonyl]phenyl)-2-(2-phenylethyl)pentanoic acid

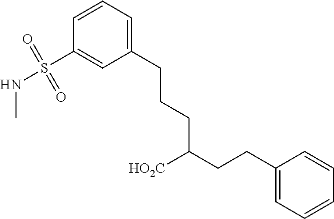

### a) 3-Bromo-N-methylbenzenesulfonamide

MeNH₂ (6 mL, 8 M solution in EtOH, 48 mmol) was added to a solution of 3-bromobenzenesulfonyl chloride (3.50 g, 13.698 mmol) in THF (60 mL). The reaction mixture was allowed to react for 5 min and poured into H₂O (200 mL) and extracted with CH₂Cl₂ (2×100 mL). The organic layer was dried over Na₂SO₄ (anhydrous), filtered and concentrated, to furnish the product as a yellow coloured solid. The crude residue was submitted to next step without purification.

¹H NMR (CDCl₃, 250 MHz) δ ppm: 8.01 (t, J=1.6 Hz, 1H), 7.82-7.68 (m, 2H), 7.41 (t, J=8.0 Hz, 1H), 4.70 (bs, 1H), 2.68 (d, J=4.9 Hz, 3H).

### b) tert-Butyl (3-bromophenyl)sulfonyl(methyl)carbamate

Boc₂O (3.41 g, 15.624 mmol) was added to a solution of 3-bromo-N-methylbenzenesulfonamide (13.698 mmol), DMAP (166 mg, 1.358 mmol) and DIPEA (7.0 mL, 40.89 mmol) in CH₃CN (70 mL). The reaction mixture was stirred at r.t. for 15 min, poured into H₂O (200 mL) and extracted with EtOAc (200 mL). The organic layer was dried over Na₂SO₄ (anhydrous), filtered and concentrated. The crude residue was flash chromatographed on SiO₂ (2→10% EtOAc/hexanes), to afford 4.72 g of tert-butyl (3-bromophenyl)sulfonyl(methyl)carbamate (white solid, yield: 89%).

¹H NMR (CDCl₃, 250 MHz) δ ppm: 8.04 (t, J=1.9 Hz, 1H), 7.89-7.71 (m, 2H), 7.40 (t, J=8.0 Hz, 1H), 3.35 (s, 3H), 1.37 (s, 9H).

### c) tert-Butyl[3-(3-hydroxyprop-1-ynyl)phenyl]sulfonyl(methyl)carbamate

The compound was synthesized from tert-butyl (3-bromophenyl)sulfonyl (methyl)carbamate following the experimental procedure detailed in Method C. It was purified by flash chromatography on SiO₂ (20 40% EtOAc/hexanes) to furnish a yellow coloured oil (yield: 40%).

¹H NMR (CDCl₃, 250 MHz) δ ppm: 7.93 (t, J=1.1 Hz, 1H), 7.83 (m, 1H), 7.64 (m, 1H), 7.46 (t, J=7.6 Hz, 1H), 4.50 (s, 2H), 3.37 (s, 3H), 1.36 (s, 9H).

### d) tert-Butyl[3-(3-bromoprop-1-ynyl)phenyl]sulfonyl(methyl)carbamate

The compound was synthesized from tert-butyl[3-(3-hydroxyprop-1-ynyl)phenyl]sulfonyl(methyl)carbamate following the experimental procedure detailed in Method D. It was purified by flash chromatography on SiO₂ (5→20% EtOAc/hexanes) to furnish a colourless oil (yield: 86%).

¹H NMR (CDCl₃, 250 MHz) δ ppm: 7.95 (t, J=1.7 Hz, 1H), 7.85 (m, 1H), 7.65 (m, 1H), 7.48 (t, J=7.6 Hz, 1H), 4.14 (s, 2H), 3.36 (s, 3H), 1.36 (s, 9H).

### e) Methyl 5-[3-(tert-butoxycarbonylmethylaminosulfonyl)phenyl]-2-(2-phenylethyl)pent-4-ynoate

The compound was synthesized from tert-butyl[3-(3-bromoprop-1-ynyl)phenyl]sulfonyl(methyl)carbamate and methyl 4-phenylbutanoate following the experimental procedure detailed in Method A. It was purified by flash chromatography on SiO₂ (2→15% EtOAc/hexanes) to furnish a colourless oil (yield: 75%).

EI MS: m/z=486 (M+1).

### f) Methyl 5-[3-(tert-butoxycarbonylmethylaminosulfonyl)phenyl]-2-(2-phenylethyl)pentanoate

The compound was synthesized from methyl 5-[3-(tert-butoxycarbonyl methylaminosulfonyl)phenyl]-2-(2-phenylethyl)pent-4-ynoate following the experimental procedure detailed in Method E to furnish a colourless oil (yield: 96%). The crude residue was submitted to next step without purification.

¹H NMR (CDCl₃, 250 MHz) δ ppm: 7.61 (m, 2H), 7.36 (m, 2H), 7.20 (m, 2H), 7.11 (m, 3H), 3.64 (s, 3H), 3.31 (s, 3H), 2.67-2.42 (m, 4H), 2.35 (m, 1H), 1.88 (m, 1H), 1.72-1.39 (m, 5H), 1.26 (s, 9H).

### g) 5-(3-[(Methylamino)sulfonyl]phenyl)-2-(2-phenylethyl)pentanoic acid

The compound was synthesized from methyl 5-[3-(tert-butoxycarbonylmethylaminosulfonyl)phenyl]-2-(2-phenylethyl)pentanoate following the experimental procedure detailed in Method B. It was purified by flash chromatography on SiO₂ (10→50% EtOAc/hexanes) to yield a colourless oil (yield: 87%).

¹H NMR (CDCl₃, 250 MHz) δ ppm: 7.70 (m, 2H), 7.41 (m, 2H), 7.30 (m, 2H), 7.20 (m, 3H), 4.69 (c, J=2.7 Hz, 1H), 2.76-2.57 (m, 7H), 2.44 (m, 1H), 1.99 (m, 1H), 1.84-1.50 (m, 5H).

EI MS: m/z=376 (M+1), 393 (M+18).

### Example 42

### Methyl 5-(3-[(methylamino)sulfonyl]phenyl)-2-(2-phenylethyl)pentanoate

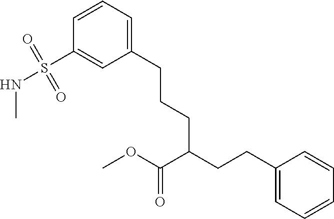

H₂SO₄ (0.05 mL) was added to a solution of 5-(3-[(methylamino)sulfonyl]phenyl)-2-(2-phenylethyl)pentanoic acid (Example 41) (350 mg, 0.932 mmol) in MeOH (25 mL). The reaction mixture was warmed up to reflux and allowed to react for 3 h. It was poured into H₂O (150 mL) and extracted with CH₂Cl₂ (150 mL). The organic layer was dried over Na₂SO₄ (anhydrous), filtered and concentrated. The crude residue was flash chromatographed on SiO₂ (10→40% EtOAc/hexanes), to afford 264 mg of methyl 5-(3-[(methylamino)sulfonyl]phenyl)-2-(2-phenylethyl)pentanoate (colourless oil, yield: 73%).

¹H NMR (CDCl₃, 250 MHz) δ ppm: 7.67 (m, 2H), 7.39 (m, 2H), 7.26 (m, 2H), 7.18 (m, 3H), 4.42 (c, J=5.4 Hz, 1H), 3.70 (s, 3H), 2.65 (d, J=5.4 Hz, 3H), 2.58 (m, 4H), 2.41 (m, 1H), 1.92 (m, 1H), 1.78-1.44 (m, 5H).

EI MS: m/z=390 (M+1), 407 (M+18).

### Example 43

### 5-(3-{[(3,4-dimethoxyphenyl)amino]sulfonyl}phenyl)-2-(2-phenylethyl)pentanoic acid

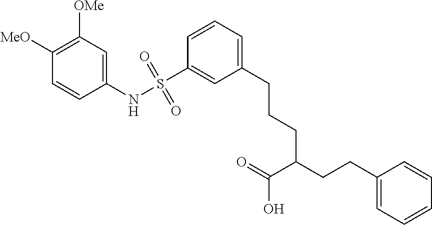

### a) 3-Bromo-N-(3,4-dimethoxyphenyl)benzenesulfonamide

3,4-Dimethoxyaniline (2.0 g, 13.056 mmol) was added to a solution of 3-bromobenzenesulfonyl chloride (1.75 g, 6.849 mmol) and Et₃N (2 mL, 14.349 mmol) in THF (40 mL). The reaction mixture was allowed to react for 30 min, poured into H₂O (200 mL) and taken to pH=2. It was extracted with EtOAc (200 mL). The organic layer was dried over Na₂SO₄ (anhydrous), filtered and concentrated, to furnish the product as a brown coloured oil. The crude residue was submitted to next step without purification.

### b) tert-Butyl (3-bromophenyl)sulfonyl(3,4-dimethoxyphenyl)carbamate

Boc₂O (1.85 g, 8.476 mmol) was added to a solution of 3-bromo-N-(3,4-dimethoxyphenyl)benzenesulfonamide (6.849 mmol), DMAP (100 mg, 0.818 mmol) and DIPEA (3.0 mL, 17.524 mmol) in CH₃CN (100 mL). The reaction mixture was stirred at r.t. for 45 min, poured into H₂O (200 mL) and extracted with EtOAc (2×120 mL). The organic layer was dried over Na₂SO₄ (anhydrous), filtered and concentrated. The crude residue was flash chromatographed on SiO₂ (10→30% EtOAc/hexanes), to afford 3.12 g of tert-butyl (3-bromophenyl)sulfonyl(3,4-dimethoxyphenyl)carbamate (white solid, yield: 96%).

¹H NMR (CDCl₃, 250 MHz) δ ppm: 8.14 (t, J=1.9 Hz, 1H), 7.92 (m, 1H), 7.79 (m, 1H), 7.44 (t, J=8.2 Hz, 1H), 6.92-6.71 (m, 3H), 3.91 (s, 3H), 3.88 (s, 3H), 1.39 (s, 9H).

### c) tert-Butyl[3-(3-hydroxyprop-1-ynyl)phenyl]sulfonyl(3,4-dimethoxyphenyl)carbamate

The compound was synthesized from tert-butyl (3-bromophenyl)sulfonyl(3,4-dimethoxyphenyl)carbamate following the experimental procedure detailed in Method C. It was purified by flash chromatography on SiO₂ (10→60% EtOAc/hexanes) to furnish a red coloured solid (yield: 89%).

¹H NMR (CDCl₃, 250 MHz) δ ppm: 8.05 (t, J=1.6 Hz, 1H), 7.94 (m, 1H), 7.69 (m, 1H), 7.51 (t, J=7.7 Hz, 1H), 6.90-6.72 (m, 3H), 4.51 (d, J=6.2 Hz, 2H), 3.91 (s, 3H), 3.88 (s, 3H), 1.88 (t, J=6.2 Hz, 1H), 1.36 (s, 9H).

### d) tert-Butyl[3-(3-bromoprop-1-ynyl)phenyl]sulfonyl(3,4-dimethoxyphenyl)carbamate

The compound was synthesized from tert-butyl[3-(3-hydroxyprop-1-ynyl)phenyl]sulfonyl(3,4-dimethoxyphenyl)carbamate following the experimental procedure detailed in Method D. It was purified by flash chromatography on SiO₂ (20→30% EtOAc/hexanes) to furnish a colourless oil (yield: 97%).

¹H NMR (CDCl₃, 250 MHz) δ ppm: 8.03 (bs, 1H), 7.92 (m, 1H), 7.66 (m, 1H), 7.49 (t, J=7.9 Hz, 1H), 6.89-6.69 (m, 3H), 4.11 (s, 2H), 3.88 (s, 3H), 3.84 (s, 3H), 1.35 (s, 9H).

### e) Methyl 5-[3-(tert-butoxycarbonyl(3,4-dimethoxyanilino)sulfonyl)phenyl]-2-(2-phenylethyl)pent-4-ynoate

The compound was synthesized from tert-butyl[3-(3-bromoprop-1-ynyl)phenyl]sulfonyl(3,4-dimethoxyphenyl)carbamate and methyl 4-phenyl butanoate following the experimental procedure detailed in Method A. It was purified by flash chromatography on SiO₂ (10→40% EtOAc/hexanes) to furnish a colourless oil (yield: 89%).

EI MS: m/z=608 (M+1).

### f) Methyl 5-[3-(tert-butoxycarbonyl(3,4-dimethoxyanilino)sulfonyl)phenyl]-2-(2-phenylethyl)pentanoate

The compound was synthesized from methyl 5-[3-(tert-butoxycarbonyl(3,4-dimethoxyanilino)sulfonyl)phenyl]-2-(2-phenylethyl)pent-4-ynoate following the experimental procedure detailed in Method E. It was purified by flash chromatography on SiO₂ (10→40% EtOAc/hexanes) to furnish a colourless oil (yield: 85%).

¹H NMR (CDCl₃, 250 MHz) δ ppm: 7.79 (m, 2H), 7.44 (m, 2H), 7.32-7.11 (m, 5H), 6.94-6.72 (m, 3H), 3.91 (s, 3H), 3.86 (s, 3H), 2.73-2.49 (m, 4H), 2.40 (m, 1H), 1.96 (m, 1H), 1.78-1.54 (m, 5H), 1.35 (s, 9H).

### g) 5-(3-{[(3,4-dimethoxyphenyl)amino]sulfonyl}phenyl)-2-(2-phenylethyl)pentanoic acid

The compound was synthesized from methyl 5-[3-(tert-butoxycarbonyl(3,4-dimethoxyanilino)sulfonyl)phenyl]-2-(2-phenylethyl)pentanoate following the experimental procedure detailed in Method B. It was purified by flash chromatography on SiO₂ (10→60% EtOAc/hexanes) to yield a white solid (yield: 87%).

¹H NMR (CDCl₃, 250 MHz) δ ppm: 7.64 (bs, 1H), 7.57 (m, 1H), 7.42-7.31 (m, 4H), 7.29-7.17 (m, 3H), 7.10 (s, 1H), 6.77 (m, 2H), 6.61 (m, 1H), 3.86 (s, 3H), 3.82 (s, 3H), 2.69 (m, 4H), 2.50 (m, 1H), 2.06 (m, 1H), 1.90-1.49 (m, 5H).

EI MS: m/z=498 (M+1), 515 (M+18).

### Example 44

### 5-[3-(4-methylanilinosulfonyl)phenyl]-2-(3-phenylpropyl)pentanoic acid

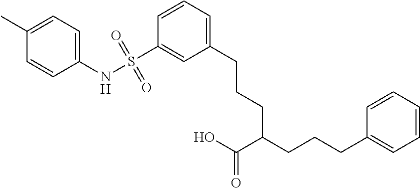

### a) Methyl 5-[3-(tert-butoxycarbonyl-4-methylanilinosulfonyl)phenyl]-2-(3-phenylpropyl)pent-4-ynoate

The compound was synthesized from methyl 5-phenylpentanoate and tert-butyl [3-(3-bromoprop-1-ynyl)phenyl]sulfonyl(4-methylphenyl)carbamate following the experimental procedure detailed in Method A. It was purified by flash chromatography on SiO₂ (10→15% EtOAc/hexanes) to furnish a yellow-coloured oil (yield: 72%).

¹H NMR (CDCl₃, 250 MHz) δ ppm: 8.09 (m, 1H), 7.97 (m, 1H), 7.67 (m, 1H), 7.55 (t, J=7.7 Hz, 1H), 7.38-7.21 (m, 9H), 3.81 (s, 3H), 2.74 (m, 5H), 2.47 (s, 3H), 1.79 (m, 4H), 1.44 (s, 9H).

### b) Methyl 5-[3-(tert-butoxycarbonyl-4-methylanilinosulfonyl)phenyl]-2-(3-phenylpropyl)pentanoate

The compound was synthesized from methyl 5-[3-(tert-butoxycarbonyl-4-methylanilinosulfonyl)phenyl]-2-(3-phenylpropyl)pent-4-ynoate following the experimental procedure detailed in Method E. The crude residue was submitted to next step without purification.

¹H NMR (CDCl₃, 250 MHz) δ ppm: 7.82 (m, 2H), 7.48 (m, 2H), 7.36-7.14 (m, 9H), 3.70 (s, 3H), 2.71 (m, 2H), 2.62 (m, 2H), 2.43 (s, 3H), 1.76-1.48 (m, 9H), 1.38 (s, 9H).

### c) 5-[3-(4-methylanilinosulfonyl)phenyl]-2-(3-phenylpropyl)pentanoic acid

The compound was prepared from methyl 5-[3-(tert-butoxycarbonyl-4-methylanilinosulfonyl)phenyl]-2-(3-phenylpropyl)pentanoate following the general procedure B. Flash chromatography purification on SiO₂ (20→50% EtOAc/hexanes), afforded the desired product as a yellow-coloured oil (yield: 73%).

¹H NMR (CDCl₃, 250 MHz) δ ppm: 7.66 (m, 2H), 7.41-7.17 (m, 8H), 7.09 (m, 4H), 2.70 (m, 4H), 2.48 (m, 1H), 2.34 (s, 3H), 1.92-1.36 (m, 8H).

EI MS: m/z=466 (M+1), 483 (M+18).

### Example 45

### 2-[2-(4-Ethylphenyl)ethyl]-5-phenylpentanoic acid

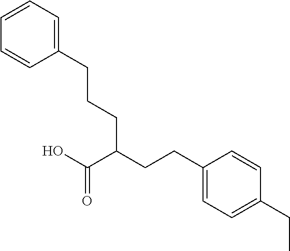

### a) Ethyl 4-(4-ethylphenyl)-4-hydroxybut-2-ynoate

Ethyl propiolate (470 mg, 4.85 mmol) was added to a −78° C. cooled solution of LiHMDS (5.2 mL, 1 M solution in THF, 5.22 mmol) in THF (15 mL). The reaction mixture was stirred at low temperature for 5 min, and 4-ethylbenzaldehyde (500 mg, 3.73 mmol) was added. It was allowed to reach r.t. and poured into H₂O (90 mL). It was taken up to pH=2 with HCl (10% aqueous solution), and the product was extracted with EtOAc (2×100 mL). The organic layer was dried over Na₂SO₄ (anhydrous), filtered and concentrated, to furnish a red oil, that was submitted to next step without purification.

### b) Ethyl 4-(4-ethylphenyl)butanoate

The compound was prepared from ethyl 4-(4-ethylphenyl)-4-hydroxybut-2-ynoate following the general procedure E. Flash chromatography purification on SiO₂ (0→10% EtOAc/hexanes) afforded ethyl 4-(4-ethylphenyl)butanoate as a colourless oil (yield: 75%).

¹H NMR (CDCl₃, 250 MHz) δ ppm: 7.12 (m, 4H), 4.12 (c, J=7.1 Hz, 2H), 2.60 (m, 4H), 2.34 (t, J=7.4 Hz, 2H), 1.96 (c, J=8.2 Hz, 2H), 1-32.1.20 (m, 6H).

### c) Ethyl 2-[2-(4-ethylphenyl)ethyl]-5-phenylpentanoate

The compound was prepared from ethyl 4-(4-ethylphenyl)butanoate following the general procedure A, by using PhCH₂CH₂CH₂I as alkylating reagent Flash chromatography purification on SiO₂ (5→10% EtOAc/hexanes) afforded ethyl 2-[2-(4-ethylphenyl)ethyl]-5-phenylpentanoate as a colourless oil (yield: 53%).

¹H NMR (CDCl₃, 250 MHz) δ ppm: 7.27 (m, 2H), 7.09 (m, 7H), 4.16 (c, J=7.1 Hz, 2H), 2.59 (m, 6H), 2.37 (m, 1H), 1.95 (m, 1H), 1.69-1.48 (m, 5H), 1.25 (m, 6H).

### d) 2-[2-(4-Ethylphenyl)ethyl]-5-phenylpentanoic acid

The compound was prepared from ethyl 2-[2-(4-ethylphenyl)ethyl]-5-phenylpentanoate following the general procedure B. Flash chromatography purification on SiO₂ (5→20% EtOAc/hexanes), afforded 2-[2-(4-ethylphenyl)ethyl]-5-phenylpentanoic acid as a colourless oil (yield: 25%).

¹H NMR (CDCl₃, 250 MHz) δ ppm: 7.47-7.34 (m, 9H), 2.88 (m, 7H), 2.22 (m, 1H), 2.07-1.73 (m, 5H), 1.47 (t, J=7.7 Hz, 3H).

EI MS: m/z=309 (M−1).

### Example 46

### 5-[4-(Anilinosulfonyl)phenyl]-2-[2-(4-ethylphenyl)ethyl]pentanoic acid

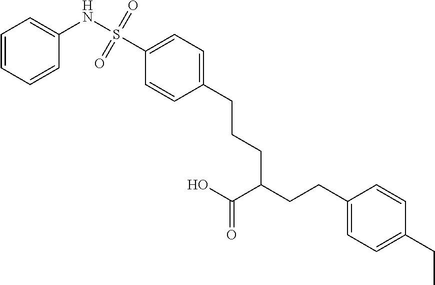

### a) Methyl 5-[4-(tert-butoxycarbonylanilinosulfonyl)phenyl]-2-[2-(4-ethylphenyl)ethyl]pent-4-ynoate

The compound was prepared from methyl 4-(4-ethylphenyl)butanoate following the general procedure A, by using tert-butyl[4-(3-bromoprop-1-ynyl)phenyl]sulfonyl(phenyl)carbamate as alkylating reagent. Flash chromatography purification on SiO₂ (5→15% EtOAc/hexanes) afforded the desired product as a yellow coloured oil (yield: 55%).

¹H NMR (CDCl₃, 250 MHz) δ ppm: 7.90 (d, J=7.4 Hz, 2H), 7.53 (d, J=7.4 Hz, 2H), 7.42 (m, 3H), 7.24 (m, 2H), 7.13 (m, 4H), 3.75 (s, 3H), 2.80-2.56 (m, 7H), 2.04 (m, 2H), 1.34 (s, 9H), 1.25 (c, J=7.1 Hz, 3H).

### b) Methyl 5-[4-(tert-butoxycarbonylanilinosulfonyl)phenyl]-2-[2-(4-ethylphenyl)ethyl]pentanoate

The compound was prepared from methyl 5-[4-(tert-butoxycarbonyl anilinosulfonyl)phenyl]-2-[2-(4-ethylphenyl)ethyl]pent-4-ynoate following the general procedure E. Flash chromatography purification on SiO₂ (20% EtOAc/hexanes) afforded the desired product as a colourless oil (yield: 88%).

¹H NMR (CDCl₃, 250 MHz) δ ppm: 7.88 (d, J=8.2 Hz, 2H), 7.42 (m, 3H), 7.34-7.22 (m, 4H), 7.09 (m, 4H), 3.68 (s, 3H), 2.73-2.35 (m, 7H), 1.94 (m, 1H), 1.81-1.48 (m, 5H), 1.33 (s, 9H), 1.21 (t, J=7.4 Hz).

### c) 5-[4-(Anilinosulfonyl)phenyl]-2-[2-(4-ethylphenyl)ethyl]pentanoic acid

The compound was prepared from methyl 5-[4-(tert-butoxycarbonylanilino sulfonyl)phenyl]-2-[2-(4-ethylphenyl)ethyl]pentanoate following the general procedure E. Flash chromatography purification on SiO₂ (15→70% EtOAc/hexanes) afforded 5-[4-(Anilinosulfonyl)phenyl]-2-[2-(4-ethylphenyl)ethyl]pentanoic acid as a colourless oil (yield: 62%).

¹H NMR (CDCl₃, 250 MHz) δ ppm: 7.62 (d, J=8.1 Hz, 2H), 7.36 (bs, 1H), 7.18-6.93 (m, 11H), 2.52 (m, 6H), 2.34 (m, 1H), 1.87 (m, 1H), 1.71-1.38 (m, 5H), 1.13 (t, J=7.6 Hz, 3H).

EI MS: m/z=466 (M+1), 483 (M+18).

### Example 47

### Methyl 5-[4-(anilinosulfonyl)phenyl]-2-[2-(4-ethylphenyl)ethyl]pentanoate

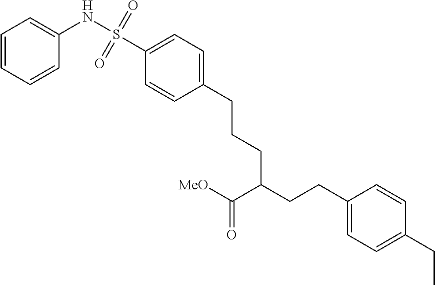

H₂SO₄ (360 mg, 0.373 mmol) was added to a solution of 5-[4-(anilinosulfonyl)phenyl]-2-[2-(4-ethylphenyl)ethyl]pentanoic acid (200 mg, 0.429 mmol) in MeOH (20 mL). The reaction mixture was allowed to react at r.t. for 10 h, poured into H₂O (150 mL) and extracted with CH₂Cl₂ (2×70 mL). The organic layer was dried over Na₂SO₄ (anhydrous), filtered and concentrated. The crude residue was flash chromatographed on SiO₂ (10→20% EtOAc/hexanes) to furnish 177 mg of methyl 5-[4-(anilinosulfonyl)phenyl]-2-[2-(4-ethylphenyl)ethyl]pentanoate (colourless oil, yield: 86%).

¹H NMR (CDCl₃, 250 MHz) δ ppm: 7.60 (d, J=8.5 Hz, 2H), 7.45 (bs, 1H), 7.13-6.88 (m, 11H), 3.55 (s, 3H), 2.44 (m, 6H), 2.28 (m, 1H), 1.81 (m, 1H), 1.65-1.29 (m, 5H), 1.10 (t, J=7.6 Hz, 3H).

EI MS: m/z=480 (M+1), 497 (M+18).

### Example 48

### 2-[2-(4-Ethylphenyl)ethyl]-6-phenylhexanoic acid

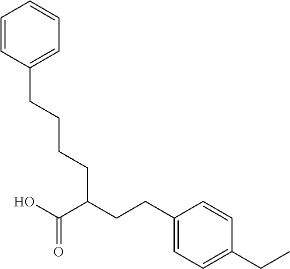

### a) Methyl 2-[2-(4-ethylphenyl)ethyl]-6-phenylhexanoate

The compound was synthesized from methyl 4-(4-ethylphenyl)butanoate and (4-iodobutyl)benzene following the experimental procedure detailed in Method A. It was purified by flash chromatography on SiO₂ (0→2% EtOAc/hexanes) to furnish a colourless oil (yield: 73%).

¹H NMR (CDCl₃, 250 MHz) δ ppm: 7.19 (m, 2H), 7.13-6.91 (m, 7H), 3.60 (s, 3H), 2.51 (m, 6H), 2.32 (m, 1H), 1.85 (m, 1H), 1.67-1.39 (m, 5H), 1.15 (m, 5H).

### b) 2-[2-(4-Ethylphenyl)ethyl]-6-phenylhexanoic acid

The compound was synthesized from methyl 2-[2-(4-ethylphenyl)ethyl]-6-phenylhexanoate following the experimental procedure detailed in Method B. The crude residue was purified by flash chromatography on SiO₂ (10→30% EtOAc/hexanes) to furnish a colourless oil (yield: 63%).

¹H NMR (CDCl₃, 250 MHz) δ ppm: 7.25 (m, 2H), 7.20-7.05 (m, 7H), 2.59 (m, 6H), 2.40 (m, 1H), 1.94 (m, 1H), 1.82-1.48 (m, 4H), 1.37 (m, 2H), 1.20 (m, 4H).

EI MS: m/z=342 (M+18).

### Example 49

### 5-(3-{[4-(Dimethylamino)benzoyl]amino}phenyl)-2-[2-(4-ethyl phenyl)ethyl]pentanoic acid

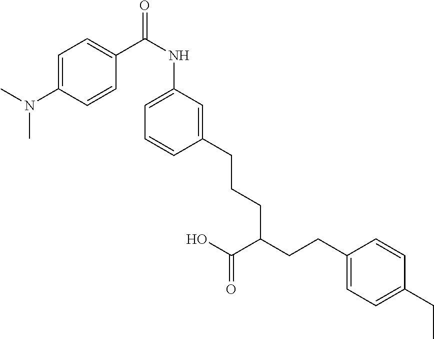

### a) Methyl 5-[3-N,N-dibenzylaminophenyl]-2-[2-(4-ethylphenyl)ethyl]pent-4-ynoate

The compound was synthesized from methyl 4-(4-ethylphenyl)butanoate and N,N-dibenzyl-N-[3-(3-bromoprop-1-ynyl)phenyl]amine following the experimental procedure detailed in Method A. It was purified by flash chromatography on SiO₂ (0→5% EtOAc/hexanes) to yield a yellow-coloured oil (yield: 67%).

¹H NMR (CDCl₃, 250 MHz) δ ppm: 7.42 (m, 11H), 7.22 (m, 4H), 6.91 (m, 3H), 4.79 (s, 4H), 3.81 (s, 3H), 2.79 (m, 8H), 2.15 (m, 1H), 1.40 (t, J=7.6 Hz, 3H).

### b) Methyl 5-(3-aminophenyl)-2-[2-(4-ethylphenyl)ethyl]pentanoate

The compound was synthesized from methyl 5-[3-N,N-dibenzylaminophenyl]-2-[2-(4-ethylphenyl)ethyl]pent-4-ynoate following the experimental procedure detailed in Method E. It was purified by flash chromatography on SiO₂ (10→30% EtOAc/hexanes) to yield a yellow-coloured oil (yield: 64%).

¹H NMR (CDCl₃, 250 MHz) δ ppm: 7.09 (m, 5H), 6.53 (m, 3H), 3.71 (s, 3H), 3.60 (bs, 2H), 2.51 (m, 6H), 1.94 (m, 1H), 1.78-1.43 (m, 6H), 1.20 (t, J=7.6 Hz, 3H).

### c) Methyl 5-(3-{[4-(dimethylamino)benzoyl]amino}phenyl)-2-[2-(4-ethylphenyl)ethyl]pentanoate

4-(Dimethylamino)benzoyl chloride (350 mg, 1.89 mmol) was added to a solution of methyl 5-(3-aminophenyl)-2-[2-(4-ethylphenyl)ethyl]pentanoate (630 mg, 1.58 mmol), DIPEA (0.87 mL, 5.06 mmol) and DMAP (30 mg, 0.246 mmol) in CH₂Cl₂ (40 mL). The reaction mixture was stirred at r.t. for 12 h, poured into H₂O (100 mL), and extracted with CH₂Cl₂ (100 mL). The organic layer was dried over Na₂SO₄ (anhydrous), filtered and concentrated. The crude residue was flash chromatographed on SiO₂ (10→30% EtOAc/hexanes) to furnish 615 mg of methyl 5-(3-{[4-(dimethylamino)benzoyl]amino}phenyl)-2-[2-(4-ethylphenyl)ethyl]pentanoate (yellow-coloured oil, yield: 80%).

¹H NMR (CDCl₃, 250 MHz) δ ppm: 7.79 (m, 2H), 7.70 (s, 1H), 7.46 (m, 2H), 7.23 (m, 1H), 7.09 (m, 4H), 6.87 (m, 1H), 6.70 (m, 2H), 3.68 (s, 3H), 3.06 (s, 6H), 2.70-2.35 (m, 7H), 1.91 (m, 1H), 1.80-1.49 (m, 5H), 1.22 (m, 3H).

### d) 5-(3-{[4-(Dimethylamino)benzoyl]amino}phenyl)-2-[2-(4-ethylphenyl)ethyl]pentanoic acid

The compound was synthesized from methyl 5-(3-{[4-(dimethylamino)benzoyl]amino}phenyl)-2-[2-(4-ethylphenyl)ethyl]pentanoate following the experimental procedure detailed in Method B. It was purified by flash chromatography on SiO₂ (10→40% EtOAc/hexanes) to yield a white solid (yield: 43%).

¹H NMR (CDCl₃, 250 MHz) δ ppm: 7.63 (m, 4H), 7.28 (m, 2H), 7.04 (m, 1H), 6.92 (m, 4H), 6.70 (m, 1H), 6.49 (d, J=8.8 Hz, 2H), 2.84 (s, 6H), 2.42 (m, 6H), 2.24 (m, 1H), 1.78 (m, 1H), 1.63-1.34 (m, 5H), 1.03 (t, J=7.4 Hz, 3H).

EI MS: m/z=473 (M+1).

### Example 50

### 5-(1-Naphthyl)-2-[2-(4-ethylphenyl)ethyl]-pentanoic acid

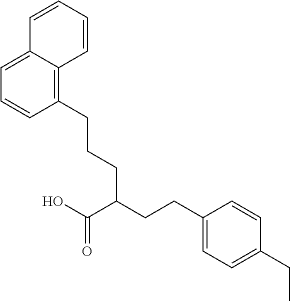

### a) Methyl 5-(1-naphthyl)-2-[2-(4-ethylphenyl)ethyl]-pent-4-ynoate

The compound was synthesized from methyl 4-(4-ethylphenyl)butanoate and 1-(3-bromoprop-1-ynyl)naphthalene following the experimental procedure detailed in Method A. It was purified by flash chromatography on SiO₂ (0→10% EtOAc/hexanes) to yield a yellow-coloured oil (yield: 42%).

EI MS: m/z=371 (M+1).

### b) Methyl 5-(1-naphthyl)-2-[2-(4-ethylphenyl)ethyl]-pentanoate

The compound was synthesized from methyl 5-(1-naphthyl)-2-[2-(4-ethylphenyl)ethyl]-pent-4-ynoate following the experimental procedure detailed in Method E. It was purified by flash chromatography on SiO₂ (2→4% EtOAc/hexanes) to yield a colourless oil (yield: 78%).

¹H NMR (CDCl₃, 250 MHz) δ ppm: 7.87 (m, 1H), 7.70 (m, 1H), 7.57 (m, 1H), 7.44-7.12 (m, 3H), 6.97 (m, 5H), 3.56 (s, 3H), 2.93 (m, 1H), 2.68-2.28 (m, 6H), 1.97-1.36 (m, 6H), 1.11 (t, J=7.6 Hz, 3H).

### c) 5-(1-Naphthyl)-2-[2-(4-ethylphenyl)ethyl]-pentanoic acid

The compound was synthesized from methyl 5-(1-naphthyl)-2-[2-(4-ethylphenyl)ethyl]-pentanoate following the experimental procedure detailed in Method B. It was purified by flash chromatography on SiO₂ (5→20% EtOAc/hexanes) to yield a colourless oil (yield: 14%).

¹H NMR (CDCl₃, 250 MHz) δ ppm: 7.90 (m, 1H), 7.75 (m, 1H), 7.61 (m, 1H), 7.43-7.15 (m, 4H), 6.99 (m, 4H), 2.96 (m, 2H), 2.59-2.28 (m, 5H), 1.90 (m, 1H), 1.81-1.45 (m, 5H), 1.14 (t, J=7.6 Hz, 3H).

EI MS: m/z=361 (M+1), 378 (M+18).

### Example 51

### 4-Phenyl-2-(2-phenylethyl)butanoic acid

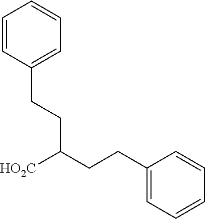

### a) Dimethyl bis(2-phenylethyl)malonate

NaH (760 mg, 60% mineral oil suspension, 19.0 mmol) was added to a solution of dimethyl malonate (1.0 g, 7.568 mmol) in DMF (40 mL). The mixture was stirred at r.t. for 15 min, and (2-bromoethyl)benzene (4.3 mL, 31.78 mmol) was added. The reaction mixture was warmed up to 50° C. and stirred for 4 h. It was allowed to reach r.t., diluted with NaCl (saturated aqueous solution, 200 mL) and extracted with Et₂O (200 mL). The organic layer was dried over Na₂SO₄ (anhydrous), filtered and concentrated. The crude residue was flash chromatographed on SiO₂ (2→10% EtOAc/hexanes) to furnish 1.023 g of dimethyl bis(2-phenylethyl)malonate (colourless oil, yield: 39%).

¹H NMR (CDCl₃, 250 MHz) δ ppm: 7.34 (m, 4H), 7.23 (m, 6H), 3.79 (s, 6H), 2.59 (m, 4H), 2.34 (m, 4H).

### b) bis(2-Phenylethyl)malonic acid

The compound was synthesized from dimethyl bis(2-phenylethyl)malonate following the experimental procedure detailed in Method B. It was purified by flash chromatography on SiO₂ (5 MeOH/CH₂Cl₂) to yield a white solid (yield: 25%). The compound was directly submitted to next step.

### c) 4-Phenyl-2-(2-phenylethyl)butanoic acid

H₂O (1 mL, 35.12 mmol) was added to a solution of bis(2-phenylethyl)malonic acid (300 mg, 0.878 mmol) in DMSO (25 mL), and the mixture was refluxed for 2 h. It was allowed to reach r.t., poured into H₂O (20 mL), taken up to pH=1 and extracted with EtOAc (20 mL). The organic layer was dried over Na₂SO₄ (anhydrous), filtered and concentrated. The crude residue was flash chromatographed on SiO₂ (10→20% EtOAc/hexanes) to furnish 70 mg of 4-phenyl-2-(2-phenylethyl)butanoic acid (yellow-coloured oil, yield: 30%).

¹H NMR (CDCl₃, 250 MHz) δ ppm: 7.35-7.08 (m, 10H), 2.67 (m, 5H), 2.46 (m, 1H), 2.01 (m, 2H), 1.80 (m, 2H).

EI MS: m/z=267 (M−1).

### Example 52

### 2-[2-(4-Bromophenyl)ethyl]-5-phenylpentanoic acid

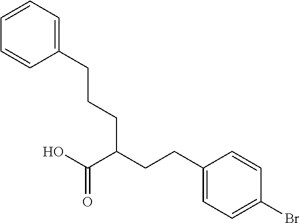

### a) Methyl 4-(4-bromophenyl)butanoate

H₂SO₄ (0.35 mL, 6.49 mmol) was added to a solution of 4-(4-bromophenyl)butanoic acid (5.26 g, 21.64 mmol) in MeOH (100 mL). The reaction mixture was stirred at r.t. for 16 h, and poured into H₂O (120 mL). It was extracted with CH₂Cl₂ (150 mL). The organic layer was dried over Na₂SO₄ (anhydrous), filtered and concentrated, to give 5.50 g of methyl 4-(4-bromophenyl)butanoate (colourless oil, yield: 98%). The compound was submitted to next step without further purification.

### b) Methyl 2-[2-(4-bromophenyl)ethyl]-5-phenylpentanoate

The compound was synthesized from methyl 4-(4-bromophenyl)butanoate and (3-iodopropyl)benzene following the experimental procedure detailed in Method A. It was purified by flash chromatography on SiO₂ (0→5% EtOAc/hexanes) to give a colourless oil (yield: 76%).

¹H NMR (CDCl₃, 250 MHz) δ ppm: 7.40 (m, 2H), 7.28 (m, 2H), 7.16 (m, 3H), 7.05 (m, 2H), 3.68 (s, 3H), 2.71-2.48 (m, 4H), 2.39 (m, 1H), 1.94 (m, 1H), 1.76-1.50 (m, 5H).

### c) 2-[2-(4-Bromophenyl)ethyl]-5-phenylpentanoic acid

The compound was synthesized from methyl 2-[2-(4-bromophenyl)ethyl]-5-phenylpentanoate following the experimental procedure detailed in Method B. It was purified by flash chromatography on SiO₂ (10→30% EtOAc/hexanes) to give a colourless oil (yield: 46%).

¹H NMR (CDCl₃, 250 MHz) δ ppm: 7.39 (m, 2H), 7.26 (m, 2H), 7.16 (m, 3H), 7.04 (m, 2H), 2.72-2.49 (m, 4H), 2.41 (m, 1H), 1.95 (m, 1H), 1.82-1.51 (m, 5H).

EI MS: m/z=359, 361 (M−1).

### Example 53

### 2-[2-(2,3-Dihydro-1H-indol-3-yl)ethyl]-5-phenylpentanoic acid

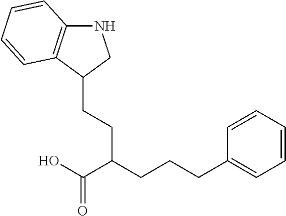

### a) Methyl 4-(1H-indol-3-yl)butanoate

H₂SO₄ (3.5 mL, 64.92 mmol) was added to a solution of indole-3-butyric acid (4.0 g, 19.68 mmol) in MeOH (100 mL). The reaction mixture was stirred at r.t. for 5 h, and poured into H₂O (150 mL). It was extracted with CH₂Cl₂ (2×100 mL). The organic layer was dried over Na₂SO₄ (anhydrous), filtered and concentrated, to give 4.22 g of methyl 4-(1H-indol-3-yl)butanoate (white solid, yield: 98%). The compound was submitted to next step without further purification.

¹H NMR (CDCl₃, 250 MHz) ppm: 7.97 (bs, 1H), 7.61 (d, J=7.7 Hz, 1H), 7.35 (d, J=8.0 Hz, 1H), 7.24-7.07 (m, 2H), 7.23 (bs, 1H), 3.66 (s, 3H), 2.81 (t, J=7.4 Hz, 2H), 2.40 (t, J=7.4 Hz, 2H), 2.06 (q, J=7.4 Hz, 2H).

### b) Methyl 4-(1-benzyl-1H-indol-3-yl)butanoate

Cs₂CO₃ (9.0 g, 27.62 mmol) was added to a solution of methyl 4-(1H-indol-3-yl)butanoate (4.0 g, 18.41 mmol) and BnBr (4.4 mL, 36.83 mmol) in CH₃CN (100 mL). The reaction mixture was refluxed for 14 h, allowed to reach r.t. and poured into H₂O (150 mL). It was extracted with EtOAc (180 mL). The organic layer was dried over Na₂SO₄ (anhydrous), filtered and concentrated. The crude residue was flash chromatographed on SiO₂ (20→25% EtOAc/hexanes) to furnish 2.94 g of methyl 4-(1-benzyl-1H-indol-3-yl)butanoate (colourless oil, yield: 53%).

¹H NMR (CDCl₃, 250 MHz) δ ppm: 7.56 (d, J=8.0 Hz, 1H), 7.31-6.98 (m, 8H), 6.85 (s, 1H), 5.18 (s, 2H), 3.60 (bs, 3H), 2.76 (t, J=7.4 Hz, 2H), 2.40 (t, J=7.4 Hz, 2H), 2.05-1.97 (m, 2H).

### c) (E)-Methyl 2-(2-(1-benzyl-1H-indol-3-yl)ethyl)-5-phenylpent-4-enoate

The compound was synthesized from methyl 4-(1-benzyl-1H-indol-3-yl)butanoate and [(1E)-3-bromoprop-1-enyl]benzene following the experimental procedure detailed in Method A. It was purified by flash chromatography on SiO₂ (0→5% EtOAc/hexanes) to give a colourless oil (yield: 46%).

### d) Methyl 2-(2-(indolin-3-yl)ethyl)-5-phenylpentanoate

The compound was synthesized from (E)-methyl 2-(2-(1-benzyl-1H-indol-3-yl)ethyl)-5-phenylpent-4-enoate following the experimental procedure detailed in Method E. It was purified by flash chromatography on SiO₂ (10→50% EtOAc/hexanes) to give a colourless oil (mixture of isomers, yield: 8%).

¹H NMR (CDCl₃, 250 MHz, signals of the major isomer) δ ppm: 7.41-7.11 (m, 8H), 6.76-6.59 (m, 1H), 3.67 (s, 3H), 3.29 (m, 1H), 3.27-3.11 (m, 2H), 2.65-2.53 (m, 2H), 2.46-2.26 (m, 3H), 1.79-1.36 (m, 7H).

### e) 2-[2-(2,3-Dihydro-1H-indol-3-yl)ethyl]-5-phenylpentanoic acid

The compound was synthesized from methyl 2-(2-(indolin-3-yl)ethyl)-5-phenylpentanoate following the experimental procedure detailed in Method B. It was purified by flash chromatography on SiO₂ (5% MeOH/CH₂Cl₂) to give a colourless oil (mixture of isomers, yield: 33%).

¹H NMR (CDCl₃, 250 MHz, signals of the major isomer) δ ppm: 7.26-7.03 (m, 5H), 7.03-6.89 (m, 2H), 6.72-6.52 (m, 3H), 3.73 (t, J=7.7 Hz, 1H), 3.38-3.17 (m, 2H), 2.69 (t, J=7.0 Hz, 2H), 2.47 (bs, 1H), 1.85-1.35 (m, 8H).

EI MS: m/z=324 (M+1).

### Example 54

### 2-[2-(4-hydroxyphenyl)ethyl]-5-phenylpentanoic acid

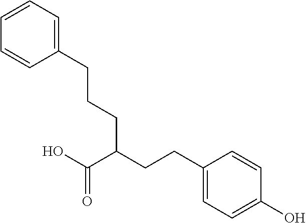

The compound was synthesized from methyl 2-[2-(4-hydroxyphenyl)ethyl]-5-phenylpentanoate following the experimental procedure detailed in Method B. It was purified by flash chromatography on SiO₂ (5→20% MeOH/CH₂Cl₂) to furnish, to afford a white solid (yield: 36%).

¹H NMR (CDCl₃, 250 MHz) δ ppm: 7.33 (m, 5H), 7.03 (d, J=8.2 Hz, 2H), 6.74 (d, J=8.2 Hz, 2H), 2.60 (m, 4H), 2.43 (m, 1H), 1.93 (m, 1H), 1.80-1.48 (m, 5H).

EI MS: m/z=297 (M−1), 316 (M+18).

### Example 55

### 2-[2-(4-Benzyloxyphenyl)ethyl]-5-phenylpentanoic acid

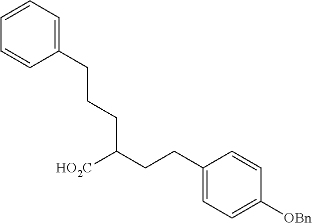

### a) Methyl 2-[2-(4-benzyloxyphenyl)ethyl]-5-phenylpentanoate

K₂CO₃ (1.40 mmol) was added to a solution of methyl 2-[2-(4-hydroxyphenyl)ethyl]-5-phenylpentanoate (1.60 g, 5.121 mmol) and BnBr (1 mL, 8.407 mmol) in CH₃CN (30 mL). The reaction mixture was stirred at r.t. for 6 h, poured into H₂O (120 mL) and extracted with EtOAc (150 mL). The organic layer was dried over Na₂SO₄ (anhydrous), filtered and concentrated. The crude residue was purified by flash chromatography on SiO₂ (10→20% EtOAc/hexanes), to give 1.73 g of methyl 2-[2-(4-benzyloxyphenyl)ethyl]-5-phenylpentanoate (colourless oil, yield: 84%).

¹H NMR (CDCl₃, 250 MHz) δ ppm: 7.44-7.19 (m, 7H), 7.18-7.08 (m, 3H), 7.03 (d, J=8.6 Hz, 2H), 6.86 (d, J=8.6 Hz, 2H), 5.01 (s, 2H), 3.64 (s, 3H), 2.60-2.32 (m, 5H), 1.90 (m, 1H), 1.76-1.44 (m, 5H).

### b) 2-[2-(4-Benzyloxyphenyl)ethyl]-5-phenylpentanoic acid

The compound was synthesized from methyl 2-[2-(4-benzyloxyphenyl)ethyl]-5-phenylpentanoate following the experimental procedure detailed in Method B. It was purified by flash chromatography on SiO₂ (15→30% EtOAc/hexanes) to furnish a white solid (yield: 58%).

¹H NMR (CDCl₃, 250 MHz) δ ppm: 7.32-7.04 (m, 7H), 7.03-6.88 (m, 5H), 6.72 (m, 2H), 4.86 (s, 2H), 2.42 (m, 4H), 2.24 (m, 1H), 1.78 (m, 1H), 1.50 (m, 5H).

EI MS: m/z=389 (M+1), 406 (M+18).

### Example 56

### (Acetyloxy)methyl 2-[2-(4-benzyloxyphenyl)ethyl]-5-phenyl pentanoate

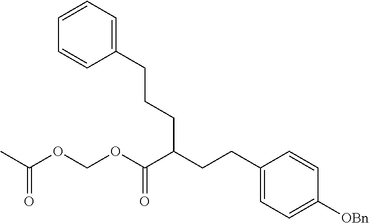

Bromomethyl acetate (0.055 mL, 0.564 mmol) was added to a solution of 2-[2-(4-benzyloxyphenyl)ethyl]-5-phenylpentanoic acid (Example 55) (160 mg, 0.412 mmol) and DIPEA (0.11 mL, 0.646 mmol) in CH₃CN (20 mL). The reaction mixture was stirred at r.t. for 3 h and solvent was concentrated off. The crude residue was flash chromatographed on SiO₂ (10→20% EtOAc/hexanes), to afford 134 mg of (acetyloxy)methyl 2-[2-(4-benzyloxyphenyl)ethyl]-5-phenylpentanoate (colourless oil, yield: 71%).

¹H NMR (CDCl₃, 250 MHz) δ ppm: 7.47-7.13 (m, 7H), 7.22-7.10 (m, 3H), 7.06 (d, J=8.8 Hz, 2H), 6.89 (d, J=8.8 Hz, 2H), 5.75 (s, 2H), 5.04 (s, 2H), 2.63-2.37 (m, 5H), 2.07 (s, 3H), 1.82-1.47 (m, 6H).

EI MS: m/z=478 (M+18).

### Example 57

### 2-[2-(4-Methoxyphenyl)ethyl]-5-phenylpentanoic acid

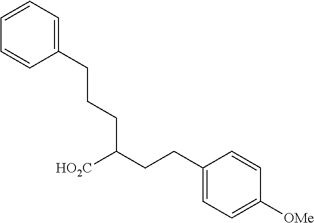

### a) Methyl 2-[2-(4-methoxyphenyl)ethyl]-5-phenylpentanoate

K₂CO₃ (1.40 mmol) was added to a solution of methyl 2-[2-(4-hydroxyphenyl)ethyl]-5-phenylpentanoate (1.60 g, 5.121 mmol) and MeI (1.20 g, 8.407 mmol) in CH₃CN (30 mL). The reaction mixture was stirred at r.t. for 6 h, poured into H₂O (120 mL) and extracted with EtOAc (150 mL). The organic layer was dried over Na₂SO₄ (anhydrous), filtered and concentrated. The crude residue was purified by flash chromatography on SiO₂ (10% EtOAc/hexanes), to give 1.55 g of methyl 2-[2-(4-methoxyphenyl)ethyl]-5-phenylpentanoate (white solid, yield: 93%).

¹H NMR (CDCl₃, 250 MHz) δ ppm: 7.32-7.11 (m, 5H), 7.07 (d, J=8.6 Hz, 2H), 6.82 (d, J=8.6 Hz, 2H), 3.79 (s, 3H), 3.69 (s, 3H), 2.65-2.34 (m, 5H), 1.91 (m, 1H), 1.78-1.40 (m, 5H).

### b) 2-[2-(4-Methoxyphenyl)ethyl]-5-phenylpentanoic acid

The compound was synthesized from methyl 2-[2-(4-methoxyphenyl)ethyl]-5-phenylpentanoate following the experimental procedure detailed in Method B. It was purified by flash chromatography on SiO₂ (0→5% EtOAc/hexanes) to furnish a yellow-coloured oil (yield: 58%).

¹H NMR (CDCl₃, 250 MHz) δ ppm: 7.31-7.12 (m, 5H), 7.09 (d, J=8.6 Hz, 2H), 6.81 (d, J=8.6 Hz, 2H), 3.78 (s, 3H), 2.62 (m, 4H), 2.42 (m, 1H), 1.96 (m, 1H), 1.83-1.50 (m, 5H).

EI MS: m/z=313 (M+1), 330 (M+18).

### Example 58

### 2-{2-[4-(Pyridin-2-ylmethoxy)phenyl]ethyl}-5-phenylpentanoic acid

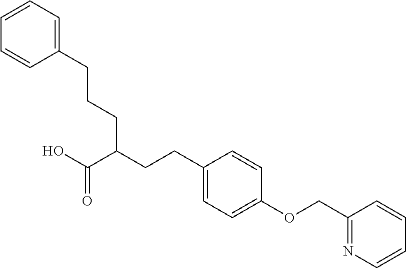

### a) Methyl 2-{2-[4-(pyridin-2-ylmethoxy)phenyl]ethyl}-5-phenylpentanoate

2-(Bromomethyl)pyridine hydrobromide (425 mg, 1.68 mmol) was added to a suspension of methyl 2-[2-(4-hydroxyphenyl)ethyl]-5-phenylpentanoate (400 mg, 1.28 mmol) and Cs₂CO₃ (1.30 g, 3.99 mmol) in DMF (25 mL). The reaction mixture was warmed up to 60° C. and stirred for 3 h. It was allowed to reach r.t. and poured into H₂O (150 mL), taken up to pH=3 with HCl and extracted with Et₂O (100 mL). The organic layer was dried over Na₂SO₄ (anhydrous), filtered and concentrated. The crude residue was purified by flash chromatography on SiO₂ (10→30% EtOAc/hexanes), to give 452 mg of methyl 2-{2-[4-(pyridin-2-ylmethoxy)phenyl]ethyl}-5-phenylpentanoate (colourless oil, yield: 88%).

¹H NMR (CDCl₃, 250 MHz) δ ppm: 8.69 (d, J=4.9 Hz, 1H), 7.80 (m, 1H), 7.62 (m, 1H), 7.42-7.21 (m, 6H), 7.17 (m, 2H), 7.00 (m, 2H), 5.29 (s, 2H), 3.77 (s, 3H), 2.72-2.42 (m, 5H), 2.00 (m, 1H), 1.88-1.53 (m, 5H).

### b) 2-{2-[4-(Pyridin-2-ylmethoxy)phenyl]ethyl}-5-phenylpentanoic acid

The compound was synthesized from methyl 2-{2-[4-(pyridin-2-ylmethoxy)phenyl]ethyl}-5-phenylpentanoate following the experimental procedure detailed in Method B. The crude residue was slurred with Et₂O, to afford a white solid (yield: 76%).

¹H NMR (CDCl₃, 250 MHz) δ ppm: 8.62 (d, J=6.0 Hz, 1H), 7.75 (m, 1H), 7.57 (m, 1H), 7.28-7.00 (m, 8H), 6.89 (d, J=8.5 Hz, 2H), 5.21 (s, 2H), 2.57 (m, 4H), 2.41 (m, 1H), 1.91 (m, 1H), 1.80-1.44 (m, 5H).

EI MS: m/z=390 (M+1).

### Example 59

### 2-{2-[4-(Pyridin-3-ylmethoxy)phenyl]ethyl}-5-phenyl pentanoic acid

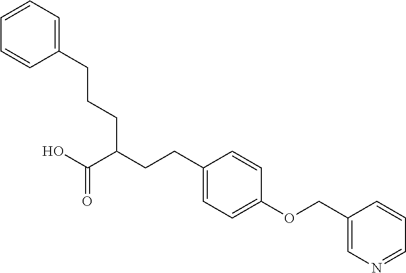

### a) Methyl 2-{2-[4-(pyridin-3-ylmethoxy)phenyl]ethyl}-5-phenylpentanoate

3-(Bromomethyl)pyridine hydrobromide (260 mg, 1.027 mmol) was added to a suspension of methyl 2-[2-(4-hydroxyphenyl)ethyl]-5-phenylpentanoate (250 mg, 0.800 mmol) and Cs₂CO₃ (820 mg, 2.516 mmol) in DMF (20 mL). The reaction mixture was warmed up to 60° C. and stirred for 6 h. It was allowed to reach r.t. and poured into H₂O (150 mL), taken up to pH=3 with HCl and extracted with Et₂O (120 mL). The organic layer was dried over Na₂SO₄ (anhydrous), filtered and concentrated. The crude residue was purified by flash chromatography on SiO₂ (10→80% EtOAc/hexanes), to give 85 mg of methyl 2-{2-[4-(pyridin-3-ylmethoxy)phenyl]ethyl}-5-phenylpentanoate (colourless oil, yield: 26%).

¹H NMR (CDCl₃, 250 MHz) δ ppm: 8.66 (bs, 1H), 8.57 (bs, 1H), 7.75 (d, J=7.9 Hz, 1H), 7.25 (m, 3H), 7.20-7.03 (m, 5H), 6.87 (m, 2H), 5.03 (s, 2H), 3.66 (s, 3H), 2.55 (m, 4H), 2.38 (m, 1H), 1.90 (m, 1H), 1.78-1.45 (m, 5H).

### b) 2-{2-[4-(Pyridin-3-ylmethoxy)phenyl]ethyl}-5-phenylpentanoic acid

The compound was synthesized from methyl 2-{2-[4-(pyridin-3-ylmethoxy)phenyl]ethyl}-5-phenylpentanoate following the experimental procedure detailed in Method B. It was purified by flash chromatography on SiO₂ (30→50% MeOH/CH₂Cl₂) to afford a white solid (yield: 22%).

¹H NMR (MeOD, 250 MHz) δ ppm: 8.63 (bs, 1H), 8.51 (bs, 1H), 7.94 (d, J=7.7 Hz, 1H), 7.45 (m, 1H), 7.28-7.02 (m, 7H), 6.95 (m, 2H), 5.12 (s, 2H), 2.56 (m, 4H), 2.31 (m, 1H), 1.87 (m, 1H), 1.72-1.42 (m, 5H).

EI MS: m/z=390 (M+1).

### Example 60

### 2-{2-[4-(Pyridin-4-ylmethoxy)phenyl]ethyl}-5-phenylpentanoic acid

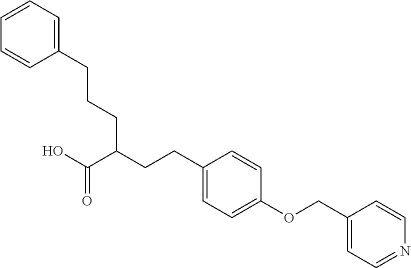

### a) Methyl 2-{2-[4-(pyridin-4-ylmethoxy)phenyl]ethyl}-5-phenylpentanoate

4-(Bromomethyl)pyridine hydrobromide (500 mg, 1.976 mmol) was added to a suspension of methyl 2-[2-(4-hydroxyphenyl)ethyl]-5-phenylpentanoate (400 mg, 1.28 mmol) and Cs₂CO₃ (1.30 g, 3.98 mmol) in DMF (20 mL). The reaction mixture was warmed up to 60° C. and stirred for 3 h. It was allowed to reach r.t. and poured into H₂O (150 mL), taken up to pH=3 with HCl and extracted with Et₂O (100 mL). The organic layer was dried over Na₂SO₄ (anhydrous), filtered and concentrated. The crude residue was purified by flash chromatography on SiO₂ (10→80% EtOAc/hexanes), to give 245 mg of methyl 2-{2-[4-(pyridin-4-ylmethoxy)phenyl]ethyl}-5-phenylpentanoate (colourless oil, yield: 49%).

¹H NMR (CDCl₃, 250 MHz) δ ppm: 8.60 (m, 2H), 7.34 (m, 2H), 7.26 (m, 2H), 7.21-7.03 (m, 5H), 6.85 (m, 2H), 5.07 (s, 2H), 3.67 (s, 3H), 2.61-2.33 (m, 5H), 1.90 (m, 1H), 1.75-1.46 (m, 5H).

### b) 2-{2-[4-(Pyridin-4-ylmethoxy)phenyl]ethyl}-5-phenylpentanoic acid

The compound was synthesized from methyl 2-{2-[4-(pyridin-4-ylmethoxy)phenyl]ethyl}-5-phenylpentanoate following the experimental procedure detailed in Method B. It was purified by flash chromatography on SiO₂ (5→20% MeOH/CH₂Cl₂) to afford a white solid (yield: 36%).

¹H NMR (CDCl₃, 250 MHz) δ ppm: 11.23 (m, 2H), 8.85 (m, 2H), 7.67 (m, 2H), 7.57-7.15 (m, 7H), 7.05 (d, J=8.2 Hz, 2H), 5.27 (s, 2H), 2.81 (m, 4H), 2.63 (m, 1H), 2.15 (m, 1H), 2.07-1.63 (m, 5H).

EI MS: m/z=390 (M+1).

### Example 61

### 2-{2-[4-(2-Cyanophenoxy)phenyl]ethyl}-5-phenylpentanoic acid

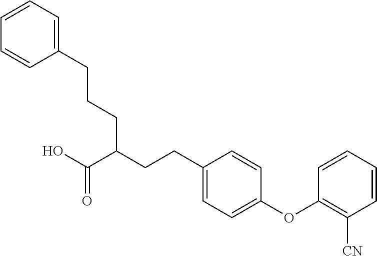

### a) Methyl 2-{2-[4-(2-cyanophenoxy)phenyl]ethyl}-5-phenylpentanoate

K₂CO₃ (200 mg, 1.447 mmol) was added to a solution of methyl 2-[2-(4-hydroxyphenyl)ethyl]-5-phenylpentanoate (200 mg, 0.640 mmol) and 2-fluorobenzonitrile (200 mg, 1.651 mmol) in DMF (20 mL). The reaction mixture was warmed up to 130° C., and allowed to react for 30 min. It was poured into H₂O (120 mL) and extracted with Et₂O (100 mL). The organic layer was dried over Na₂SO₄ (anhydrous), filtered and concentrated. The crude residue was purified by flash chromatography on SiO₂ (4→16% EtOAc/hexanes), to give 232 mg of methyl 2-{2-[4-(2-cyanophenoxy)phenyl]ethyl}-5-phenylpentanoate (colourless oil, yield: 88%).

¹H NMR (CDCl₃, 250 MHz) δ ppm: 7.69 (dd, J=7.9 Hz, 1.9 Hz, 1H), 7.50 (m, 1H), 7.36-7.11 (m, 8H), 7.04 (m, 2H), 6.87 (d, J=7.9 Hz, 1H), 3.74 (s, 3H), 2.64 (m, 4H), 2.45 (m, 1H), 2.00 (m, 1H), 1.85-1.54 (m, 5H).

### b) 2-{2-[4-(2-Cyanophenoxy)phenyl]ethyl}-5-phenylpentanoic acid

The compound was synthesized from methyl 2-{2-[4-(2-cyanophenoxy)phenyl]ethyl}-5-phenylpentanoate following the experimental procedure detailed in Method B. It was purified by flash chromatography on SiO₂ (10→40% EtOAc/hexanes) to furnish a colourless oil (yield: 72%).

¹H NMR (CDCl₃, 250 MHz) δ ppm: 7.72 (m, 1H), 7.51 (m, 1H), 7.38-7.12 (m, 8H), 7.07 (d, J=8.5 Hz, 2H), 6.90 (d, J=8.5 Hz, 1H), 2.71 (m, 4H), 2.51 (m, 1H), 2.08 (m, 1H), 1.95-1.58 (m, 5H).

EI MS: m/z=398 (M−1), 400 (M+1).

### Example 62

### 2-{2-[4-(3-cyanophenoxy)phenyl]ethyl}-5-phenylpentanoic acid

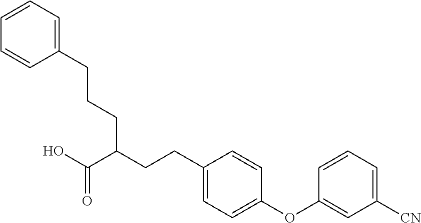

### a) Methyl 2-{2-[4-(3-cyanophenoxy)phenyl]ethyl}-5-phenylpentanoate

K₂CO₃ (250 mg, 1.808 mmol) was added to a solution of methyl 2-[2-(4-hydroxyphenyl)ethyl]-5-phenylpentanoate (250 mg, 0.800 mmol) and 3-fluorobenzonitrile (250 mg, 2.064 mmol) in DMF (25 mL). The reaction mixture was warmed up to 130° C., and allowed to react for 6 h. It was poured into H₂O (120 mL) and extracted with Et₂O (100 mL). The organic layer was dried over Na₂SO₄ (anhydrous), filtered and concentrated. The crude residue was purified by flash chromatography on SiO₂ (5→15% EtOAc/hexanes), to give 142 mg of methyl 2-{2-[4-(3-cyanophenoxy)phenyl]ethyl}-5-phenylpentanoate (colourless oil, yield: 43%).

¹H NMR (CDCl₃, 250 MHz) δ ppm: 7.28-6.91 (m, 11H), 6.78 (dd, J=8.2 Hz, 1.3 Hz, 2H), 3.54 (s, 3H), 2.40 (m, 4H), 2.28 (m, 1H), 1.78 (m, 1H), 1.69-1.28 (m, 5H).

### b) 2-{2-[4-(3-cyanophenoxy)phenyl]ethyl}-5-phenylpentanoic acid

The compound was synthesized from methyl 2-{2-[4-(3-cyanophenoxy)phenyl]ethyl}-5-phenylpentanoate following the experimental procedure detailed in Method B. It was purified by flash chromatography on SiO₂ (30→40% EtOAc/hexanes) to furnish a colourless oil (yield: 50%).

¹H NMR (CDCl₃, 250 MHz) δ ppm: 7.59 (m, 1H), 7.48-6.97 (m, 10H), 6.86 (d, J=8.5 Hz, 2H), 2.53 (m, 4H), 2.37 (m, 1H), 1.91 (m, 1H), 1.78-1.41 (m, 5H).

EI MS: m/z=398 (M−1), 400 (M+1).

### Example 63

### 2-{2-[4-(4-cyanophenoxy)phenyl]ethyl}-5-phenylpentanoic acid

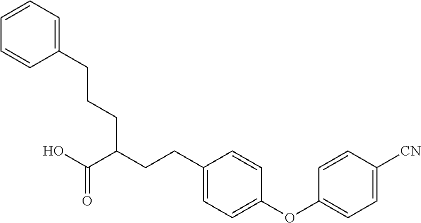

### a) Methyl 2-{2-[4-(4-cyanophenoxy)phenyl]ethyl}-5-phenylpentanoate

K₂CO₃ (100 mg, 0.723 mmol) was added to a solution of methyl 2-[2-(4-hydroxyphenyl)ethyl]-5-phenylpentanoate (100 mg, 0.320 mmol) and 4-fluorobenzonitrile (100 mg, 0.825 mmol) in DMF (15 mL). The reaction mixture was warmed up to 130° C., and allowed to react for 2 h. It was poured into H₂O (120 mL) and extracted with Et₂O (100 mL). The organic layer was dried over Na₂SO₄ (anhydrous), filtered and concentrated. The crude residue was purified by flash chromatography on SiO₂ (5→15% EtOAc/hexanes), to give 133 mg of methyl 2-{2-[4-(4-cyanophenoxy)phenyl]ethyl}-5-phenylpentanoate (colourless oil, yield: 99%).

¹H NMR (CDCl₃, 250 MHz) δ ppm: 7.66 (m, 2H), 7.41-7.20 (m, 7H), 7.06 (m, 4H), 3.79 (s, 3H), 2.67 (m, 4H), 2.50 (m, 1H), 2.05 (m, 1H), 1.97-1.60 (m, 5H).

### b) 2-{2-[4-(4-cyanophenoxy)phenyl]ethyl}-5-phenylpentanoic acid

The compound was synthesized from methyl 2-{2-[4-(4-cyanophenoxy)phenyl]ethyl}-5-phenylpentanoate following the experimental procedure detailed in Method B. It was purified by flash chromatography on SiO₂ (5→40% EtOAc/hexanes) to furnish a colourless oil (yield: 54%).

¹H NMR (CDCl₃, 250 MHz) δ ppm: 7.48 (m, 2H), 7.23-6.99 (m, 7H), 6.88 (m, 4H), 2.55 (m, 4H), 2.36 (m, 1H), 1.91 (m, 1H), 1.77-1.43 (m, 5H).

EI MS: m/z=398 (M−1), 400 (M+1).

### Example 64

### 2-{2-[4-(4-Fluorophenoxy)phenyl]ethyl}-5-phenylpentanoic acid

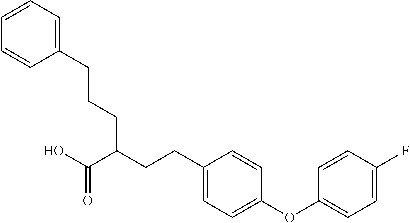

Cu(OAc)₂ (150 mg, 0.825 mmol) was added to a solution of methyl 2-[2-(4-hydroxyphenyl)ethyl]-5-phenylpentanoate (100 mg, 0.320 mmol), pyridine (0.15 mL, 1.854 mmol) and 4-fluorophenylboronic acid (90 mg, 0.643 mmol) in CH₂Cl₂ (25 mL). The reaction mixture was stirred at r.t. for 20 h. It was poured into H₂O (150 mL), taken up to pH=2 and extracted with CH₂Cl₂ (100 mL). The organic layer was dried over Na₂SO₄ (anhydrous), filtered and concentrated. The crude residue was purified by flash chromatography on SiO₂ (2→8% EtOAc/hexanes), to give 122 mg of methyl 2-{2-[4-(4-fluoro phenoxy)phenyl]ethyl}-5-phenylpentanoate (colourless oil, yield: 94%).

¹H NMR (CDCl₃, 250 MHz) δ ppm: 7.39 (m, 2H), 7.34-7.20 (m, 5H), 7.18-6.95 (m, 6H), 3.82 (s, 3H), 2.69 (m, 4H), 2.55 (m, 1H), 2.08 (m, 1H), 1.94-1.58 (m, 5H).

### b) 2-{2-[4-(4-Fluorophenoxy)phenyl]ethyl}-5-phenylpentanoic acid

The compound was synthesized from methyl 2-{2-[4-(4-fluorophenoxy)phenyl]ethyl}-5-phenylpentanoate following the experimental procedure detailed in Method B. It was purified by flash chromatography on SiO₂ (10→80% EtOAc/hexanes) to furnish a colourless oil (yield: 62%).

¹H NMR (CDCl₃, 250 MHz) δ ppm: 7.25 (m, 2H), 7.21-7.07 (m, 5H), 7.01-6.83 (m, 6H), 2.60 (m, 4H), 2.40 (m, 1H), 1.96 (m, 1H), 1.84-1.47 (m, 5H).

EI MS: m/z=391 (M−1).

### Example 65

### 2-{2-[4-Phenoxyphenyl]ethyl}-5-phenylpentanoic acid

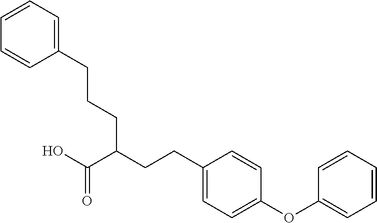

### a) Methyl 2-{2-[4-phenoxyphenyl]ethyl}-5-phenylpentanoate

Cu(OAc)₂ (232 mg, 1.28 mmol) was added to a solution of methyl 2-[2-(4-hydroxyphenyl)ethyl]-5-phenylpentanoate (160 mg, 0.512 mmol), pyridine (0.21 mL, 2.56 mmol) and phenylboronic acid (123 mg, 1.02 mmol) in CH₂Cl₂ (25 mL). The reaction mixture was stirred at r.t. for 20 h. It was poured into H₂O (150 mL), taken up to pH=2 and extracted with CH₂Cl₂ (2×20 mL). The organic layer was dried over Na₂SO₄ (anhydrous), filtered and concentrated. The crude residue was purified by flash chromatography on SiO₂ (0→8% EtOAc/hexanes), to give 119 mg of methyl 2-{2-[4-phenoxyphenyl]ethyl}-5-phenylpentanoate (colourless oil, yield: 60%).

¹H NMR (CDCl₃, 250 MHz) δ ppm: 7.30 (m, 5H), 7.15 (m, 5H), 6.98 (m, 4H), 3.70 (s, 3H), 2.57 (m, 4H), 2.46 (m, 1H), 1.96 (m, 1H), 1.83-1.48 (m, 5H).

### b) 2-{2-[4-Phenoxyphenyl]ethyl}-5-phenylpentanoic acid

The compound was synthesized from methyl 2-{2-[4-phenoxyphenyl]ethyl}-5-phenylpentanoate following the experimental procedure detailed in Method B. It was purified by flash chromatography on SiO₂ (10→80% EtOAc/hexanes) to furnish a colourless oil (yield: 12%).

¹H NMR (CDCl₃, 250 MHz) δ ppm: 7.40-6.90 (m, 14H), 2.63 (m, 4H), 2.48 (m, 1H), 2.01 (m, 1H), 1.87-1.50 (m, 5H).

EI MS: m/z=373 (M−1).

### Example 66

### 2-{2-[4-(4-Trifluoromethylphenoxy)phenyl]ethyl}-5-phenyl pentanoic acid

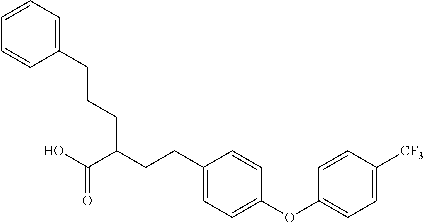

### a) Methyl 2-{2-[4-(4-trifluoromethylphenoxy)phenyl]ethyl}-5-phenylpentanoate

Cu(OAc)₂ (232 mg, 1.28 mmol) was added to a solution of methyl 2-[2-(4-hydroxyphenyl)ethyl]-5-phenylpentanoate (160 mg, 0.512 mmol), pyridine (0.21 mL, 2.56 mmol) and 4-(trifluoromethyl)phenylboronic acid (193 mg, 1.02 mmol) in CH₂Cl₂ (25 mL). The reaction mixture was stirred at r.t. for 20 h. It was poured into H₂O (150 mL), taken up to pH=2 and extracted with CH₂Cl₂ (2×20 mL). The organic layer was dried over Na₂SO₄ (anhydrous), filtered and concentrated. The crude residue was purified by flash chromatography on SiO₂ (0→8% EtOAc/hexanes), to give 200 mg of methyl 2-{2-[4-(4-trifluoromethylphenoxy)phenyl]ethyl}-5-phenylpentanoate (colourless oil, yield: 60%).

¹H NMR (CDCl₃, 250 MHz) δ ppm: 7.56 (m, 2H), 7.37-6.84 (m, 11H), 3.71 (s, 3H), 2.66-2.35 (m, 5H), 1.95 (m, 1H), 1.83-1.42 (m, 5H).

### b) 2-{2-[4-(4-Trifluoromethylphenoxy)phenyl]ethyl}-5-phenylpentanoic acid

The compound was synthesized from methyl 2-{2-[4-(4-trifluoromethyl phenoxy)phenyl]ethyl}-5-phenylpentanoate following the experimental procedure detailed in Method B. It was purified by flash chromatography on SiO₂ (10→80% EtOAc/hexanes) to furnish a colourless oil (yield: 36%).

¹H NMR (CDCl₃, 250 MHz) δ ppm: 7.56 (d, J=9.0 Hz, 2H), 7.37-7.13 (m, 8H), 7.00 (m, 3H), 2.67 (m, 4H), 2.47 (m, 1H), 2.00 (m, 1H), 1.86-1.53 (m, 5H).

EI MS: m/z=441 (M−1).

### Example 67

### 2-{2-[4-(4-Methoxyphenoxy)phenyl]ethyl}-5-phenylpentanoic acid

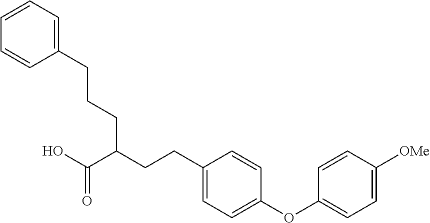

### a) Methyl 2-{2-[4-(4-methoxyphenoxy)phenyl]ethyl}-5-phenylpentanoate

Cu(OAc)₂ (232 mg, 1.28 mmol) was added to a solution of methyl 2-[2-(4-hydroxyphenyl)ethyl]-5-phenylpentanoate (160 mg, 0.512 mmol), pyridine (0.21 mL, 2.56 mmol) and 4-(methoxy)phenylboronic acid (155 mg, 1.02 mmol) in CH₂Cl₂ (25 mL). The reaction mixture was stirred at r.t. for 20 h. It was poured into H₂O (150 mL), taken up to pH=2 and extracted with CH₂Cl₂ (2×20 mL). The organic layer was dried over Na₂SO₄ (anhydrous), filtered and concentrated. The crude residue was purified by flash chromatography on SiO₂ (0→8% EtOAc/hexanes), to give 110 mg of methyl 2-{2-[4-(4-methoxyphenoxy)phenyl]ethyl}-5-phenylpentanoate (colourless oil, yield: 52%).

¹H NMR (CDCl₃, 250 MHz) δ ppm: 7.30-6.99 (m, 9H), 6.98-6.75 (m, 4H), 3.77 (s, 3H), 3.66 (s, 3H), 2.57 (m, 4H), 2.38 (m, 1H), 1.76-1.47 (m, 5H).

### b) 2-{2-[4-(4-Methoxyphenoxy)phenyl]ethyl}-5-phenylpentanoic acid

The compound was synthesized from methyl 2-{2-[4-(4-methoxyphenoxy)phenyl]ethyl}-5-phenylpentanoate following the experimental procedure detailed in Method B. It was purified by flash chromatography on SiO₂ (0→5% EtOAc/hexanes) to furnish a colourless oil (yield: 69%).

¹H NMR (CDCl₃, 250 MHz) δ ppm: 7.40.7.14 (m, 8H), 7.09-6.90 (m, 5H), 3.88 (s, 3H), 2.70 (m, 4H), 2.52 (m, 1H), 2.03 (m, 1H), 1.91-1.56 (m, 5H).

EI MS: m/z=403 (M−1).

### Example 68

### 4-Phenyl-2-[(2-phenylethyl)thio]butanoic acid

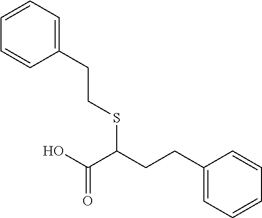

### a) Ethyl 2-hydroxy-4-phenylbutanoate

NaBH₄ (625 mg, 16.521 mmol) was added to a −18° C. cooled solution of ethyl 2-oxo-4-phenylbutyrate in EtOH (70 mL). The reaction mixture was stirred at low temperature for 5 min, poured into H₂O (120 mL), taken up to pH=2 with HCl and extracted with CH₂Cl₂ (200 mL). The organic layer was dried over Na₂SO₄ (anhydrous), filtered and concentrated. The crude residue was purified by flash chromatography on SiO₂ (5→15% EtOAc/hexanes), to give 2.85 g of ethyl 2-hydroxy-4-phenylbutanoate (colourless oil, yield: 83%).

### b) Ethyl 4-phenyl-2-{[(trifluoromethyl)sulfonyl]oxy}butanoate

Trifluoromethanesulfonic anhydride (1.70 g, 6.025 mmol) was added to a −78° C. cooled solution of ethyl 2-hydroxy-4-phenylbutanoate (1.0 g, 4.801 mmol) and 2,6-luitidine (1.7 mL, 14.642 mmol) in CH₂Cl₂ (40 mL). The reaction mixture was allowed to react at low temperature for 5 min, poured into H₂O (150 mL), taken up to pH=3 with HCl and extracted with CH₂Cl₂ (150 mL). The organic layer was dried over Na₂SO₄ (anhydrous), filtered and concentrated. The crude residue was purified by flash chromatography on SiO₂ (4→10% EtOAc/hexanes), to give 1.41 g of ethyl 4-phenyl-2-{[(trifluoromethyl)sulfonyl]oxy}butanoate (colourless oil, yield: 86%).

EI MS: m/z=341 (M+1).

### c) Ethyl 4-phenyl-2-[(2-phenylethyl)thio]butanoate

Benzeneethanethiol (0.45 mL, 3.33 mmol) was added to a solution of ethyl 4-phenyl-2-{[(trifluoromethyl)sulfonyl]oxy}butanoate (750 mg, 2.20 mmol) and DIPEA (1.5 mL, 8.76 mmol) in CH₂Cl₂ (50 mL). The reaction mixture stirred at r.t. for 2 h, poured into H₂O (200 mL) and extracted with Et₂O (100 mL). The organic layer was dried over Na₂SO₄ (anhydrous), filtered and concentrated. The crude residue was purified by flash chromatography on SiO₂ (0 4% EtOAc/hexanes), to give 635 mg of ethyl 4-phenyl-2-[(2-phenylethyl)thio]butanoate (colourless oil, yield: 88%).

¹H NMR (CDCl₃, 250 MHz) δ ppm: 7.62-7.34 (m, 10H), 4.46 (c, J=7.1 Hz, 2H), 3.50 (t, J=7.1 Hz, 1H), 3.27-2.93 (m, 6H), 2.48 (m, 1H), 2.24 (m, 1H), 1.55 (t, J=7.1 Hz, 3H).

### d) 4-Phenyl-2-[(2-phenylethyl)thio]butanoic acid

The compound was synthesized from ethyl 4-phenyl-2-[(2-phenylethyl)thio]butanoate following the experimental procedure detailed in Method B. It was purified by flash chromatography on SiO₂ (5→40% EtOAc/hexanes) to afford a colourless oil (yield: 56%).

¹H NMR (CDCl₃, 250 MHz) δ ppm: 7.36-7.09 (m, 10H), 3.22 (t, J=7.4 Hz, 1H), 2.90 (m, 4H), 3.22 (t, J=7.6 Hz, 2H), 2.21 (m, 1H), 1.99 (m, 1H).

EI MS: m/z=299 (M−1).

### Example 69

### 2-(Benzyloxy)-5-phenylpentanoic acid

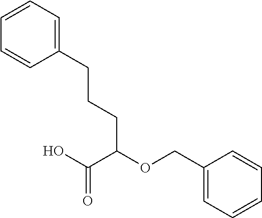

### a) Ethyl 2-oxo-5-phenylpentanoate

A solution of (3-bromopropyl)benzene (2.0 g, 10.045 mmol) in Et₂O (10 mL) was dropwise was added to a refluxing suspension of Mg (powder, 240 mg, 9.872 mmol) and 1,2-dibromoethane (drops) in Et₂O (20 mL). After 30 min, the mixture was allowed to reach r.t. and transferred to flask containing a 0° C. cooled a solution of diethyl oxalate (1.185 g, 8.114 mmol) in Et₂O (30 mL). The reaction mixture was stirred at r.t. overnight. It was poured into H₂O (100 mL) and taken up to pH=2 with HCl (10% aqueous solution). The product was extracted with EtOAc (100 mL), and the organic layer was dried over Na₂SO₄ (anhydrous), filtered and concentrated. The crude residue was flash chromatographed on SiO₂ (5→10% EtOAc/hexanes) to furnish 1.6 g of ethyl 2-oxo-5-phenylpentanoate (colourless oil, yield: 89%).

¹H NMR (CDCl₃, 250 MHz) δ ppm: 7.14 (m, 2H), 7.06 (m, 3H), 4.41-4.24 (m, 2H), 2.85 (t, J=7.4 Hz, 2H), 2.66 (t, J=7.4 Hz, 2H), 1.97 (t, J=7.1 Hz, 2H), 1.36 (t, J=7.1 Hz, 3H).

### b) Ethyl 2-hydroxy-5-phenylpentanoate

NaBH₄ (300 mg, 7.932 mmol) was added to 0° C. cooled a solution of ethyl 2-oxo-5-phenylpentanoate (1.52 g, 6.90 mmol) in EtOH (40 mL). After 5 min, the reaction mixture was poured into H₂O (150 mL) and taken up to pH=1 with HCl (10% aqueous solution). The product was extracted with CH₂Cl₂ (2×120 mL), and the organic layer was dried over Na₂SO₄ (anhydrous), filtered and concentrated. The crude residue was flash chromatographed on SiO₂ (5→20% EtOAc/hexanes) to furnish 843 mg of ethyl 2-hydroxy-5-phenylpentanoate (colourless oil, yield: 55%).

¹H NMR (CDCl₃, 250 MHz) δ ppm: 7.21 (m, 2H), 7.12 (m, 3H), 4.31-4.14 (m, 3H), 2.80-2.56 (m, 3H), 1.92-1.59 (m, 4H), 1.29 (t, J=7.1 Hz, 3H).

### c) Ethyl 2-(benzyloxy)-5-phenylpentanoate

BnBr (1.1 mL) was added to a suspension of ethyl 2-hydroxy-5-phenyl pentanoate (830 mg, 3.733 mmol) and Ag₂O (1.20 g, 5.178 mmol) in DMF (30 mL). The reaction mixture was warmed up to 70° C. and stirred at that temperature for 7 h. It was allowed to reach r.t., filtered through Celite (washing with EtOAc) and washed with H₂O (100 mL). The organic layer was dried over Na₂SO₄ (anhydrous), filtered and concentrated. The crude residue was flash chromatographed on SiO₂ (5→15% EtOAc/hexanes) to furnish 680 mg of ethyl 2-(benzyloxy)-5-phenylpentanoate (yellow coloured oil, yield: 58%).

¹H NMR (CDCl₃, 250 MHz) δ ppm: 7.38 (m, 10H), 5.20 (m, 2H), 4.71 (d, J=11.5 Hz, 1H), 4.59-4.08 (m, 2H), 2.59 (m, 2H), 1.84-1.6 (m, 4H), 1.28 (m, 3H).

### d) 2-(Benzyloxy)-5-phenylpentanoic acid

The compound was prepared from ethyl 2-(benzyloxy)-5-phenylpentanoate following the general procedure B, to furnish the compound as a yellow coloured oil after flash chromatography purification (yield: 10%).

¹H NMR (CDCl₃, 250 MHz) δ ppm: 7.39-7.05 (m, 10H), 4.69 (d, J=11.5 Hz, 1H), 4.35 (d, J=11.5 Hz, 1H), 3.80 (m, 1H), 2.56 (m, 2H), 1.36 (m, 4H).

EI MS: m/z=283 (M−1).

### Example 70

### Sodium 2-(benzylthio)-5-phenylpentanoate

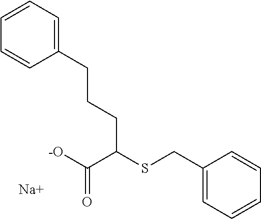

### a) Methyl(benzylthio)acetate

BnBr (3.5 mL, 29.425 mmol) was added to a suspension of K₂CO₃ (5.10 g, 36.90 mmol) and methyl mercaptoacetate (2 mL, 22.366 mmol) in CH₃CN (50 mL). The reaction mixture was stirred at r.t. for 10 min, poured into H₂O (150 mL), and extracted with EtOAc (100 mL). The organic layer was dried over Na₂SO₄ (anhydrous), filtered and concentrated, and the crude residue was flash chromatographed on SiO₂ (0→4% EtOAc/hexanes) to furnish 4.14 g of methyl(benzylthio)acetate (colourless oil, yield: 94%).

### b) Methyl 2-(benzylthio)-5-phenylpentanoate

The compound was prepared from methyl(benzylthio)acetate following the general procedure A, by using PhCH₂CH₂CH₂I as alkylating reagent. Flash chromatography purification afforded the compound as a colourless oil (yield: 77%).

¹H NMR (CDCl₃, 250 MHz) δ ppm: 7.06 (m, 10H), 3.64 (s, 2H), 3.54 (s, 3H), 2.36 (t, J=7.1 Hz, 2H), 1.70 (m, 1H), 1.48 (m, 4H).

### c) 2-(Benzylthio)-5-phenylpentanoic acid

The compound was prepared from methyl 2-(benzylthio)-5-phenylpentanoate following the general procedure B, to furnish the compound as a colourless oil after flash chromatography purification (yield: 11%).

EI MS: m/z=299 (M−1).

### d) Sodium 2-(benzylthio)-5-phenylpentanoate

NaH (16 mg, 60% mineral oil suspension, 0.40 mmol) was added to a solution of 2-(benzylthio)-5-phenylpentanoic acid (123 mg, 0.409 mmol) in THF (10 mL). The reaction mixture was stirred at r.t. for 15 min, and solvent was concentrated off. The crude residue was slurred with Et₂O (5 mL) and hexanes (5 mL), to give 84 mg of sodium 2-(benzylthio)-5-phenylpentanoate (white solid, yield: 64%).

¹H NMR (MeOD, 250 MHz) δ ppm: 7.33 (m, 3H), 7.22 (m, 4H), 7.14 (m, 3H), 3.76 (m, 2H), 3.20 (m, 1H), 2.55 (t, J=7.4 Hz, 2H), 1.86-1.55 (m, 4H).

EI MS: m/z=299 (M−Na−1).

### Example 71

### 2-(Benzylthio)-3-phenylpropanoic acid

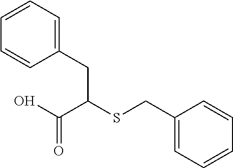

### a) Methyl 2-(benzylthio)-3-phenylpropanoate

The compound was prepared from methyl(benzylthio)acetate following the general procedure A, by using BnBr as alkylating reagent. Flash chromatography purification on SiO₂ (0→10% EtOAc/hexanes) afforded methyl 2-(benzylthio)-3-phenylpropanoate as a colourless oil (yield: 95%).

¹H NMR (CDCl₃, 250 MHz) δ ppm: 7.28 (m, 10H), 3.77 (s, 2H), 3.61 (s, 3H), 3.41 (m, 1H), 3.14 (m, 1H), 2.86 (m, 1H).

### b) 2-(Benzylthio)-3-phenylpropanoic acid

The compound was prepared from methyl 2-(benzylthio)-3-phenylpropanoate following the general procedure B. Flash chromatography purification on SiO₂ (5→20% EtOAc/hexanes), followed by slurries with hexanes, afforded 2-(benzylthio)-3-phenylpropanoic acid as a white solid (yield: 5%).

¹H NMR (CDCl₃, 250 MHz) δ ppm: 7.23 (m, 8H), 7.05 (m, 2H), 3.82 (m, 2H), 3.37 (t, J=8.5 Hz, 1H), 3.16 (m, 1H), 2.85 (m, 1H).

EI MS: m/z=271 (M−1).

### Example 72

### 2-[2-(1,1′-Biphenyl-4-yl)ethyl]-5-phenylpentanoic acid

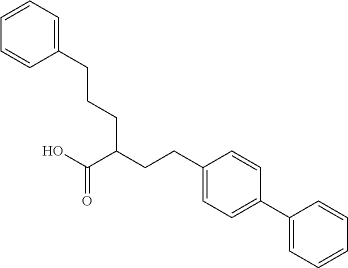

### a) Methyl 2-[2-(1,1′-biphenyl-4-yl)ethyl]-5-phenylpentanoate. (Method F: Suzuki coupling)

Pd(PPh₃)₄ (65 mg, 0.056 mmol) was added to a suspension of methyl 2-{2-(4-{[(trifluoromethyl)sulfonyl]oxy}phenyl)ethyl}-5-phenyl pentanoate (Intermediate A) (210 mg, 0.559 mmol), Cs₂CO₃ (375 mg, 1.150 mmol) and phenylboronic acid (110 mg, 0.902 mmol) in DMF (14 mL). The reaction mixture was stirred at 90° C. until no unreacted Intermediate A was detected by TLC analysis (5 h). It was allowed to reach r.t., poured into H₂O (100 mL) and extracted with Et₂O (100 mL). The organic layer was dried over Na₂SO₄ (anhydrous), filtered and concentrated. The crude residue was purified by flash chromatography on SiO₂ (0→4% EtOAc/hexanes), to afford 54 mg of methyl 2-[2-(1,1′-biphenyl-4-yl)ethyl]-5-phenylpentanoate (colourless oil, yield: 26%).

¹H NMR (CDCl₃, 250 MHz) δ ppm: 7.56 (m, 3H), 7.43 (m, 2H), 7.36-7.12 (m, 9H), 3.71 (s, 3H), 2.60 (m, 4H), 2.45 (m, 1H), 2.00 (m, 1H), 1.88-1.46 (m, 5H).

### b) 2-[2-(1,1′-Biphenyl-4-yl)ethyl]-5-phenylpentanoic acid

The compound was synthesized from methyl 2-[2-(1,1′-biphenyl-4-yl)ethyl]-5-phenylpentanoate following the experimental procedure detailed in Method B. It was purified by flash chromatography on SiO₂ (10→30% EtOAc/hexanes) to furnish a white solid (yield: 44%).

¹H NMR (CDCl₃, 250 MHz) δ ppm: 7.54-7.01 (m, 14H), 2.55 (m, 4H), 2.38 (m, 1H), 1.91 (m, 1H), 1.78-1.38 (m, 5H).

EI MS: m/z=357 (M−1).

### Example 73

### 2-{2-[4′-(2-Furyl)phenyl]ethyl}-5-phenylpentanoic acid

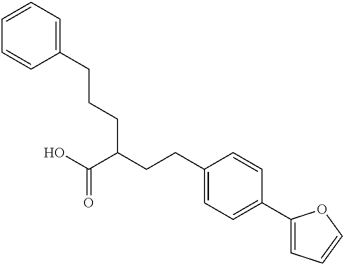

### a) Methyl 2-{2-[4′-(2-furyl)phenyl]ethyl}-5-phenylpentanoate

The compound was synthesized from Intermediate A and furan-2-boronic acid, following the experimental procedure detailed in Method F. It was purified by flash chromatography on SiO₂ (5→10% EtOAc/hexanes) to give a colourless oil (yield: 47%).

¹H NMR (CDCl₃, 250 MHz) δ ppm: 7.69 (d, J=7.9 Hz, 2H), 7.56 (m, 1H), 7.37 (m, 2H), 7.27 (m, 5H), 6.71 (d, J=3.2 Hz, 1H), 6.56 (m, 1H), 3.83-3.76 (m, 3H), 2.76-2.62 (m, 4H), 2.53 (m, 1H), 2.07 (m, 1H), 1.92-1.60 (m, 5H).

### b) 2-{2-[4′-(2-Furyl)phenyl]ethyl}-5-phenylpentanoic acid

The compound was synthesized from methyl 2-{2-[4′-(2-furyl)phenyl]ethyl}-5-phenylpentanoate following the experimental procedure detailed in Method B. It was purified by flash chromatography on SiO₂ (20→100% EtOAc/hexanes) to give a white solid (yield: 75%).

¹H NMR (CDCl₃, 250 MHz) δ ppm: 7.68 (d, J=7.9 Hz, 2H), 7.53 (m, 1H), 7.44-7.20 (m, 7H), 6.69 (d, J=3.2 Hz, 1H), 6.55 (m, 1H), 2.71 (m, 4H), 2.53 (m, 1H), 2.09 (m, 1H), 1.98-1.58 (m, 5H).

EI MS: m/z=347 (M−1).

### Example 74

### 2-{2-[4′-(3-Furyl)phenyl]ethyl}-5-phenylpentanoic acid

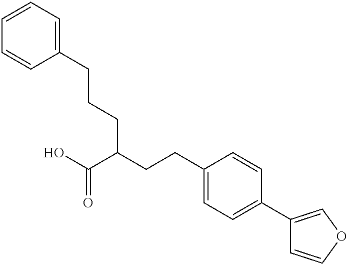

### a) Methyl 2-{2-[4′-(3-furyl)phenyl]ethyl}-5-phenylpentanoate

The compound was synthesized from Intermediate A and furan-3-boronic acid, following the experimental procedure detailed in Method F. It was purified by flash chromatography on SiO₂ (5% EtOAc/hexanes) to give a colourless oil (yield: 27%).

¹H NMR (CDCl₃, 250 MHz) δ ppm: 7.79 (s, 1H), 7.51 (m, 2H), 7.31 (m, 8H), 6.77 (s, 1H), 3.78 (s, 3H), 2.67 (m, 4H), 2.52 (m, 1H), 2.05 (m, 1H), 1.88-1.60 (m, 5H).

### b) 2-{2-[4′-(3-Furyl)phenyl]ethyl}-5-phenylpentanoic acid

The compound was synthesized from methyl 2-{2-[4′-(3-furyl)phenyl]ethyl}-5-phenylpentanoate following the experimental procedure detailed in Method B. It was purified by flash chromatography on SiO₂ (20→100% EtOAc/hexanes) to furnish an off-white solid (yield: 75%).

¹H NMR (CDCl₃, 250 MHz) δ ppm: 7.51 (d, J=8.2 Hz, 2H), 7.39 (d, J=6.8 Hz, 2H), 7.32-7.11 (m, 8H), 2.61 (m, 4H), 2.45 (m, 1H), 1.99 (m, 1H), 1.86-1.50 (m, 5H).

EI MS: m/z=349 (M+1).

### Example 75

### 2-{2-[4′-(3-Thienyl)phenyl]ethyl}-5-phenylpentanoic acid

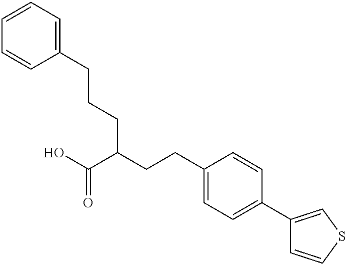

### a) Methyl 2-{2-[4′-(3-thienyl)phenyl]ethyl}-5-phenylpentanoate

The compound was synthesized from Intermediate A and 3-thiopheneboronic acid, following the experimental procedure detailed in Method F. It was purified by flash chromatography on SiO₂ (5→10% EtOAc/hexanes) to afford a colourless oil (yield: 43%).

¹H NMR (CDCl₃, 250 MHz) δ ppm: 7.40 (d, J=8.2 Hz, 2H), 7.31-7.21 (m, 2H), 7.18-6.99 (m, 8H), 3.57 (s, 3H), 2.54-2.41 (m, 4H), 2.32 (m, 1H), 1.83 (m, 1H), 1.64-1.42 (m, 5H).

### b) 2-{2-[4′-(3-Thienyl)phenyl]ethyl}-5-phenylpentanoic acid

The compound was synthesized from methyl 2-{2-[4′-(3-thienyl)phenyl]ethyl}-5-phenylpentanoate following the experimental procedure detailed in Method B. It was purified by flash chromatography on SiO₂ (15→50% EtOAc/hexanes) to give an off-white solid (yield: 80%).

¹H NMR (CDCl₃, 250 MHz) δ ppm: 7.51 (d, J=8.2 Hz, 2H), 7.44-7.35 (m, 2H), 7.31-7.12 (m, 8H), 2.62 (m, 4H), 2.46 (m, 1H), 2.00 (m, 1H), 1.86-1.53 (m, 5H).

EI MS: m/z=363 (M−1).

### Example 76

### 2-{2-[(4′-Pyridinyl)phenyl-4-yl]ethyl}-5-phenylpentanoic acid hydrochloride

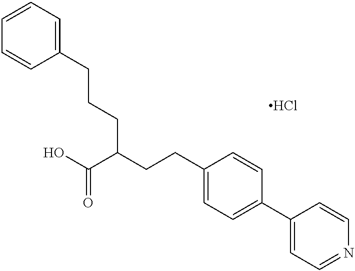

### a) Methyl 2-{2-[(4′-pyridinyl)phenyl-4-yl]ethyl}-5-phenylpentanoate

The compound was synthesized from Intermediate A and 4-pyridineboronic acid, following the experimental procedure detailed in Method F. It was purified by flash chromatography on SiO₂ (20→40% EtOAc/hexanes) to give a yellow-coloured oil (yield: 52%).

¹H NMR (CDCl₃, 250 MHz) δ ppm: 8.64 (m, 2H), 7.52 (m, 5H), 7.28 (m, 4H), 7.16 (m, 2H), 3.70 (s, 3H), 2.59 (m, 4H), 2.45 (m, 1H), 1.97-1.45 (m, 6H).

### b) 2-{2-[(4′-Pyridinyl)phenyl-4-yl]ethyl}-5-phenylpentanoic acid

The compound was synthesized from methyl 2-{2-[(4′-pyridinyl)phenyl-4-yl]ethyl}-5-phenylpentanoate following the experimental procedure detailed in Method B. It was purified by flash chromatography on SiO₂ (0→10% MeOH/CH₂Cl₂) to give a colourless oil (yield: 98%).

¹H NMR (CDCl₃, 250 MHz) δ ppm: 7.99 (bs, 1H), 7.83 (bs, 1H), 7.70 (d, J=7.9 Hz, 1H), 7.54-7.42 (m, 3H), 7.39-7.21 (m, 4H), 7.14 (d, J=7.9 Hz, 2H), 7.08-7.01 (m, 1H), 2.93-2.54 (m, 4H), 2.50 (m, 1H), 2.00 (m, 1H), 1.87-1.62 (m, 5H).

### c) 2-{2-[(4′-Pyridinyl)phenyl-4-yl]ethyl}-5-phenylpentanoic acid hydrochloride

A suspension of 2-{2-[(4′-Pyridinyl)phenyl-4-yl]ethyl}-5-phenylpentanoic acid (0.267 mmol) in HCl.MeOH (0.5 mL, 1 M solution, 0.5 mmol) was stirred at r.t. for 1 h. Solvent was concentrated off, and the crude residue was precipitated from a mixture of MeOH (2 mL) and Et₂O (15 mL), to give 40 mg of a white solid (yield: 38%).

¹H NMR (MeOD, 250 MHz) δ ppm: 8.71 (m, 2H), 8.74 (bs, 2H), 7.83 (m, 2H), 7.36 (bs, 2H), 7.11 (m, 2H), 7.05 (m, 3H), 2.60 (m, 2H), 2.49 (m, 2H), 2.27 (m, 1H), 1.85 (m, 1H), 1.69 (m, 1H), 1.50 (m, 4H).

EI MS: m/z=360 (M−Cl⁻).

### Example 77

### 2-[2-(4-Pyridin-3′-ylphenyl)ethyl]-5-phenylpentanoic acid

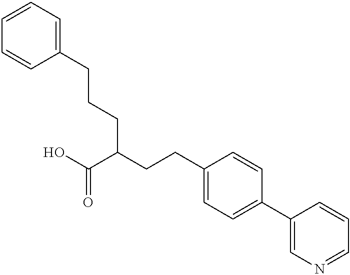

### a) Methyl 2-[2-(4-pyridin-3′-ylphenyl)ethyl]-5-phenylpentanoate

The compound was synthesized from Intermediate A and 3-pyridineboronic acid following the experimental procedure detailed in Method F. It was purified by flash chromatography on SiO₂ (10→40% EtOAc/hexanes) to give a yellow-coloured oil (yield: 49%).

¹H NMR (CDCl₃, 250 MHz) δ ppm: 8.83 (m, 1H), 8.57 (m, 1H), 7.50 (d, J=7.9 Hz, 2H), 7.35 (m, 1H), 7.31-7.23 (m, 4H), 7.16 (m, 3H), 3.69 (s, 3H), 2.60 (m, 4H), 2.45 (m, 1H), 1.99 (m, 1H), 1.84-1.52 (m, 5H).

### b) 2-[2-(4-Pyridin-3′-ylphenyl)ethyl]-5-phenylpentanoic acid

The compound was synthesized from methyl 2-[2-(4-pyridin-3′-ylphenyl)ethyl]-5-phenylpentanoate following the experimental procedure detailed in Method B. It was purified by flash chromatography on SiO₂ (10→60% EtOAc/hexanes) to give a colourless oil (yield: 38%).

¹H NMR (CDCl₃, 250 MHz) δ ppm: 8.86 (bs, 1H), 8.58 (m, 1H), 7.89 (d, J=10.1 Hz, 1H), 7.56-7.07 (m, 10H), 2.65 (m, 4H), 2.47 (m, 1H), 2.03 (m, 1H), 1.90-1.47 (m, 5H).

EI MS: m/z=360 (M+1).

### Example 78

### 5-Phenyl-2-[2-(4′-thien-2-ylphenyl)ethyl]pentanoic acid

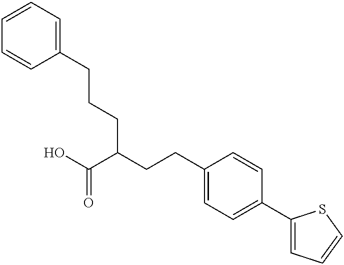

### a) Methyl 2-{2-[4′-(2-thienyl)phenyl]ethyl}-5-phenylpentanoate

The compound was synthesized from Intermediate A and 2-thiopheneboronic acid, following the experimental procedure detailed in Method F. It was purified by flash chromatography on SiO₂ (2→4% EtOAc/hexanes) to give a colourless oil (yield: 56%).

¹H NMR (CDCl₃, 250 MHz) δ ppm: 7.53 (d, J=7.4 Hz, 2H), 7.32-7.22 (m, 4H), 7.21-7.04 (m, 6H), 3.68 (s, 3H), 2.58 (m, 4H), 2.41 (m, 1H), 1.95 (m, 1H), 1.83-1.46 (m, 5H).

### b) 5-Phenyl-2-[2-(4′-thien-2-ylphenyl)ethyl]pentanoic acid

The compound was synthesized from methyl 2-{2-[4′-(2-thienyl)phenyl]ethyl}-5-phenylpentanoate following the experimental procedure detailed in Method B. It was purified by flash chromatography on SiO₂ (10→60% EtOAc/hexanes) to afford a white solid (yield: 38%).

¹H NMR (CDCl₃, 250 MHz) δ ppm: 7.54 (d, J=8.2 Hz, 2H), 7.33-7.13 (m, 9H), 7.06 (m, 1H), 2.63 (m, 4H), 2.45 (m, 1H), 2.00 (m, 1H), 1.86-1.49 (m, 5H).

EI MS: m/z=363 (M−1).

### Example 79

### 5-(1-Naphthyl)-2-[2-(4′-thien-2-ylphenyl)ethyl]-pentanoic acid

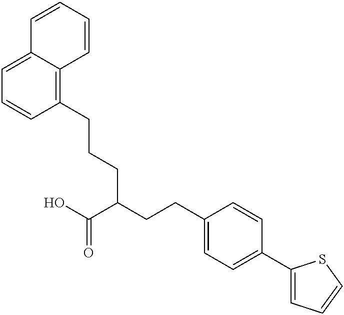

### a) Methyl 5-(1-naphthyl)-2-[2-(4′-thien-2-ylphenyl)ethyl]-pentanoate

The compound was synthesized from Intermediate B and 2-thiopheneboronic acid, following the experimental procedure detailed in Method F. It was purified by flash chromatography on SiO₂ (10→20% EtOAc/hexanes) to afford a yellow-coloured oil (yield: 78%).

¹H NMR (CDCl₃, 250 MHz) δ ppm: 7.99 (m, 1H), 7.83 (m, 1H), 7.70 (d, J=7.9 Hz, 1H), 7.54-7.42 (m, 3H), 7.39-7.21 (m, 5H), 7.14 (d, J=7.9 Hz, 2H), 7.08-7.01 (m, 1H), 3.67 (s, 3H), 3.07 (m, 2H), 2.60 (m, 2H), 2.40 (m, 1H), 1.95 (m, 1H), 1.86-1.62 (m, 5H).

### b) 5-(1-Naphthyl)-2-[2-(4′-thien-2-ylphenyl)ethyl]-pentanoic acid

The compound was synthesized from methyl 5-(1-naphthyl)-2-[2-(4′-thien-2-ylphenyl)ethyl]-pentanoate following the experimental procedure detailed in Method B. It was purified by flash chromatography on SiO₂ (10→30% EtOAc/hexanes) to give a white solid (yield: 20%).

¹H NMR (CDCl₃, 250 MHz) δ ppm: 7.99 (m, 1H), 7.83 (m, 1H), 7.70 (d, J=7.9 Hz, 1H), 7.54-7.42 (m, 3H), 7.39-7.21 (m, 5H), 7.14 (d, J=7.9 Hz, 2H), 7.08-7.01 (m, 1H), 3.07 (m, 2H), 2.63 (m, 2H), 2.47 (m, 1H), 1.95 (m, 1H), 1.87-1.62 (m, 5H).

### Example 80

### 5-(1-Naphthyl)-2-[2-(4′-pyridin-3-ylphenyl)ethyl]-pentanoic acid

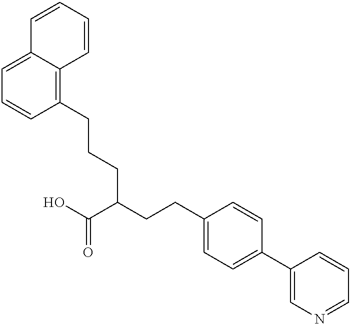

### a) Methyl 5-(1-naphthyl)-2-[2-(4′-pyridin-3-ylphenyl)ethyl]-pentanoate

The compound was synthesized from Intermediate B and 3-pyridineboronic acid, following the experimental procedure detailed in Method F. It was purified by flash chromatography on SiO₂ (20→50% EtOAc/hexanes) to give a colourless oil (yield: 79%).

¹H NMR (CDCl₃, 250 MHz) δ ppm: 8.83 (m, 1H), 8.57 (m, 1H), 7.99 (m, 1H), 7.84 (m, 3H), 7.54-7.23 (m, 9H), 3.68 (s, 3H), 3.07 (m, 2H), 2.80-2.20 (m, 4H), 2.00-1.67 (m, 5H).

### b) 5-(1-Naphthyl)-2-[2-(4′-pyridin-3-ylphenyl)ethyl]-pentanoic acid

The compound was synthesized from methyl 5-(1-naphthyl)-2-[2-(4′-pyridin-3-ylphenyl)ethyl]-pentanoate following the experimental procedure detailed in Method B. It was purified by flash chromatography on SiO₂ (0→30% MeOH/EtOAc) to give a colourless oil (yield: 48%).

¹H NMR (CDCl₃, 250 MHz) δ ppm: 8.66 (s, 1H), 8.38 (d, J=4.9 Hz, 1H), 7.94 (m, 2H), 7.74 (d, J=7.1 Hz, 1H), 7.60 (d, J=7.9 Hz, 1H), 7.48-7.32 (m, 5H), 7.28-7.13 (m, 4H), 2.97 (m, 2H), 2.54 (m, 2H), 2.33 (m, 1H), 1.86 (m, 1H), 1.72-1.56 (m, 5H).

EI MS: m/z=410 (M+1).

### Example 81

### 2-[2-(4-Anilinophenyl)ethyl]-5-phenylpentanoic acid

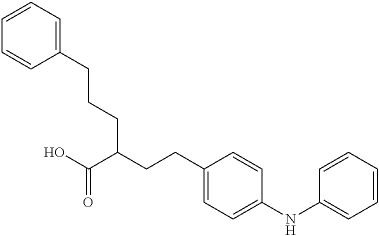

### a) Methyl 2-[2-(4-anilinophenyl)ethyl]-5-phenylpentanoate

Cu(OAc)₂ (150 mg, 0.825 mmol) was added to a solution of methyl 2-[2-(4-aminophenyl)ethyl]-5-phenylpentanoate (100 mg, 0.320 mmol), pyridine (0.13 mL, 1.60 mmol) and phenylboronic acid (58 mg, 0.475 mmol) in CH₂Cl₂ (20 mL). The reaction mixture was stirred at r.t. for 14 h. It was poured into H₂O (150 mL), taken up to pH=6 and extracted with CH₂Cl₂ (2×100 mL). The organic layer was dried over Na₂SO₄ (anhydrous), filtered and concentrated. The crude residue was purified by flash chromatography on SiO₂ (0→8% EtOAc/hexanes), to give 60 mg of methyl 2-[2-(4-anilinophenyl)ethyl]-5-phenylpentanoate (colourless oil, yield: 48%).

¹H NMR (CDCl₃, 250 MHz) δ ppm: 7.36-7.12 (m, 8H), 7.10-6.87 (m, 6H), 5.65 (bs, 1H), 3.70 (s, 3H), 2.67-2.35 (m, 5H), 1.96 (m, 1H), 1.82-1.48 (m, 5H).

### b) 2-[2-(4-Anilinophenyl)ethyl]-5-phenylpentanoic acid

The compound was synthesized from methyl 2-[2-(4-anilinophenyl)ethyl]-5-phenylpentanoate following the experimental procedure detailed in Method B. It was purified by flash chromatography on SiO₂ (20→50% EtOAc/hexanes) to furnish a colourless oil (yield: 17%).

¹H NMR (CDCl₃, 250 MHz) δ ppm: 7.33-7.13 (m, 8H), 7.12-6.85 (m, 6H), 2.61 (m, 4H), 2.44 (m, 1H), 1.93 (m, 1H), 1.88-1.48 (m, 5H).

EI MS: m/z=372 (M−1), 374 (M+1).

### Example 82

### Anti-Proliferative Activity

### Cell Culture

The human colon cancer cell line HCT116 and the human breast cancer cell line MCF-7 were from the American Type Culture Collection (ATCC; CCL-247 and HTB-22, respectively).

Additionally, the following cancer cell lines were used:

| Cell line | ATCC | Culure media |
|---|---|---|
| PC3 | CRL-1435 | DMEM: F12 (Invitrogen) supplemented with 10% fetal |
| (prostate) | | calf serum (Invitrogen), L-glutamine 10M (Invitrogen), |
| | | pyruvate 5M (Invitrogen). |
| MDA-MB-435P | CRL-2914 | DMEM: F12 (Invitrogen) supplemented with 10% fetal |
| (breast) | | calf serum (Invitrogen), L-glutamine 10M (Invitrogen), |
| | | pyruvate 5M (Invitrogen). |
| MDA-MB-468 | HTB-132 | DMEM: F12 (Invitrogen) supplemented with 10% fetal |
| (breast) | | calf serum (Invitrogen), L-glutamine 10M (Invitrogen), |
| | | pyruvate 5M (Invitrogen). |
| Hep3B | HB-8064 | DMEM: F12 (Invitrogen) supplemented with 10% fetal |
| (hepatocarcinoma) | | calf serum (Invitrogen), L-glutamine 10M (Invitrogen), |
| | | pyruvate 5M (Invitrogen). |
| HepG2 | HB-8065 | DMEM: F12 (Invitrogen) supplemented with 10% fetal |
| (hepatocarcinoma) | | calf serum (Invitrogen), L-glutamine 10M (Invitrogen), |
| | | pyruvate 5M (Invitrogen). |
| A549 | CCL-185 | DMEM: F12 (Invitrogen) supplemented with 10% fetal |
| (lung) | | calf serum (Invitrogen), L-glutamine 10M (Invitrogen), |
| | | pyruvate 5M (Invitrogen). |
| K562 | CCL-243 | RPMI (Lonza) supplemented with 10% fetal calf |
| (lymphoma) | | (Invitrogen). |
| SK-mel-98 | HTB-72 | DMEM: F12 (Invitrogen) supplemented with 10% fetal |
| (melanoma) | | calf serum (Invitrogen), L-glutamine 10M (Invitrogen), |
| | | Pyruvat 5M (Invitrogen). |
| U87 | HTB-14 | DMEM: F12 (Invitrogen) supplemented with 10% fetal |
| (glioblastoma) | | calf serum (Invitrogen), L-glutamine 10M (Invitrogen), |
| | | pyruvate 5M (Invitrogen). |
| T98G | CRL-1690 | DMEM: F12 (Invitrogen) supplemented with 10% fetal |
| (glioblastoma) | | calf (Invitrogen), L-glutamine 10M (Invitrogen) |

The NP-9 cell line from pancreas, was provided by Dr. R. Alemany from Translational Research Laboratory (Institut Catala d'Oncologia). (cf., Cascallò, M. et al., “Ras-dependent Oncolysis with an Adenovirus VAI mutant”, Cancer Research, 2003, 63, 5544-5550). This cells was maintained in DMEM:F12 (Invitrogen) supplemented with 10% fetal calf serum (Invitrogen), L-glutamine 10M (Invitrogen), pyruvate 5M (Invitrogen).

The HCT116 cell line was maintained in DMEM GlutaMAX (Invitrogen) supplemented with 10% fetal calf serum and the MCF7 cell line was maintained in RPMI1640 GlutaMAX (Invitrogen) containing 1 mM sodium piruvate and 10% fetal calf serum.

Cells were grown in a humidified incubator at 37° C. in 5% CO₂.

### AlamarBlue Assay

Cells were plated in 96-well plates at a density of 6000 cells/well in 100 μl medium 24 h before addition of drugs. They were then added in concentrations from 100 μM to 0.45 nM (each concentration in triplicate). To do so, a drugs-dilution plate at twice the screening concentrations was prepared. 72 hours later, alamarBlue (Biosource, Invitrogen) viability assay was performed following manufacturer's protocol. In brief, alamarBlue diluted in media was added to cells to have a 5% solution. Cells were incubated at 37° C., 3 hours and at room temperature, 30 min. Cells with no drug and, cells with no drug and lysed with triton X-100 were used as controls. Fluorescence was monitored at 530 nm excitation and 590 nm emission wavelengths. Results were quantified using Infinite F200 Microplate Reader (Tecan Group, Ltd.). EC50 were calculated as the dose of drugs required to inhibit cell growth by 50%, with Origin 7.0 computer program.

The EC50 values (μM) obtained for the compounds of the present invention on HCT-116 and MCF-7 cell lines are summarized in Table 1.

TABLE 1

| | EC₅₀ HCT-116 | | EC₅₀ MCF-7 |
|---|---|---|---|
| Compound | (colon) | Compound | (breast) |
| Example 7 | 680 | Example 7 | 1850 |
| Example 11 | 177 | Example 11 | 268 |
| Example 26 | 1700 | Example 13 | 1800 |
| Example 13 | 2400 | Example 3 | >5000 |
| Example 41 | 644 | Example 5 | 1420 |
| Example 3 | 4510 | Example 9 | 1060 |
| Example 5 | 2420 | Example 12 | 5000 |
| Example 9 | 636 | Example 2 | 696 |
| Example 12 | 5000 | Example 4 | 354 |
| Example 2 | 238 | Example 8 | 5000 |
| Example 4 | 105 | | |
| Example 15 | 85 | | |
| Example 16 | 267 | | |
| Example 70 | 825 | | |
| Example 69 | 965 | | |
| Example 55 | 23 | | |
| Example 28 | 312 | | |
| Example 30 | 58.2 | | |
| Example 35 | 1300 | | |
| Example 19 | 65 | | |
| Example 42 | 56 | | |
| Example 21 | 204 | | |
| Example 39 | 1200 | | |
| Example 36 | 98 | | |
| Example 43 | 75 | | |
| Example 27 | 140.3 | | |
| Example 18 | 95 | | |
| Example 17 | 62 | | |
| Example 57 | 242.2 | | |
| Example 40 | 44.8 | | |
| Example 24 | 75 | | |
| Example 22 | 96.5 | | |
| Example 23 | 67 | | |
| Example 29 | 166 | | |
| Example 56 | 56 | | |
| Example 45 | 165.2 | | |
| Example 46 | 54 | | |
| Example 49 | 140 | | |
| Example 78 | 87.2 | | |
| Example 76 | 166 | | |
| Example 77 | 90 | | |
| Example 52 | 107.2 | | |
| Example 79 | 36.7 | | |
| Example 50 | 103 | | |
| Example 80 | 106.8 | | |
| Example 72 | 88.3 | | |
| Example 73 | 48.6 | | |
| Example 74 | 94.5 | | |
| Example 75 | 184.3 | | |
| Example 38 | 121 | | |
| Example 14 | 256.4 | | |
| Example 44 | 95 | | |
| Example 58 | 159.5 | | |
| Example 48 | 112 | | |
| Example 47 | 23 | | |
| Example 25 | 121 | | |
| Example 33 | 71.4 | | |
| Example 61 | 70.7 | | |
| Example 62 | 155 | | |
| Example 63 | 88 | | |

(All values in μM)

Carboxylate-based compounds have a great solubility which allows administration at the doses required for achiving an effect in-vivo). Carboxylates such as valproic acid showed similar activities in cell-based assays but are progressing in clinical phases for cancer treatments. Moreover, example 12 showed clear inhibition of the target of these compounds (i.e. HDACs).

Panel of cell lines (EC50's) (Table 2)

TABLE 2

| | Example | Example | Example | Example | Example | Example | Example |
|---|---|---|---|---|---|---|---|
| Cell line | 36 | 55 | 11 | 46 | 9 | 19 | 43 |
| PC3 | 168 | 102 | | 55.6 | 364 | | 336 |
| (prostate) | | | | | | | |
| MDA-MB-435P | 125 | 16 | 253 | 52 | 242 | 6.7 | 210 |
| (breast) | | | | | | | |
| MDA-MB-468 | 55.4 | 43 | | 99.8 | 364 | 13 | 87 |
| (breast) | | | | | | | |
| NP-9 | 41 | | | 101.7 | | | |
| (pancreas) | | | | | | | |
| Hep3B | 99.7 | 418.5 | | 107.2 | | | |
| (hepatocarcinoma) | | | | | | | |
| HepG2 | 140 | | | 103 | | | |
| (hepatocarcinoma) | | | | | | | |
| A549 | 104 | | | 62.3 | | | |
| (lung) | | | | | | | |
| K562 | 157 | | | | 472 | | 2.55 |
| (lymphoma) | | | | | | | |
| SK-mel-98 | 170 | | | 78.3 | | | |
| (melanoma) | | | | | | | |
| U87 | 105 | | | 43 | | | |
| (glioblastoma) | | | | | | | |
| T98G | 117.7 | | | 118 | | | |
| (glioblastoma) | | | | | | | |

(All values in μM)
Blank spaces = non tested

### Example 83

### HDAC Inhibition Assay

HDAC inhibition was determined indirectly by measuring the fluorescence generated by deacetylated fluorogenic substrate (KI-104 fluor de Lys™ Biomol®, used at 125 μM) product reacting with a developer solution (KI-105 Fluor de Lys™ Biomol®). All assays were carried out in the assay buffer: 50 mM Tris/Cl, pH 8.0, 137 mM NaCl, 2.7 mM KCl, 1 mM MgCl₂).

Reactions were carried out in a 96-wells microplate (Corning 96 well Flat Bottom Non-binding surface (black) ref. 3651).

Potential inhibitors were added after five fold serial dilutions in DMSO. Final DMSO concentration in the assay microplate was kept at 2%.

Afterwards, human recombinant HDAC1, HDAC 2 or HDAC 8 were added up to 125 nM, 33.3 nM, 25 nM and 1850 nM respectively in assay buffer. When necessary, the mixture was incubated at room temperature prior the addition of substrate.

Finally, substrate was added at 125 nM. Total reaction volume of 50 μl. Human recombinant HDACs could be acquired from commercial sources (HDAC1: ref. #50001, BPS Bioscience™; HDAC 2: ref. #50002, BPS Bioscience™; HDAC 8: ref. #50008, BPS Bioscience™).

Inhibitor-protein incubation, reaction time and reaction temperature are reported in the following Table 3:

TABLE 3

| | Inhibitor-protein | | Reaction |
|---|---|---|---|
| Protein | incubation time | Reaction time | temperature |
| HDAC8 | 0 hours | 3 hours | 37° C. |
| HDAC2 | 2 hours | 1 h 30 min | 37° C. |
| HDAC1 | 0 hours | 3 hours | 25° C. |

(Table. 1 Main parameters of activity assay with histone deacetylase protein)

Reactions were stopped with 50 μl Developer (KI-105 Fluor de Lys™, BIOMOL®) with 2 μmol/L trichostatin A (TSA, final concentration 1 μmol/L). After 20 minutes at 37° C., fluorescence (excitation 360 nm, emission 460 nm) was measured using an Infinite F200 fluorimeter (Tecan). Background was determined in reactions using substrate in the absence of enzyme.

IC₅₀ values are defined as the compound concentration at which the deacetylase activity is 50% inhibited. In these assays the following grading was used (I: IC₅₀≦10 μM; II: 10 μM<IC₅₀≦100 μM; III: 100 μM<IC₅₀≦1 mM) The results obtained are summarized in Table 4.

TABLE 4

| Compound | IC₅₀ on HDAC2 | IC₅₀ on HDAC1 | IC₅₀ on HDAC8 |
|---|---|---|---|
| Example 7 | III | | II |
| Example 11 | III | III | II |
| Example 26 | | | III |
| Example 13 | | | III |
| Example 41 | III | III | II |
| Example 3 | | | III |
| Example 9 | | | III |
| Example 12 | | | III |
| Example 70 | III | | |
| Example 69 | III | | II |
| Example 31 | III | | II |
| Example 55 | II | | I |
| Example 28 | | | III |
| Example 30 | | | III |
| Example 35 | III | | II |
| Example 21 | III | | II |
| Example 39 | | | III |
| Example 36 | II | | II |
| Example 43 | III | | II |
| Example 27 | II | | II |
| Example 57 | III | | II |
| Example 40 | II | | II |
| Example 24 | II | | I |
| Example 22 | III | | II |
| Example 23 | II | | II |
| Example 29 | III | | II |
| Example 45 | II | | I |
| Example 46 | II | | I |
| Example 49 | III | | I |
| Example 53 | II | | |
| Example 68 | III | | |
| Example 78 | II | | |
| Example 76 | II | | |
| Example 77 | II | | I |
| Example 52 | III | | |
| Example 79 | III | | |
| Example 50 | III | | |
| Example 80 | II | | |
| Example 72 | II | | |
| Example 73 | III | | |
| Example 74 | II | | |
| Example 75 | III | | |
| Example 38 | III | | II |
| Example 14 | II | | |
| Example 44 | II | | |
| Example 58 | II | | |
| Example 59 | II | | |
| Example 60 | III | | |
| Example 54 | III | | |
| Example 48 | II | | |
| Example 25 | II | | |
| Example 33 | II | | |
| Example 32 | III | | |
| Example 61 | II | | |
| Example 62 | II | | |
| Example 63 | II | | |
| Example 64 | III | | |
| Example 65 | III | | |
| Example 66 | III | | |
| Example 67 | II | | |

(All values in μM).

Compounds where R₃ is —OR₄ or —OCR₄R₄—O—C(O)—R₄ instead of —OH do not show inhibition of HDACs in inhibition assays because the —OH is required for the binding with the protein. In in-vivo or cell-based conditions the —OR₄ and —OCR₄R₄—O—C(O)—R₄ groups are biotransformed to —OH, thus these groups acts as prodrugs of the carboxylate form.

1. A compound of general formula (I), or a pharmaceutical acceptable salt thereof, or a solvate thereof including a hydrate, or any stereoisomer or mixture of stereoisomers: 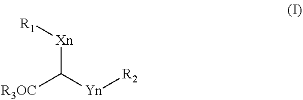
wherein:
R₁ is a radical derived from one of the known ring systems selected from the group consisting of:
one aromatic ring having 5-6 carbon atoms, being optionally one of said carbon atoms replaced by one N, O, or S atom; and
a two fused ring system, wherein
one of the rings is aromatic and the other is aromatic or partially insaturated,
each ring has 5-6 carbon atoms, being optionally 1-3 of said carbon atoms replaced by N, O, or S;
wherein each ring, forming the known ring system, is optionally substituted by at least one radical selected from the group consisting of: (C₁-C₈)alkyl, —OH, halogen, (C₁-C₈)alkoxy, —CN, (C₁-C₈)fluoralkyl, (C₁-C₈)fluoroalkoxy, —CH₂—R₅, —R₁₀, -Qₙ-OR₅, -Qₙ-NR₄C(O)R₅, -Qₙ-C(O)NR₄R₅, -Qₙ-NH₂, -Qₙ-NR₄R₅, -Qₙ-S—R₅, -Qₙ-S(O₂)—R₅, -Qₙ-NR₄S(O₂)R₅, -Qₙ-S(O₂)NR₄R₅, -Qₙ-NR₄—CO, —NR₄R₅, -Qₙ-NR₄—CO—OR₅, and -Qₙ-O—CO—NR₄R₅; where Qₙ is —(CH₂)ₙ—, being n=0, 1, 2 or 3;
R₂ is a phenyl radical optionally substituted by at least one radical selected from the group consisting of: halogen, —OH, —Pₙ—OR₅, —NR₄C(O)R₆, —C(O)NR₄R₆, —NH₂, —NR₄R₅, —R₁₀, —R₆, —CN, (C₁-C₄)fluoralkyl, (C₁-C₄)fluoroalkoxy, (C₁-C₄)alkoxy, and (C₁-C₄)alkyl; where Pₙ is —(CH₂)ₙ— being n=0 or 1
Xₙ represents a birradical selected from the group consisting of: —(CH₂)₁₋₄—, (C₂-C₄)-alkenyl, (C₂-C₄)alkynyl, —S—(CH₂)₁₋₃—#, and —(CH₂)₁₋₃—O—#; wherein the symbol # indicates the position at which Xₙ is attached to R₁;
Yₙ is a birradical selected from the group consisting of: —(CH₂)₂₋₄—, —S—(CH₂)₁₋₃#, and —O—(CH₂)₁₋₃—#; wherein the symbol # indicates the position at which Yₙ is attached to R₂;
R₃ is a radical selected from the group consisting of: —OR₄, —O—CR₄R₄—O—C(O)—R₄;
R₄ is a radical selected from: hydrogen and (C₁-C₄)alkyl optionally substituted by at least one radical selected from the group consisting of: —NH₂ and —OH;
R₅ is a radical selected from: hydrogen, and -Lₙ-R₇, where
Lₙ is —(CH₂)ₙ— with n=0, 1, 2, 3 or 4; and
R₇ is a known ring systems with 1 ring or 2 fused rings, each one of the rings forming said ring system being partially unsaturated or aromatic, have 5-6 members, each member being independently selected from C, N, O, S, CH, CH₂, and NH; and being each ring forming said ring system optionally substituted by at least one radical selected from the group consisting of: (C₁-C₄)-alkyl, —CN, (C₁-C₄)fluoralkyl, (C₁-C₄)fluoroalkoxy, halogen, (C₁-C₄)alkoxy, —NH₂, —OH, dialkyl(C₁-C₄)amino, and a known aromatic ring of 5-6 members independently selected from N, O, S, CH, and NH which is linked to R₇ via a (C₁-C₄)alkyl birradical;
R₆ is a radical selected from the group consisting of: (C₁-C₄)alkyl optionally substituted by at least one radical selected from the group consisting of: halogen, cyano, amino, and an aromatic known ring having 5-6 members independently selected from N, O, S, CH, and NH;
R₁₀ is an aromatic known ring having 5-6 members independently selected from N, O, S, CH, and NH;
with the proviso that when R₁ is phenyl:
R₂ is a phenyl radical substituted by at least one radical selected from the group consisting of: (C₁-C₄)alkoxy, (C₁-C₄)alkyl, —NHC(O)CH₃, halogen, —O—CH₂—R₈, —OH, —NH₂, —OR₁₁, —R₈, —NHR₁₁, and —NH—CH₂-phenyl;
R₈ is a aromatic known ring having 5-6 members independently selected from N, O, S, CH, and NH;
R₁₁ is a phenyl ring optionally substituted with —F, —CF₃, —OCH₃ and —CN;
R₃ is selected from the group consisting of: —OH, (C₁-C₄)alkoxy and —O—CH₂—O—C(O)—CH₃;
Xₙ is selected from the group consisting of: —(CH₂)₃—; and
Yₙ is selected from the group consisting of: —(CH₂)₂;
and with the proviso that R₁ and R₂ are not simultaneously a phenyl substituted by one —OMe radical.

2. A compound according to claim 1, wherein:
R₁ is a radical derived from one of the known ring systems selected from the group consisting of:
one aromatic ring having 5-6 carbon atoms, being optionally one of said carbon atoms replaced by one N, O or S; and
a two fused ring system, wherein
one of the rings is aromatic and the other is aromatic or partially insaturated,
each ring has 5-6 carbon atoms, being optionally 1-3 carbon atoms replaced by N, O, or S;
being each ring, forming said ring system, optionally substituted by at least one radical selected from the group consisting of: (C₁-C₈)alkyl, —OH, halogen, (C₁-C₈)alkoxy, —CN, —(C₁-C₄)fluoralkyl, —(C₁-C₄)fluoroalkoxy, —CH₂—R₅, —R₁₀, -Qₙ-OR₅, -Qₙ-NR₄C(O)R₅, -Qₙ-C(O)NR₄R₅, -Qₙ-NH₂, -Qₙ-NR₄R₅, -Qₙ-NR₄S(O₂)R₅, -Qₙ-S(O₂)NR₄R₅ and -Qₙ-NR₄—CO—OR₅;
where Qₙ is —(CH₂)ₙ—, being n=0 or 1;
R₄ is a radical selected from: hydrogen and (C₁-C₄)alkyl;
R₅ is a radical selected from: hydrogen, and -Lₙ-R₇ where
Lₙ is —(CH₂)ₙ— with n=0 or 1; and
R₇ is an aromatic ring with 5-6 members, each member being independently selected from N, O, S, CH, and NH; and being optionally substituted by at least one radical selected from the group consisting of: —(C₁-C₄)-alkyl, —CN, —(C₁-C₄)fluoralkyl, —(C₁-C₄)fluoroalkoxy, halogen, —(C₁-C₄)alkoxy, —NH₂, —OH and dialkyl(C₁-C₄)amino.

3. A compound according to claim 2, wherein:
R₁ is a radical derived from naphtyl; phenyl and thiophene, being the phenyl and thiophene radical optionally substituted by at least one radical selected from the group consisting of: (C₁-C₄)alkyl, —OH, halogen, (C₁-C₄)alkoxy, —CN, —CF₃, —CHF₂, —OCF₃, —OCHF₂, —NH₂, —R₁₀, —NR₄C(O)R₅, —C(O)NR₄R₅, —NR₄S(O₂)R₅, and —S(O₂)NR₄R₅;
R₂ is a phenyl radical optionally substituted by at least one radical selected from the group consisting of: halogen; —OH, —OR₅, —NHC(O)R₆, —C(O)NHR₆, —NH₂, —NHR₅, —R₁₀, —R₆, —CN, —CF₃, —CHF₂, —OCF₃, —OCHF₂, (C₁-C₄)alkoxy and (C₁-C₄)alkyl;
Xₙ represents a birradical selected from the group consisting of: —(CH₂)₂₋₄—, (C₂-C₄)-alkenyl, (C₂-C₄)alkynyl, and —S—(CH₂)₁₋₃—#, wherein the symbol # indicates the position at which Xₙ is attached to R₁;
Yₙ is a birradical selected from the group consisting of: —(CH₂)₂₋₃—, —S—(CH₂)₁₋₂#, and —(CH₂)₁₋₂—#; wherein the symbol # indicates the position at which Yₙ is attached to R₂;
R₅ is a radical selected from: hydrogen and -Lₙ-R₇,
where Lₙ is —(CH₂)ₙ— being n=0 or 1; and
R₇ is an aromatic known ring system with 5-6 members, each member being independently selected from N, O, S, CH, and NH; and being optionally substituted by at least one radical selected from the group consisting of: (C₁-C₄)-alkyl, —CN, —CF₃, —CHF₂, —OCF₃, —OCHF₂, halogen, (C₁-C₄)alkoxy, —NH₂, —OH and dialkyl(C₁-C₄)amino.

4. The compound according to claim 3, wherein
R₁ is selected from the group consisting: 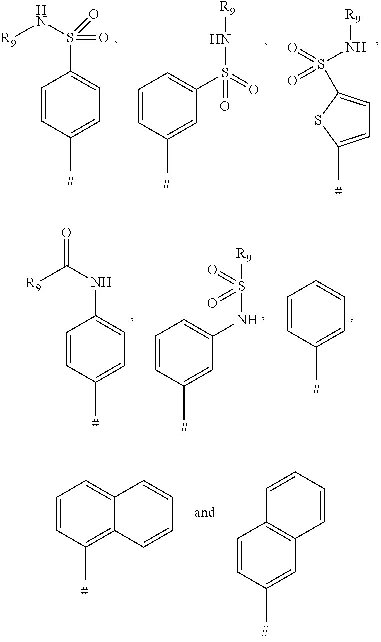
wherein the symbol # indicates the position at which R₁ is attached to Xₙ;
wherein R₉ is —CH₃, —Rₓ or —CH₂—Rₓ;
R₂ is 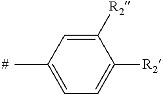
wherein
the symbol # indicates the position at which R₂ is attached to Yₙ;
R″₂ is selected from the group consisting of: hydrogen, —OCH₃, —OH; and
R′₂ is selected from the group consisting of: hydrogen, —NH₂, (C₁-C₄)alkyl, —OH, —OCH₃, —CN, halogen, —NH—CO—CH₃, —O—R₅, —O—CH₂-phenyl, —O—CH₂-pyridine, —NH—CH₂-phenyl, and an aromatic known ring having 5-6 members selected from CH, N, NH, O and S;
R₅ is phenyl optionally substituted by one radical selected from: —CN, —F, —OCH₃, —CF₃;
Rₓ is phenyl optionally substituted by one radical selected from the group consisting of: (C₁-C₄)alkyl, (C₁-C₄)alkoxy, and dialkyl(C₁-C₄)amino;
Xₙ represents a birradical selected from the group consisting of: —(CH₂)₂₋₄—, C₃-alkenyl, —C₃-alkynyl, and —S—(CH₂)₂-#; wherein the symbol # indicates the position at which Xₙ is attached to R₁;
Yₙ is a birradical selected from the group consisting of: —(CH₂)₂₋₃—, —SCH₂—#, and —OCH₂-#; wherein the symbol # indicates the position at which Xₙ is attached to R₂; and
R₃ is hydroxyl, methoxy, ethoxy or —O—CH₂—O—C(O)—CH₃.

5. The compound according to claim 1, wherein
R₁ is selected from the group consisting of: 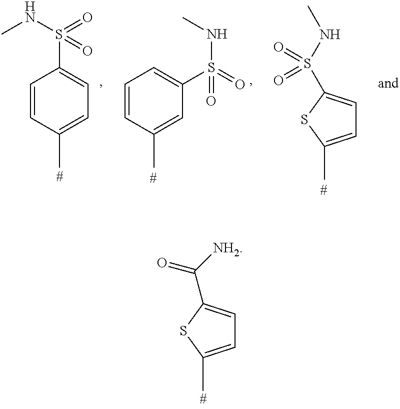
wherein the symbol # indicates the position at which R₁ is attached to Xₙ;
R₂ is selected from the group consisting of: 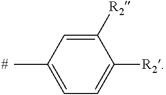
wherein R′₂ and R″₂ are selected from the group consisting of: hydrogen, —NH₂, —OH, —OCH₃, —Br, —CONH₂, and phenyl; and the symbol # indicates the position at which R₂ is attached to Yₙ;
Xₙ is selected from the group consisting of: —(CH₂)—, —(CH₂)₂—, —(CH₂)₃—, wherein the symbol # indicates the position at which Xₙ is attached to R₁;
Yₙ is selected from the group consisting of: —(CH₂)₂— and —S—CH₂—#; wherein the symbol # indicates the position at which Yₙ is attached to R₂; and
R₃ is hydroxyl, methoxy, ethoxy, and —O—CH₂—O—C(O)—CH₃.

6. The compound according to claim 1, wherein
R₁ is phenyl;
R₂ is a phenyl radical substituted by at least one radical selected from the group consisting of: (C₁-C₄)alkoxy, (C₃-C₄)alkyl, —NHC(O)CH₃, halogen, —O—CH₂—R₈, —OH, —NH₂, —OR₁₁, —R₈, —NHR₁₁, and —NH—CH₂-phenyl;
R₈ is a aromatic known ring having 5-6 members independently selected from N, O, S, CH, and NH;
R₁₁ is a phenyl ring optionally substituted with —F, —CF₃, —OCH₃ and —CN;
R₃ is selected from the group consisting of: —OH, (C₁-C₄)alkoxy and —O—CH₂—O—C(O)—CH₃;
Xₙ is selected from the group consisting of: —(CH₂)₃—; and
Yₙ is selected from the group consisting of: —(CH₂)₂.

7. The compound according to claim 1, wherein
R₁ is phenyl substituted by at least one radical selected from —S(O₂)NR₄R₅, —NR₄S(O₂)R₅ and —NR₄C(O)R₅;
R₂ is phenyl optionally substituted by one (C₁-C₄)alkyl radical;
R₃ is selected from the group consisting of: —OH, methoxy, and —O—CH₂—O—C(O)—CH₃;
R₄ is hydrogen; and
R₅ is -Lₙ-R₇, where
Lₙ is —(CH₂)ₙ— being n=0 or 1; and
R₇ is phenyl optionally substituted by at least one radical selected from the group consisting of: (C₁-C₄)-alkyl, (C₁-C₄)alkoxy, and dialkyl(C₁-C₄)amino;
Xₙ is 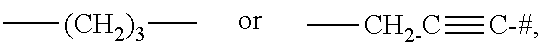
wherein the symbol # indicates the position at which Xₙ is attached to R₁; and
Yₙ is —(CH₂)₂—.

8. The compound according to claim 1, wherein
R₁ is naphtyl;
R₂ is phenyl optionally substituted by one radical selected from the group consisting of: (C₁-C₄)alkyl radical, thiophene and pyridine;
R₃ is —OH;
Xₙ is —(CH₂)₃—; and
Yₙ is —(CH₂)₂—.

9. The compound according to claim 1, wherein
R₁ is thiophene substituted by one —S(O₂)NR₄R₅ radical;
R₂ is phenyl;
R₃ is —OH;
R₄ is hydrogen;
R₅ is a (C₁-C₄)alkyl radical;
Xₙ is —(CH₂)₃—; and
Yₙ is —(CH₂)₂—.

10. The compound according to claim 1, which is selected from the group consisting of:
2-{4-[(Methylamino)sulfonyl]benzyl}-4-phenylbutanoic acid;
5-(4-[(Methylamino)sulfonyl]phenyl)-2-(2-phenylethyl)pentanoic acid;
(Acetyloxy)methyl 5-(3-{[(3,4-dimethoxyphenyl)amino]sulfonyl}phenyl)-2-(2-phenylethyl)pentanoate;
(Acetyloxy)methyl 5-(3-[(4-methylanilinosulfonyl]phenyl)-2-(2-phenylethyl)pentanoate;
(Acetyloxy)methyl 5-(3-[(methylamino)sulfonyl]phenyl)-2-(2-phenylethyl)pentanoate;
Sodium 5-phenyl-2-(2-phenylethyl)pentanoate;
5-(2-Naphthyl)-2-(2-phenylethyl)pentanoic acid;
5-(1-Naphthyl)-2-(2-phenylethyl)pentanoic acid;
5-[3-{[4-(Dimethylamino)benzoyl]amino}phenyl]-2-(2-phenylethyl)pentanoic acid;
5-[3-{[4-(Dimethylamino)benzoyl]amino}phenyl]-2-(2-phenylethyl)pentanoic acid hydrochloride;
5-(3′-{[(4-Methylphenyl)sulfonyl]amino}phenyl)-2-(2-phenylethyl)pentanoic acid;
5-{5-[(Methylamino)sulfonyl]thien-2-yl}-2-(2-phenylethyl)pentanoic acid;
5-(3-[(Benzylamino)sulfonyl]phenyl)-2-(2-phenylethyl)pentanoic acid;
5-Phenyl-2-(2-pyridin-2-ylethyl)pentanoic acid;
2-[2-(3-methoxyphenyl)ethyl]-5-phenylpentanoic acid;
2-[2-(3-Hydroxyphenyl)ethyl]-5-phenylpentanoic acid;
2-{2-[4-(Acetylamino)phenyl]ethyl}-5-phenylpentanoic acid;
2-[2-(4-Aminophenyl)ethyl]-5-phenylpentanoic acid;
2-[2-(4-(Benzylamino)phenyl)ethyl]-5-phenylpentanoic acid;
5-(3-[(4-Methylanilinosulfonyl]phenyl)-2-(2-phenylethyl)pentanoic acid;
Sodium 5-(3-[(4-methylanilinosulfonyl]phenyl)-2-(2-phenylethyl)pentanoate;
5-[3-(4-Methylanilinosulfonyl)phenyl]-2-(2-phenylethyl)pent-4-ynoic acid;
5-(3-[(Anilinosulfonyl]phenyl)-2-(2-phenylethyl)pentanoic acid;
5-(4-[Anilinosulfonyl]phenyl)-2-(2-phenylethyl)pentanoic acid;
5-(3-[(Methylamino)sulfonyl]phenyl)-2-(2-phenylethyl)pentanoic acid;
Methyl 5-(3-[(methylamino)sulfonyl]phenyl)-2-(2-phenylethyl)pentanoate;
5-(3-{[(3,4-dimethoxyphenyl)amino]sulfonyl}phenyl)-2-(2-phenylethyl)pentanoic acid;
5-[3-(4-methylanilinosulfonyl)phenyl]-2-(3-phenylpropyl)pentanoic acid;
2-[2-(4-Ethylphenyl)ethyl]-5-phenylpentanoic acid;
5-[4-(Anilinosulfonyl)phenyl]-2-[2-(4-ethylphenyl)ethyl]pentanoic acid;
Methyl 5-[4-(anilinosulfonyl)phenyl]-2-[2-(4-ethylphenyl)ethyl]pentanoate;
2-[2-(4-Ethylphenyl)ethyl]-6-phenylhexanoic acid;
5-(3-{[4-(Dimethylamino)benzoyl]amino}phenyl)-2-[2-(4-ethylphenyl)ethyl]pentanoic acid;
5-(1-Naphthyl)-2-[2-(4-ethylphenyl)ethyl]-pentanoic acid;
2-[2-(4-hydroxyphenyl)ethyl]-5-phenylpentanoic acid;
2-[2-(4-Benzyloxyphenyl)ethyl]-5-phenylpentanoic acid;
(Acetyloxy)methyl 2-[2-(4-benzyloxyphenyl)ethyl]-5-phenylpentanoate;
2-[2-(4-Methoxyphenyl)ethyl]-5-phenylpentanoic acid;
2-{2-[4-(Pyridin-2-ylmethoxy)phenyl]ethyl}-5-phenylpentanoic acid;
2-{2-[4-(Pyridin-4-ylmethoxy)phenyl]ethyl}-5-phenylpentanoic acid;
2-{2-[4-(2-Cyanophenoxy)phenyl]ethyl}-5-phenylpentanoic acid;
2-{2-[4-(3-cyanophenoxy)phenyl]ethyl}-5-phenylpentanoic acid;
2-{2-[4-(4-cyanophenoxy)phenyl]ethyl}-5-phenylpentanoic acid;
2-{2-[4′-(2-Furyl)phenyl]ethyl}-5-phenylpentanoic acid;
2-{2-[4′-(3-Furyl)phenyl]ethyl}-5-phenylpentanoic acid;
2-{2-[4′-(3-Thienyl)phenyl]ethyl}-5-phenylpentanoic acid;
2-{2-[(4′-Pyridinyl)phenyl-4-yl]ethyl}-5-phenylpentanoic acid;
2-{2-[(4′-Pyridinyl)phenyl-4-yl]ethyl}-5-phenylpentanoic acid hydrochloride;
2-[2-(4-Pyridin-3′-ylphenyl)ethyl]-5-phenylpentanoic acid;
5-Phenyl-2-[2-(4′-thien-2-ylphenyl)ethyl]pentanoic acid;
2-[2-(1,1′-Biphenyl-4-yl)ethyl]-5-phenylpentanoic acid;
5-(1-Naphthyl)-2-[2-(4′-thien-2-ylphenyl)ethyl]-pentanoic acid;
5-(1-Naphthyl)-2-[2-(4′-pyridin-3-ylphenyl)ethyl]-pentanoic acid;
2-{2-[4-(Pyridin-3-ylmethoxy)phenyl]ethyl}-5-phenylpentanoic acid;
2-{2-[4-(4-Fluorophenoxy)phenyl]ethyl}-5-phenylpentanoic acid;
2-{2-[4-Phenoxyphenyl]ethyl}-5-phenylpentanoic acid;
2-{2-[4-(4-Trifluoromethylphenoxy)phenyl]ethyl}-5-phenylpentanoic acid;
2-[2-(4-Bromophenyl)ethyl]-5-phenylpentanoic acid;
2-{2-[4-(4-Methoxyphenoxy)phenyl]ethyl}-5-phenylpentanoic acid;
2-[2-(2,3-Dihydro-1H-indol-3-yl)ethyl]-5-phenylpentanoic acid;
5-(1-Naphthyl)-2-[2-(4′-pyridin-3-ylphenyl)ethyl]-pentanoic acid; and
2-[2-(4-Anilinophenyl)ethyl]-5-phenylpentanoic acid.

11. A pharmaceutical composition comprising:
a therapeutically effective amount of the compound of formula (I) of claim 1, together with appropriate amounts of pharmaceutical excipients or carriers.

12. A medicament comprising a compound selected from the group consisting of:
2-Methyl-4-phenylbutanoic acid;
2-Ethyl-4-phenylbutanoic acid;
2-(2-Phenylethyl)pentanoic acid;
2-Benzyl-4-phenylbutanoic acid;
5-Phenyl-2-(2-phenylethyl)pentanoic acid;
Methyl 2-methyl-4-phenylbutanoate;
Methyl 2-ethyl-4-phenylbutanoate;
(Acetyloxy)methyl 2-benzyl-4-phenylbutanoate;
(Acetyloxy)methyl 5-phenyl-2-(2-phenylethyl)pentanoate;
Sodium 2-(Benzylthio)-5-phenylpentanoate;
2-(Benzyloxy)-5-phenylpentanoic acid;
5-Phenyl-2-propylpentanoic acid;
(4E)-5-Phenyl-2-(2-phenylethyl)pent-4-enoic acid; and
6-Phenyl-2-(2-phenylethyl)hexanoic acid.

13. A method of treating cancer in a subject, wherein the improvement comprises:
utilizing a compound selected from the group consisting of:
2-Methyl-4-phenylbutanoic acid;
2-Ethyl-4-phenylbutanoic acid;
2-(2-Phenylethyl)pentanoic acid;
2-Benzyl-4-phenylbutanoic acid;
5-Phenyl-2-(2-phenylethyl)pentanoic acid;
Methyl 2-methyl-4-phenylbutanoate;
Methyl 2-ethyl-4-phenylbutanoate;
(Acetyloxy)methyl 2-benzyl-4-phenylbutanoate;
(Acetyloxy)methyl 5-phenyl-2-(2-phenylethyl)pentanoate;
Sodium 2-(Benzylthio)-5-phenylpentanoate;
2-(Benzyloxy)-5-phenylpentanoic acid;
5-Phenyl-2-propylpentanoic acid;
(4E)-5-Phenyl-2-(2-phenylethyl)pent-4-enoic acid; and
6-Phenyl-2-(2-phenylethyl)hexanoic acid;
so as to treat the cancer in the subject.

14. A method of treating cancer in a subject, wherein the improvement comprises:
utilizing the compound of claim 1 so as to treat the cancer in the subject.

15. A method of treating cancer in a subject, wherein the improvement comprises:
utilizing the compound of claim 2, to treat the cancer.

16. A method of treating cancer in a subject, wherein the improvement comprises:
utilizing the compound of claim 2 to treat the cancer.

17. A method of treating cancer in a subject, wherein the improvement comprises:
utilizing the compound of claim 3 to treat the cancer.

18. A method of treating cancer in a subject, wherein the improvement comprises:
utilizing the compound of claim 4 to treat the cancer.

19. A method of treating cancer in a subject, wherein the improvement comprises:
utilizing the compound of claim 5 to treat the cancer.

20. A method of treating cancer in a subject, wherein the improvement comprises:
utilizing the compound of claim 6 to treat the cancer.
